SEQUENCE LISTING (204 sequences)

SEQ ID NO: 1 - protein, 10 aa, Homo sapiens
SSYTSSSTLV

SEQ ID NO: 2 - protein, 10 aa, Homo sapiens
SSYTSSSTLG

SEQ ID NO: 3 - protein, 9 aa, Homo sapiens
QSYDSNLRV

SEQ ID NO: 4 - protein, 8 aa, Homo sapiens
QQLNSYPT

SEQ ID NO: 5 - protein, 11 aa, Homo sapiens
NSRDSSGFQLV

SEQ ID NO: 6 - protein, 9 aa, Homo sapiens
QQANSFPIT

SEQ ID NO: 7 - protein, 111 aa, Homo sapiens
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 8 - protein, 6 aa, Homo sapiens
MRAPVI

SEQ ID NO: 9 - protein, 8 aa, Homo sapiens
PWDDVTPP

SEQ ID NO: 10 - protein, 12 aa, Homo sapiens
GFPRITPPSAEI

SEQ ID NO: 11 - protein, 5 aa, Homo sapiens
GFPMP

SEQ ID NO: 12 - protein, 10 aa, Homo sapiens
GFPHSSSVSR

SEQ ID NO: 13 - protein, 11 aa, Homo sapiens
RFPMRHEKTNY

SEQ ID NO: 14 - protein, 8 aa, Homo sapiens
RFPPTATI

SEQ ID NO: 15 - protein, 7 aa, Homo sapiens
TQRRDLG

SEQ ID NO: 16 - protein, 11 aa, Homo sapiens
KFPGGTVRGLK

SEQ ID NO: 17 - protein, 12 aa, Homo sapiens
GFPVIVEERQST

SEQ ID NO: 18 - protein, 10 aa, Homo sapiens
RFPQRVDNRV

SEQ ID NO: 19 - protein, 8 aa, Homo sapiens
TGQSIKRS

SEQ ID NO: 20 - protein, 6 aa, Homo sapiens
LTHPYF

SEQ ID NO: 21 - protein, 6 aa, Homo sapiens
LRPPQS

SEQ ID NO: 22 - protein, 11 aa, Homo sapiens
TSKNTSSSKRH

SEQ ID NO: 23 - protein, 12 aa, Homo sapiens
RYYCRSSDCTVS

SEQ ID NO: 24 - protein, 10 aa, Homo sapiens
FRRMETVPAP

SEQ ID NO: 25 - protein, 277 aa, Homo sapiens
MKYLLPTAAAGLLLLAAQPAMAEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARMRAPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 26 - protein, 464 aa, Homo sapiens
MAWALLLLTLLTQDTGSWADIQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARMRAPVIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTSPVLDSDGSFFLYSKLTVDKSRWQQGNVFSCSVMHEALHNHYTQKSLSLSLGK

SEQ ID NO: 27 - protein, 233 aa, Homo sapiens
MAWALLLLTLLTQDTGSWADAELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 28 - protein, 11 aa, Homo sapiens
FLTYNSYEVPT

SEQ ID NO: 29 - protein, 9 aa, Homo sapiens
TNWYLRPLN

SEQ ID NO: 30 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 31 - protein, 98 aa, Homo sapiens
QVQLVQSGAEVKKPGASVKVSCKASGYIFTDYYMHWVRQAPGQELGWMGRINPNSGGTNYAQKFQGRVTMTRDTSISTAYTELSSLRSEDTATYYCAR

SEQ ID NO: 32 - protein, 98 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 33 - protein, 98 aa, Homo sapiens
QVQLVQSGAEVKKPGASVKVSCKASGYTFTGYYMHWVRQAPGQGLEWMGRINPNSGGTNYAQKFQGRVTSTRDTSISTAYMELSRLRSDDTVVYYCAR

SEQ ID NO: 34 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 35 - protein, 98 aa, Homo sapiens
QVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCAR

SEQ ID NO: 36 - protein, 98 aa, Homo sapiens
  X (1)..(98): Xaa
QVQLVQSGAEVKKLGASVKVSCKASGYTFTGYYMHWVXQAPGQGLEWMGWINPNSGGTNYAQKFQGRVTMTRDTSISTAYMELSRLRSDDTAVYYCAR

SEQ ID NO: 37 - protein, 98 aa, Homo sapiens
QVQLVQSGAEVKKPGASVKVSCKASGYTFTNYCMHWVRQVHAQGLEWMGLVCPSDGSTSYAQKFQARVTITRDTSMSTAYMELSSLRSEDTAMYYCVR

SEQ ID NO: 38 - protein, 98 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 39 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 40 - protein, 98 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAR

SEQ ID NO: 41 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 42 - protein, 98 aa, Homo sapiens
QVQLVQSGAEVKKPGASVKVSCKASGYTFTSYAMHWVRQAPGQRLEWMGWSNAGNGNTKYSQEFQGRVTITRDTSASTAYMELSSLRSEDMAVYYCAR

SEQ ID NO: 43 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 44 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCAR

SEQ ID NO: 45 - protein, 98 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 46 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 47 - protein, 92 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTA

SEQ ID NO: 48 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 49 - protein, 98 aa, Homo sapiens
QVQLVQSGAEVKKPGASVKVSCKASGYTFNSYYMHWVRQAPGQGLEWMGIINPSGGSTSYAQKFQGRVTMTRDTSTSTVYMELSSLRSEDTAVYYCAR

SEQ ID NO: 50 - protein, 98 aa, Homo sapiens
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 51 - protein, 98 aa, Homo sapiens; identical to SEQ ID NO: 50

SEQ ID NO: 52 - protein, 96 aa, Homo sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYY

SEQ ID NO: 53 - protein, 99 aa, Homo sapiens
QITLKESGPTLVKPTQTLTLTCTFSGFSLSTSEWCGWIRQPPGKALEWLALIYWNDDKRYSPSLKSRLTITKDTSKNQVVLTMTNMDPVDTATYYCAHR

SEQ ID NO: 54 - protein, 96 aa, Homo sapiens
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYY

SEQ ID NO: 55 - protein, 96 aa, Homo sapiens
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYY

SEQ ID NO: 56 - protein, 100 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 57 - protein, 100 aa, Homo sapiens
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 58 - protein, 100 aa, Homo sapiens
EVQLLESGGGLVQPGGSLRLSCAASGFTFSDHYMSWVRQAQGKGLELVGLIRNKANSYTTEYAASVKGRLTISREDSKNTLYLQMSSLKTEDLAVYYCAR

SEQ ID NO: 59 - protein, 100 aa, Homo sapiens
EVQLVESGGGLVQPGGSLRLSCAASGFTFSDHYMSWVRQAQGKGLELVGLIRNKANSYTTEYAASVKGRLTISREDSKNTLYLQMSSLKTEDLAVYYCAR

SEQ ID NO: 60 - protein, 98 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAK

SEQ ID NO: 61 - protein, 98 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 62 - protein, 98 aa, Homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAK

SEQ ID NO: 63 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 64 - protein, 100 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 65 - protein, 98 aa, Homo sapiens
EVQLVESGGGLVQPGGSLRLSCPASGFTFSNHYMSWVRQAPGKGLEWVSYISGDSGYTNYADSVKGRFTISRDNANNSPYLQMNSLRAEDTAVYYCVK

SEQ ID NO: 66 - protein, 98 aa, Homo sapiens
EVQLVESGGGLVQPGGSLRLSCAASGFTFSNHYTSWVRQAPGKGLEWVSYSSGNSGYTNYADSVKGRFTISRDNAKNSLYLQMNSLRAEDTAVYYCVK

SEQ ID NO: 67 - protein, 98 aa, Homo sapiens
EVQLVESGGGLVQPGGSLRLSCAASGFTFSNSDMNWVHQAPGKGLEWVSGVSWNGSRTHYADSVKGRFIISRDNSRNTLYLQTNSLRAEDTAVYYCVR

SEQ ID NO: 68 - protein, 97 aa, Homo sapiens
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 69 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 70 - protein, 97 aa, Homo sapiens
EVQLVHSGGGLVQPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 71 - protein, 97 aa, Homo sapiens
EVQLVQSGGGLVQPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 72 - protein, 98 aa, Homo sapiens
EVQLVESGGGLVQPGGSLRLSCSASGFTFSSYAMHWVRQAPGKGLEYVSAISSNGGSTYYADSVKGRFTISRDNSKNTLYVQMSSLRAEDTAVYYCVR

SEQ ID NO: 73 - protein, 35 aa, Homo sapiens
TFSSYAMHWVRQAPGKGLEYVSAISSNGGSTYYAD

SEQ ID NO: 74 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 75 - protein, 98 aa, Homo sapiens; identical to SEQ ID NO: 74

SEQ ID NO: 76 - protein, 98 aa, Homo sapiens; identical to SEQ ID NO: 74

SEQ ID NO: 77 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 78 - protein, 97 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 79 - protein, 98 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCAR

SEQ ID NO: 80 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 81 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 82 - protein, 98 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCAR

SEQ ID NO: 83 - protein, 97 aa, Homo sapiens
EDQLVESGGGLVQPGGSLRPSCAASGFAFSSYVLHWVRRAPGKGPEWVSAIGTGGDTYYADSVMGRFTISRDNAKKSLYLQMNSLIAEDMAVYYCAR

SEQ ID NO: 84 - protein, 98 aa, Homo sapiens
EVQLVESGGGLVQPGGSLRLSCAASGFTFSSYWMHWVRQAPGKGLVWVSRINSDGSSTTYADSVKGRFTISRDNAKNTLYLQMNSLRAEDTAVYYCAR

SEQ ID NO: 85 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 86 - protein, 97 aa, Homo sapiens
QVQLQQWGAGLLKPSETLSLTCAVYGGSFSGYYWSWIRQPPGKGLEWIGEIIHSGSTNYNPSLKSRVTISVDTSKNQFSLKLSSVTAADTAVYYCAR

SEQ ID NO: 87 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 88 - protein, 97 aa, Homo sapiens
QVQLQQWGAGLLKPSETLSLTCAVYGGSVSGYYWSWIRQPPGKGLEWIGYIYYSGSTNNNPSLKSRATISVDTSKNQFSLNLSSVTAADTAVYCCAR

SEQ ID NO: 89 - protein, 99 aa, Homo sapiens
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 90 - protein, 99 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAR

SEQ ID NO: 91 - protein, 99 aa, Homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 92 - protein, 98 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 93 - protein, 98 aa, Homo sapiens
QVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCAR

SEQ ID NO: 94 - protein, 98 aa, Homo sapiens
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 95 - protein, 98 aa, Homo sapiens
QVQLQESGPGLVKPSQTLSLTCAVSGYSISSSNWWGWIRQPPGKGLEWIGYIYYSGSIYYNPSLKSRVTMSVDTSKNQFSLKLSSVTAVDTAVYYCAR

SEQ ID NO: 96 - protein, 98 aa, Homo sapiens
QVQLQESGPGLVKPSETLSLTCVVSGGSISSSNWWSWVRQPPGKGLEWIGEIYHSGNPNYNPSLKSRVTISIDKSKNQFSLKLSSVTAADTAVYYCAR

SEQ ID NO: 97 - protein, 98 aa, Homo sapiens
QVQLQESGPGLVKPSETLSLTCVVSGGSISSSNWWSWVRQPPGKGLEWIGEIYHSGSPNYNPSLKSRVTISVDKSKNQFSLKLSSVTAADTAVYYCAR

SEQ ID NO: 98 - protein, 98 aa, Homo sapiens
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYCCAR

SEQ ID NO: 99 - protein, 98 aa, Homo sapiens
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAR

SEQ ID NO: 100 - protein, 99 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 101 - protein, 99 aa, Homo sapiens
QLQLQESGPGLVKPSETLSLTCTVSGGSISSSSYYWGWIRQPPGKGLEWIGSIYYSGSTYYNPSLKSRVTISVDTSKNHFSLKLSSVTAADTAVYYCAR

SEQ ID NO: 102 - protein, 97 aa, Homo sapiens
QVQLQESGPGLVKPSETLSLTCTVSGGSISSYYWSWIRQPAGKGLEWIGRIYTSGSTNYNPSLKSRVTNSVDTSKNQFSLKLSSVTAADTAVYYCAR

SEQ ID NO: 103 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 104 - protein, 97 aa, Homo sapiens
QVQLQESGPGLVKPSETLSLTCTVSGGSVSSYYWSWIRQPPGKGLEWIGYIYYSGSTNYNPSLKSRVTISVDTSKMQFSLKLSSVTAADTAVYYCAR

SEQ ID NO: 105 - protein, 97 aa, Homo sapiens
QVQLQESGPGLVKPSDTLSLTCTVSGGSISSYYWSWIRQPPGKGLEWIGYIYYSGSTNYNPSLKSRVTISVDTSKNQFSLKLSSVTAADTAVYYCAR

SEQ ID NO: 106 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 107 - protein, 98 aa, Homo sapiens
EVQLVQSGAEVKKPGESLKISCKGSGYSFTSYWIGWVRQMPGKGLEWMGIIYPGDSDTRYSPSFQGQVTISADKPISTAYLQWSSLKASDTAMYYCAR

SEQ ID NO: 108 - protein, 98 aa, Homo sapiens; identical to SEQ ID NO: 106

SEQ ID NO: 109 - protein, 98 aa, Homo sapiens
EVQLLQSAAEVKRPGESLRISCKTSGYSFTSYWIHWVRQMPGKELEWMGSIYPGNSDTRYSPSFQGHVTISADSSSSTAYLQWSSLKASDAAMYYCVR

SEQ ID NO: 110 - protein, 98 aa, Homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 111 - protein, 98 aa, Homo sapiens; identical to SEQ ID NO: 110

SEQ ID NO: 112 - protein, 101 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 113 - protein, 87 aa, Homo sapiens
RKLGASVKVSRKASSYTFTSYDIHCVRQAPGKGLKGWMGGIYSGNGKTGYAQKFQRVTMTRDMSTSTAYMELSSQRSEDIDVYYCAR

SEQ ID NO: 114 - protein, 5 aa, Homo sapiens
DYGMS

SEQ ID NO: 115 - protein, 17 aa, Homo sapiens
GINWNGGSTGYADSVKG

SEQ ID NO: 116 - protein, 11 aa, Homo sapiens
WGQGTLVTVSR

SEQ ID NO: 117 - protein, 11 aa, Homo sapiens
AEDTAVYYCAR

SEQ ID NO: 118 - protein, 11 aa, Homo sapiens
RFTISRDNAKN

SEQ ID NO: 119 - protein, 8 aa, Homo sapiens
GKGLEWVS

SEQ ID NO: 120 - protein, 6 aa, Homo sapiens
WVRQAP

SEQ ID NO: 121 - protein, 11 aa, Homo sapiens
LSCAASGFTFD

SEQ ID NO: 122 - protein, 7 aa, Homo sapiens
AVYYCAR

SEQ ID NO: 123 - protein, 20 aa, Homo sapiens
GGGGSGGGGSGGGGSGGGGS

SEQ ID NO: 124 - protein, 15 aa, Homo sapiens
GGGGSGGGGSGGGGS

SEQ ID NO: 125 - protein, 9 aa, Homo sapiens
NSRDSSGNH

SEQ ID NO: 126 - protein, 8 aa, Homo sapiens
AAWDDSLV

SEQ ID NO: 127 - protein, 8 aa, Homo sapiens
MQSIQLPT

SEQ ID NO: 128 - protein, 9 aa, Homo sapiens
MQSIQLPAT

SEQ ID NO: 129 - protein, 10 aa, Homo sapiens
AAWDDGLSLV

SEQ ID NO: 130 - protein, 10 aa, Homo sapiens
AAWDDSLSGV

SEQ ID NO: 131 - protein, 11 aa, Homo sapiens
NSRDSSGSVRV

SEQ ID NO: 132 - protein, 9 aa, Homo sapiens
LLYYGGAYV

SEQ ID NO: 133 - protein, 11 aa, Homo sapiens
NSRDSSGVSRV

SEQ ID NO: 134 - protein, 10 aa, Homo sapiens
AAWDDSLPYV

SEQ ID NO: 135 - protein, 12 aa, Homo sapiens
AAWDDSLCPEFV

SEQ ID NO: 136 - protein, 11 aa, Homo sapiens
AAWDDSLAWFV

SEQ ID NO: 137 - protein, 10 aa, Homo sapiens
LAWDTSPRWV

SEQ ID NO: 138 - protein, 10 aa, Homo sapiens
TAWDDSLAVV

SEQ ID NO: 139 - protein, 11 aa, Homo sapiens
NSRDSSGNHRV

SEQ ID NO: 140 - protein, 9 aa, Homo sapiens
QQYGSSQRT

SEQ ID NO: 141 - protein, 10 aa, Homo sapiens
AAWDDSLRLV

SEQ ID NO: 142 - protein, 9 aa, Homo sapiens
MQGTHWRPT

SEQ ID NO: 143 - protein, 9 aa, Homo sapiens
MQGKHWPLT

SEQ ID NO: 144 - protein, 9 aa, Homo sapiens
AAWDDSLGF

SEQ ID NO: 145 - protein, 9 aa, Homo sapiens
MQGTHRRAT

SEQ ID NO: 146 - protein, 9 aa, Homo sapiens
MQALQTPLT

SEQ ID NO: 147 - protein, 9 aa, Homo sapiens
MRGTHRRAT

SEQ ID NO: 148 - protein, 9 aa, Homo sapiens
MQGTHWHPT

SEQ ID NO: 149 - protein, 8 aa, Homo sapiens
MQALQSPT

SEQ ID NO: 150 - protein, 10 aa, Homo sapiens
AAWDDSLAFV

SEQ ID NO: 151 - protein, 8 aa, Homo sapiens
MQALQTPT

SEQ ID NO: 152 - protein, 8 aa, Homo sapiens
QQSYSTRT

SEQ ID NO: 153 - protein, 9 aa, Homo sapiens
MQGTHWPFT

SEQ ID NO: 154 - protein, 9 aa, Homo sapiens
MQGTHWPAT

SEQ ID NO: 155 - protein, 10 aa, Homo sapiens
AAWDDSLRSV

SEQ ID NO: 156 - protein, 9 aa, Homo sapiens
AAWDDSLLV

SEQ ID NO: 157 - protein, 11 aa, Homo sapiens
DSWDNSLVSPV

SEQ ID NO: 158 - protein, 9 aa, Homo sapiens
MQALQSPAT

SEQ ID NO: 159 - protein, 9 aa, Homo sapiens
MQALQTPVT

SEQ ID NO: 160 - protein, 11 aa, Homo sapiens
AAWDDSLSAYV

SEQ ID NO: 161 - protein, 11 aa, Homo sapiens
NSRDSSGRVNV

SEQ ID NO: 162 - protein, 8 aa, Homo sapiens
MQALRTRT

SEQ ID NO: 163 - protein, 11 aa, Homo sapiens
AAWDDSLFYPV

SEQ ID NO: 164 - protein, 9 aa, Homo sapiens
MQGTHWPVT

SEQ ID NO: 165 - protein, 8 aa, Homo sapiens
MQGTHWRT

SEQ ID NO: 166 - protein, 10 aa, Homo sapiens
AAWDDSLFYV

SEQ ID NO: 167 - protein, 9 aa, Homo sapiens
MQSIQLPLT

SEQ ID NO: 168 - protein, 11 aa, Homo sapiens
AAWDDSLLGSV

SEQ ID NO: 169 - protein, 9 aa, Homo sapiens
CSYAGSSYV

SEQ ID NO: 170 - protein, 8 aa, Homo sapiens
QQDYNLLT

SEQ ID NO: 171 - protein, 10 aa, Homo sapiens
VLYMGSGSAV

SEQ ID NO: 172 - protein, 9 aa, Homo sapiens
MQRIEFPNT

SEQ ID NO: 173 - protein, 11 aa, Homo sapiens
AAWDDSLACAV

SEQ ID NO: 174 - protein, 8 aa, Homo sapiens
QQANSFRT

SEQ ID NO: 175 - protein, 11 aa, Homo sapiens
AAWDDSLSRPV

SEQ ID NO: 176 - protein, 10 aa, Homo sapiens
AAWDDSLYNV

SEQ ID NO: 177 - protein, 11 aa, Homo sapiens
AAWDDSLNRNV

SEQ ID NO: 178 - protein, 8 aa, Homo sapiens
MQVLQTRT

SEQ ID NO: 179 - protein, 8 aa, Homo sapiens
MQALQTRT

SEQ ID NO: 180 - protein, 8 aa, Homo sapiens
QQSYSTRM

SEQ ID NO: 181 - protein, 8 aa, Homo sapiens
MQALQTLT

SEQ ID NO: 182 - protein, 8 aa, Homo sapiens
MRALQTPT

SEQ ID NO: 183 - protein, 11 aa, Homo sapiens
AAWDDSLPGYV

SEQ ID NO: 184 - protein, 10 aa, Homo sapiens
AAWDDSLGFV

SEQ ID NO: 185 - protein, 10 aa, Homo sapiens
AAWDDSLFLV

SEQ ID NO: 186 - protein, 8 aa, Homo sapiens
MQSIQLRT

SEQ ID NO: 187 - protein, 10 aa, Homo sapiens
AAWDDSLSIV

SEQ ID NO: 188 - protein, 8 aa, Homo sapiens
MQGTHWPT

SEQ ID NO: 189 - protein, 8 aa, Homo sapiens
MQALHTRT

SEQ ID NO: 190 - protein, 9 aa, Homo sapiens
NSRDSSGSV

SEQ ID NO: 191 - protein, 9 aa, Homo sapiens
QQYGSSPYT

SEQ ID NO: 192 - protein, 8 aa, Homo sapiens
QQSYSTRT

SEQ ID NO: 193 - protein, 9 aa, Homo sapiens
QQANSFAAT

SEQ ID NO: 194 - protein, 9 aa, Homo sapiens
QQANSFPAT

SEQ ID NO: 195 - protein, 10 aa, Homo sapiens
VLYMGSGVYV

SEQ ID NO: 196 - protein, 11 aa, Homo sapiens
AAWDDSLWSAV

SEQ ID NO: 197 - protein, 12 aa, Homo sapiens
AAWDDSLPRRLV

SEQ ID NO: 198 - protein, 11 aa, Homo sapiens
AAWDDSLPSGV

SEQ ID NO: 199 - protein, 8 aa, Homo sapiens
MQALQTLT

SEQ ID NO: 200 - protein, 10 aa, Homo sapiens
AAWDDGLLRV

SEQ ID NO: 201 - protein, 10 aa, Homo sapiens
AAWDDSLALV

SEQ ID NO: 202 - protein, 11 aa, Homo sapiens
NSRDSSGFQLV

SEQ ID NO: 203 - protein, 277 aa, Homo sapiens
MKYLLPTAAAGLLLLAAQPAMAEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARLTHPYFWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 204 - protein, 266 aa, Homo sapiens
MKYLLPTAAAGLLLLAAQPAMAEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARMRAPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAKAK

(19) United States
(12) Patent Application Publication
(10) Pub. No.: US 2004/0001839 A1
(43) Pub. Date: Jan. 1, 2004

(54) Multimers - isolated molecules comprising epitopes containing sulfated moieties, antibodies to such epitopes, and uses thereof

(76) Inventors: Avigdor Levanon, Rehovot (IL); Yocheved Hagay, Rehovot (IL); Daniel Plaksin, Rehovot (IL); Tikva Vogel, Rehovot (IL); Abraham Nimrod, Rehovot (IL); Hagit Mar-Haim, Aseret (IL); Ester Szanthon, Rehovot (IL); Tamar Richter, Nes Tziona (IL); Boaz Amit, Kiron (IL); Lena Cooperman, Rishon Lezion (IL); Tuvia Peretz, Hod Hasharon (IL); Janette Lazarovits, Reut (IL)

Correspondence Address: KENYON & KENYON, ONE BROADWAY, NEW YORK, NY, 10004, US

(21) Appl. No.: 10/029,988

(22) Filed: Dec. 31, 2001

Related U.S. Application Data
(60) Provisional application No. 60/258,948, filed on Dec. 29, 2000.

(51) Int. Cl. A61K 39/395; C07K 14/46

(52) U.S. Cl. 424/178.1; 530/391.1

(57) ABSTRACT

The present invention provides epitopes present on cancer cells and important in physiological phenomena such as cell rolling, metastasis, and inflammation. Therapeutic and diagnostic methods and compositions using antibodies capable of binding to the epitopes are provided. Methods and compositions according to the present invention can be used in diagnosis of and therapy for such diseases as cancer, including tumor growth and metastasis, leukemia, auto-immune disease, and inflammatory disease

### FIELD OF THE INVENTION

This application is a Continuation-in-Part application of U.S. provisional application Serial No. 60/258,948, filed on Dec. 29, 2000, the subject matter of which is incorporated by reference hereto.

### FIELD OF THE INVENTION

The present invention relates to epitopes that are present on cells, such as cancer cells, metastatic cells, leukemia cells, and platelets, and that are important in such diverse physiological phenomena as cell rolling, metastasis, inflammation, auto-immune diseases, such as idiopathic thrombocytopenia purpura (ITP), adhesion, thrombosis and/or restenosis, and aggregation. The present invention relates to therapeutic and diagnostic methods and compositions using antibodies directed against such epitopes. The present invention also relates to the field of tissue targeting and identification, with the aid of phage display technology, of peptides and polypeptides that specifically bind to target cells. Such peptides and polypeptides are antibodies and antigen binding fragments thereof, constructs thereof, fragments of either or constructs of a fragment. More particularly, the peptides and polypeptides may have anti-cancer activity, anti-metastatic activity, anti-leukemia activity, anti-viral activity, anti-infection activity, and/or activity against other diseases, such as inflammatory diseases, diseases involving abnormal or pathogenic adhesion, thrombosis and/or restenosis, diseases involving abnormal or pathogenic aggregation, and autoimmune diseases, cardiovascular diseases such as myocardial infarction, retinopathic diseases, diseases caused by sulfated tyrosine-dependent protein-protein interactions, and diseased cells generally.

### BACKGROUND OF THE INVENTION

Antibodies, Phase Display, and Tissue Targeting

Tissue-selective targeting of therapeutic agents is an emerging discipline in the pharmaceutical industry. New cancer treatments based on targeting have been designed to increase the specificity and potency of the treatment, while reducing toxicity, thereby enhancing overall efficacy. Mouse monoclonal antibodies (MAb's) to tumor-associated antigens have been employed—in an attempt to target toxin, radionucleotide, and chemotherapeutic conjugates to tumors. In addition, differentiation antigens, such as CD19, CD20, CD22 and CD25, have been exploited as cancer specific targets in treating hematopoietic malignancies. Although extensively studied, this approach has several limitations. One limitation is the difficulty of isolating appropriate monoclonal antibodies that display selective binding. A second limitation is the need for high antibody immunogenicity as a prerequisite for successful antibody isolation. A third limitation is that the final product comprises non-human sequences, which gives rise to an immune response to the non-human material (e.g., human anti-mouse antibody-HAMA response). The HAMA response often results in a shorter serum half-life and prevents repetitive treatments, thus diminishing the therapeutic value of the antibody. This latter limitation has stimulated interest both in engineering chimeric or humanized monoclonal antibodies of murine origin, and in discovering human antibodies. Another limitation of this approach is that it enables the isolation of only a single antibody species directed against only known and purified antigens. Moreover, this method is not selective insofar as it allows for the isolation of antibodies against cell surface markers that are present on normal, as well as on malignant, cells.

There are many factors that influence the therapeutic efficacy of MAb's for treating cancer. These factors include specificity of antigen expression on tumor cells, level of expression, antigenic heterogeneity and accessibility of the tumor mass. Leukemia and lymphoma have been generally more responsive to treatment with antibodies than solid tumors, such as carcinomas. MAb's rapidly bind to leukemia and lymphoma cells in the bloodstream and easily penetrate to malignant cells in lymphatic tissue, thus making lymphoid tumors excellent candidates for MAb-based therapy. An ideal system entails identifying a MAb that recognizes a marker on the cell surface of stem cells that are producing malignant progeny cells.

Phage libraries are used to select random single chain Fv's (scFv's) that bind to isolated, pre-determined target proteins such as antibodies, hormones and receptors. In addition, the use of antibody display libraries in general, and phage scFv libraries in particular, facilitates an alternative means of discovering unique molecules for targeting specific, yet unrecognized and undetermined, cell surface moieties.

Leukemia, lymphoma, and myeloma are cancers that originate in the bone marrow and lymphatic tissues and are involved in uncontrolled growth of cells. Acute lymphoblastic leukemia (ALL) is a heterogeneous disease that is defined by specific clinical—and immunological characteristics. Like other forms of ALL, the definitive cause of most cases of B-cell ALL (B-ALL) is not known although, in many cases, the disease results from acquired genetic alterations in the DNA of a single cell, causing it to become abnormal and multiply continuously. Prognosis for patients afflicted with B-ALL is significantly worse than for patients with other leukemias, both in children and in adults.

Acute Myelogenous Leukemia (AML) is a heterogeneous group of neoplasms with a progenitor cell that, under normal conditions, gives rise to terminally differentiated cells of the myeloid series (erythrocytes, granulocytes, monocytes, and platelets). As in other forms of neoplasia, AML is associated with acquired genetic alterations that result in replacement of normally differentiated myeloid cells with relatively undifferentiated blasts, exhibiting one or more type of early myeloid differentiation. AML generally evolves in the bone marrow and, to a lesser degree, in the secondary hematopoietic organs. AML primarily affects adults, peaking in incidence between the ages of 15-40 years, but it is also known to affect both children and older adults. Nearly all patients with AML require treatment immediately after diagnosis to achieve clinical remission, in which there is no evidence of abnormal levels of circulating undifferentiated blast cells.

To date, a variety of monoclonal antibodies has been developed that induce cytolytic activity against tumor cells. A humanized version of the monoclonal antibody MuMAb4D5, directed to the extracellular domain of P185—growth factor receptor (HER2)—was approved by the FDA and is being used to treat human breast cancer (U.S. Pat. Nos. 5,821,337 and 5,720,954). Following binding, the antibody is capable of inhibiting tumor cell growth that is dependent on the HER2 growth factor receptor. In addition, a chimeric antibody against CD20, which causes rapid depletion of peripheral B cells, including those associated with lymphoma, was recently approved by the FDA (U.S. Pat. No. 5,843,439). The binding of this antibody to target cells results in complement-dependent lysis. This product has recently been approved and is currently being used in the clinic to treat low-grade B-cell non-Hodgkin's lymphoma.

Several other humanized and chimeric antibodies are under development or are in clinical trials. In addition, a humanized Ig that specifically reacts with CD33 antigen, expressed both on normal myeloid cells as well as on most types of myeloid leukemic cells, was conjugated to the anti-cancer drug calicheamicin, CMA-676 (Sievers et al., Blood Supplement, 308, 504a (1997)). This conjugate, known as the drug Mylotarg®, has recently received FDA approval (Caron et al., Cancer Supplement, 73, 1049-1056 (1994)). In light of its cytolytic activity, an additional anti-CD33 antibody (HumM195), currently in clinical trials, was conjugated to several cytotoxic agents, including the gelonin toxin (McGraw et al., Cancer Immunol. Immunother, 39, 367-374 (1994)) and radioisotopes ¹³¹I (Caron et al., Blood 83, 1760-1768 (1994)), ⁹⁰Y (Jurcic et al., Blood Supplement, 92, 613a (1998)) and ²¹³Bi (Humm et al., Blood Supplement, 38:231P (1997)).

A chimeric antibody against the leukocyte antigen CD45 (cHuLym3) is in clinical studies for treatment of human leukemia and lymphoma (Sun et al., Cancer Immunol. Immunother., 48, 595-602 (2000)). In in vitro assays, specific cell lysis was observed in ADCC (antibody dependent cell-mediated cytotoxicity) assays (Henkart, Immunity, 1, 343-346 (1994); Squier and Cohen, Current Opin. Immunol., 6, 447-452 (1994)).

In contrast to mouse monoclonal humanization and construction of chimeric antibodies, the use of phage display technology enables the isolation of scFv's comprising fully human sequences. A fully human antibody against the human TGFb2 receptor based on a scFv clone derived from phage display technology was recently developed. This scFv, converted into a fully human IgG4 that is capable of competing with the binding of TGFb2 (Thompson et al., J. Immunol Methods, 227, 17-29 (1999)), has strong anti-proliferative activity. This technology, known to one skilled in the art, is more specifically described in the following publications: Smith, Science, 228, 1315 (1985); Scott et al, Science, 249, 386-390 (1990); Cwirla et al., PNAS, 87, 6378-6382 (1990); Devlin et al., Science, 249, 404-406 (1990); Griffiths et al., EMBO J., 13(14), 3245-3260 (1994); Bass et al., Proteins, 8, 309-314 (1990); McCafferty et al., Nature, 348, 552-554(1990); Nissim et al., EMBO J., 13, 692-698 (1994); U.S. Pat. Nos. 5,427,908, 5,432,018, 5,223,409 and 5,403,484, lib.

Ligand for Isolated scFv Antibody Molecules

Platelets, fibrinogen, GPIb, selecting, and PSGL-1 each play an important role in several pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that crossreact with platelets and with these molecules would be useful in the diagnosis and treatment of diseases and disorders involving these and other pathogenic conditions.

Platelets

Platelets are well-characterized components of the blood system and play several important roles in hemostasis, thrombosis and/or restenosis, and restenosis. Damage to blood vessel sets in motion a process known as hemostasis, which is characterized by series of sequential events. The initial reaction to damaged blood vessels is the adhesion of platelets to the affected region on the inner surface of the vessel. The next step is the aggregation of many layers of platelets onto the previously adhered platelets, forming the hemostatic plug. This clump of platelets seals the vessel wall. The hemostatic plug is strengthened by the deposition of fibrin polymers. The clot is degraded only when the damage has been repaired.

Importance of Platelets in Metastasis

Tumor metastasis is perhaps the most important factor limiting the survival of cancer patients. Accumulated data indicate that the ability of tumor cells to interact with host platelets represents one of the indispensable determinants of metastasis. Leslie Oleksowicz, Z. M., “Characterization Of Tumor-Induced Platelet Aggregation: The Role Of Immunorelated GPIb And GPIIb/IIIa Expression By MCF-7 Breast Cancer Cells,” Thrombosis Research 79: 261-274 (1995).

It has been demonstrated that the ability of tumor cells to aggregate platelets correlates with the tumor cells' metastasis potential, and inhibition of tumor-induced platelet aggregation has been shown to correlate with the suppression of metastasis in rodent models. It has been demonstrated that tumor cell interaction with platelets involves membrane adhesion molecules and agonist secretion. Expression of immunorelated platelet glycoproteins has been identified on tumor cell lines. It was demonstrated that platelet immunorelated glycoproteins, GPIb, GPIIb/IIIa. GPIb/IX and the integrin αᵥ subunit are expressed on the surface of breast tumor cell lines. Oleksowicz, Z. M., “Characterization Of Tumor-Induced Platelet Aggregation: The Role Of Immunorelated GPIb And GPIIb/IIIa Expression By MCF-7 Breast Cancer Cells,” Thrombosis Research 79: 261-274 (1995); Kamiyama, M., et al., “Inhibition of platelet GPIIb/IIIa binding to fibrinogen by serum factors: studies of circulating immune complexes and platelet antibodies in patients with hemophilia, immune thrombocytopenic purpura, human immunodeficiency virus-related immune thrombocytopenic purpura, and systemic lupus erythematosus,” J Lab Clin Med 117(3): 209-17 (1991).

Gasic (J. T. B. Gasic et al., Proc. Natl. Acad. Sci. USA 61:46-52 (1968)) and coworkers showed that antibody-induced thrombocitopenia markedly reduced the number and volume of metastases produced by CT26 colon adenocarcinoma, Lewis lung carcinoma, and B16 melanoma. Karpatkin, S., et al., “Role of adhesive proteins in platelet tumor interaction in vitro and metastasis formation in vivo,” J. Clin. Invest. 81(4): 1012-9 (1988); Clezardin, P., et al., “Role of platelet membrane glycoproteins Ib/IX and IIb/IIIa, and of platelet alpha-granule proteins in platelet aggregation induced by human osteosarcoma cells,” Cancer Res. 53(19): 4695-700 (1993). Furthermore, a single polypeptide chain (60 kd) was found to be expressed on surface membrane of HEL cells which is closely related to GPIb and corresponds to an incompletely or abnormally O-glycosylated GPIbα subunit. Kieffer, N., et al., “Expression of platelet glycoprotein Ib alpha in HEL cells,” J. Biol. Chem. 261(34): 15854-62 (1986).

GPIb Complex

Each step in the process of hemostasis requires the presence of receptors on the platelet surface. One receptor that is important in hemostasis is the glycoprotein Ib-IX complex (also known as CD42). This receptor mediates adhesion (initial attachment) of platelets to the blood vessel wall at sites of injury by binding von Willebrand factor (vWF) in the subendothelium. It also has crucial roles in two other platelet functions important in hemostasis: (a) aggregation of platelets induced by high shear in regions of arterial stenosis and (b) platelet activation induced by low concentrations of thrombin.

The GPIb-IX complex is one of the major components of the outer surface of the platelet plasma membrane. The GPIb-IX complex comprises three membrane-spanning polypeptides- a disulfide-linked 130 kDa α-chain and 25 kDa β-chain of GPIb and noncovalently associated GPIX (22 kDa). All four units are presented in equimolar amounts on the platelet membrane, for efficient cell-surface expression and function of CD42 complex, indicating that proper assembly of the three subunits into a complex is required for full expression on the plasma membrane. The α-chain of GPIb consists of three distinct structural domains: (1) a globular N-terminal peptide domain containing leucine-rich repeat sequences and Cys-bonded flanking sequences; (2) a highly glycosylated mucin-like macroglycopeptide domain; and (3) a membrane-associated C-terminal region that contains the disulfide bridge to GPIb β and transmembrane and cytoplasmic sequences.

Several lines of evidence indicate that the vWF and thrombin-binding domain of the GPIb-IX complex reside in a globular region that encompasses approximately 300 amino acids at the amino terminus of GPIbα. The human platelets GPIb-IX complex is a key membrane receptor mediating both platelet function and reactivity. Recognition of subendothelial-bound vWF by GPIb allows platelets to adhere to damaged blood vessels. Further, binding of vWF to GPIbα also induces platelet activation, which may involve the interaction of a cytoplasmic domain of the GPIb-IX with cytoskeleton or phospolipase A2. Moreover, GPIbα contains a high-affinity binding site for α-thrombin, which, by an as-yet poorly defined mechanism, facilitates platelet activation.

The N-terminal globular domain of GPIbα contains a cluster of negatively charged amino. Several lines of evidence indicate that, in transfected CHO cells expressing GPIb-IX complex and in platelet GPIbα, the three tyrosine residues contained in this domain (Tyr-276, Tyr-278, and Tyr-279) undergo sulfation.

Protein Sulfation

Protein sulfation is a widespread posttranslational modification that involves enzymatic covalent attachment of sulfate, either to sugar side chains or to the polypeptide backbone. This modification occurs in the trans-Golgi compartment and, therefore affects only protein that traverses this compartment. Such proteins include secretory proteins, proteins targeted for granules, and the extracellular regions of plasma membrane proteins. Tyrosine is an amino acid residue presently known to undergo sulfation. J. W. Kehoe et al., Chemistry and Biol 7: R57-R61 (2000). Other amino acids, for example threonine, may perhaps also undergo sulfation, particularly in diseased cells.

A number of proteins have been found to be tyrosine-sulfated, but the presence of three or more sulfated tyrosines in a single polypeptide, as was found on GPIb, is not common. GPIbα (CD42), which is expressed by platelets and megakaryocytes mediates platelet attachment to and rolling on subendothelium via binding with vWF, also contains numerous negative charges at its N-terminal domain. Such a highly acidic and hydrophilic environment is thought to be a prerequisite for sulfation because tyrosylprotein sulfotransferase specifically recognizes and sulfates tyrosines adjacent to acidic amino residues. J. R. Bundgaard et al., JBC 272:21700-21705 (1997). Full sulfation of the acidic region of GPIbα yields a region with remarkable density of negative charge—13 negative charges within a 19 amino acid stretch, making it a candidate site for electrostatic interaction with other proteins.

Selectins and PSGL-1

The P-, E-, and L-Selectins are a family of adhesion molecules that, among other functions, mediate rolling of leukocytes on vascular endothelium. P-Selectin is stored in granules in platelets and is transported to the surface after activation by thrombin, histamine, phorbol ester, or other stimulatory molecules. P-Selectin is also expressed on activated endothelial cells. E-Selectin is expressed on endothelial cells, and L-Selectin is expressed on neutrophils, monocytes, T cells, and B cells.

P-Selectin Glycoprotein Ligand-1 (PSGL-1, also called CD162) is a mucin glycoprotein ligand for P-Selectin, E-Selectin, and L-Selectin. PSGL-1 is a disulfide-linked homodimer that has a PACE (Paired Basic Amino Acid Converting Enzymes) cleavage site. PSGL-1 also has three potential tyrosine sulfation sites followed by approximately 15 decamer repeats that are high in proline, serine, and threonine. The extracellular portion of PSGL-1 contains three N-linked glycosylation sites and has numerous sialylated, fucosylated O-linked oligosaccharide branches. K. L. Moore et al., JBC 118:445-456 (1992). Most of the N-glycan sites and many of the O-glycan sites are occupied. The structures of the O-glycans of PSGL-1 from human HL-60 cells have been determined. A subset of these O-glycans are core-2, sialylated and fucosylated structures that are required for binding to selectins. Tyrosine sulfation of an amino-terminal region of PSGL-1 is also required for binding to P-Selectin and L-Selectin. Further, there is an N-terminal propeptide that is probably cleaved post-translationally.

PSGL-1 has 361 residues in HL60 cells, with a 267 residue extracellular region, a 25 residue trans-membrane region, and a 69 residue intracellular region. The sequence encoding PSGL-1 is in a single exon, so alternative splicing should not be possible. However, PSGL-1 in HL60 cells, and in most cell lines, has 15 consecutive repeats of a 10 residue consensus sequences present in the extracellular region, but there are 14 and 16 repeats of this sequence, as well, in polymorphonuclear leukocytes, monocytes, and several other cell lines, including most native leukocytes. PSGL-1 forms a disulfide-bonded homodimer on the cell surface. V. Afshar-Kharghan et al., Blood 97:3306-3312 (2001).

PSGL-1 is expressed on neutrophils as a dimer, with apparent molecular weight of both 250 kDa and 160 kDa, whereas on HL60 the dimeric form is 220 kDa. When analyzed under reducing conditions, each subunit is reduced by half. Differences in molecular mass may be due to polymorphisms in the molecule caused by the presence of different numbers of decamer repeats. Leukocyte Typing VI. Edited by T. Kishimoto et al. (1997).

PSGL-1 is expressed on most blood leukocytes, such as neutrophils, monocytes, leukocytes, subset of B cells, and all T cells and mediates rolling of neutrophils on P-Selectin. Leukocyte Typing VI. Edited by T. Kishimoto et al. (1997). PSGL-1 may also mediate neutrophil-neutrophil interaction via binding with L-Selectin, thereby mediating inflammation. Snapp, et al., Blood 91(1): 154-64 (1998).

PSGL-1 mediates rolling of leukocytes on activated endothelium, on activated platelets, and on other leukocytes and inflammatory sites.

A commercially available monoclonal antibody to human PGSL-1, KPL1, was generated and shown to inhibit the interactions between PGSL-1 and P-selectin and between PGSL-1 and L-selectin. The KPL1 epitope was mapped to the tyrosine sulfation consensus motif of PGSL-1 (YEYLDYD). KPL1 recognizes only this particular epitope and does not cross-react with sulfated epitopes present on other cells, such as B-CLL cells, AML cells, metastatic cells, multiple myeloma cells, and the like.

Leukocyte rolling is important in inflammation, and interaction between P-Selectin (expressed by activated endothelium and on platelets, which may be immobilized at sites of injury) and PSGL-1 is instrumental for tethering and rolling of leukocytes on vessel walls. Ramachandran et al., PNAS 98(18): 10166-71 (2001); Afshar-Kharghan, et al., Blood 97(10): 3306-7 (2001).

Cell rolling is also important in metastasis, and P- and E-Selectin on endothelial cells is believed to bind metastatic cells, thereby facilitating extravasation from the blood stream into the surrounding tissues.

Platelets are also involved in the process of metastasis; when metastatic cancer cells enter the blood stream, multicellular complexes composed of platelets and leukocytes coating the tumor cells are formed. These complexes, which may be referred to as microemboli, aid the tumor cells in evading the immune system. The coating of tumor cells by platelets requires expression of P-selectin by the platelets.

Treatment with heparin, an inhibitor of P- and L-Selectin inhibits tumor cell-platelet interaction. Pretreatment of tumor cells with O-sialoglycoprotease, which removes sialylated, fucosylated mucin ligands, also inhibited tumor cell-platelet complex formation. In vivo experiments indicate that either of these treatments results in greater monocyte association with circulating tumor cells, suggesting that reducing platelet binding increases access by immune cells to circulating tumor cells. Varki and Varki, Braz. J. Biol. Res. 34(6): 711-7 (2001).

PSGL-1 and GPIb share structural similarity, having mucin-like, highly glycosylated ligand binding regions. Afshar-Kharghan, et al., Blood 97(10): 3306-7 (2001).

PSGL-1 has been found on all leukocytes: neutrophils, monocytes, lymphocytes, activated peripheral T-cells, granulocytes, eosinophils, platelets and on some CD34 positive stem cells and certain subsets of B-cells. P-Selectin is selectively expressed on activated platelets and endothelial cells. Interaction between P-Selectin and PSGL-1 promotes rolling of leukocytes on vessel walls, and abnormal accumulation of leukocytes at vascular sites results in various pathological inflammations. Stereo-specific contributions of individual tyrosine sulfates on PSGL-1 are important for the binding of P-Selectin to PSGL-1. Charge is also important for binding: reducing NaCl (from 150 to 50 mM) enhanced binding (Kd ˜75 nM). Tyrosine-sulfation on PSGL-1 enhances, but is not ultimately required for PSGL-1 adhesion on P-Selectin. PSGL-1 tyrosine sulfation supports slower rolling adhesion at all shear rates and supports rolling adhesion at much higher shear rates. (Rodgers S D, et al., Biophys J. 81: 2001-9 (2001)).

Fibrinogen

There are two forms of normal human fibrinogen: fibrinogen γ major and fibrinogen γ prime minor variant, each of which is found in normal individuals. Normal fibrinogen, which is the more abundant form (comprising ˜90% of the fibrinogen found in the body), is composed of two identical 55 kDa alpha (α) chains, two identical 95 kDa beta (β) chains, and two identical 49.5 kDa gamma (γ) chains. Normal variant fibrinogen, which is the less abundant form (comprising 10% of the fibrinogen found in the body), is composed of two identical 55 kDa alpha (α) chains, two identical 95 kDa beta (β) chains, one 49.5 kDa gamma (γ) chain, and one 50.5 kDa gamma prime (γ′) chain. The gamma and gamma prime chains are both coded for by the same gene, with alternative splicing occurring at the 3′ end. Normal gamma chain is composed of amino acids 1-411. Normal variant gamma prime chain is composed of 427 amino acids: amino acids 1-407 are the same as those in the normal gamma chain, and amino acids 408-427 are VRPEHPAETEYDSLYPEDDL. This region is normally occupied with thrombin molecules.

Fibrinogen is converted into fibrin by the action of thrombin in the presence of ionized calcium to produce coagulation of the blood. Fibrin is also a component of thrombi, and acute inflammatory exudates.

Platelets, and molecules (such as fibrinogen, GPIb, selecting, and PSGL-1) that play important roles in cell-cell interactions, cell-matrix interactions, platelet-platelet interactions, platelet-cell interactions, platelet-matrix interactions, cell rolling and adhesion, and hemostasis also play important roles in pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that crossreact with platelets and with these molecules would be useful in the diagnosis and treatment of diseases and disorders involving these and other pathogenic conditions. There is therefore a need to identify common epitopes in or among these molecules and to identify antibodies capable of crossreacting therewith.

Antibodies may be provided in many forms, such as fragments, complexes, and multimers. Examples of antibody fragments include single chain Fv (scFv) fragments and Fab fragments.

It has been established that scFv penetrate tissues and are cleared from the blood more rapidly than a full size antibody because they are smaller in size. Adams, G. P., et al., Br. J. Cancer 77, 1405-1412 (1988); Hudson, P. J., Curr. Opin. Immunol. 11(5), 548-557 (1999); Wu, A. M., et al., Tumor Targeting 4, 47 (1999). Thus, scFv are often employed in diagnostics involving radioactive labels such as tumor imaging to allow for a more rapid clearance of the radioactive label from the body. A number of cancer targeting scFv multimers have recently undergone pre-clinical evaluation for in vivo stability and efficacy. Adams, G. P., et al., Br. J. Cancer 77, 1405-1412 (1988); Wu, A. M., et al., Tumor Targeting 4, 47 (1999).

Single chain Fv (scFv) fragments are comprised of the variable domains of the heavy (V_{H}) and light (V_{L}) chains of an antibody tethered together by a polypeptide linker. The linker is long enough to allow the (V_{H}) and the (V_{L}) domains to fold into a functional Fv domain enabling the scFv to recognize and bind its target with the similar or increased affinity of the parent antibody.

Typically, scFv monomers are designed with the C-terminal end of the V_{H} domain tethered by a polypeptide linker to the N-terminal residue of the V_{L}. Optionally an inverse orientation is employed: the C-terminal end of the V_{L} domain is tethered to the N-terminal residue of V_{H} through a polypeptide linker. Power, B., et al., J. Immun. Meth. 242, 193-204 (2000). The polypeptide linker is typically around fifteen amino acids in length. When the linker is reduced to about three to seven amino acids, the scFvs can not fold into a functional Fv domain and instead associate with a second scFv to form a diabody. Further reducing the length of the linker to less than three amino acids forces the scFv association into trimers or tetramers, depending on the linker length, composition and Fv domain orientations. B. E. Powers, P. J. Hudson, J. Immun. Meth. 242 (2000) 193-194.

Recently, it has been discovered that multivalent antibody fragments such as scFv dimers, trimers, and tetramers often provide higher affinity over the binding of the parent antibody to the target. This higher affinity offers potential advantages including improved pharmaco-kinetics for tumor targeting applications. Additionally, in studying P-Selectin and its ligand PSGL-1, which are involved in tethering and rolling of leukocytes, scientists have concluded that cells expressing dimeric forms of PSGL-1 established more stable rolling adhesions because of this higher binding affinity. These adhesions are more sheer resistant and exhibited less fluctuation in rolling velocities. Ramachandran, et al., PNAS, vol. 98(18): 10166-71 (2001).

The greater binding affinity of these multivalent forms may be beneficial in diagnostics and therapeutic regimens. For example, a scFv may be employed as a blocking agent to bind a target receptor and thus block the binding of the “natural” ligand. In such instances, it is desirable to have a higher affinity association between the scFv and the receptor to decrease chances for disassociation, which may allow an undesirable binding of the natural ligand to the target. In addition, this higher affinity may be useful when the target receptors are involved in adhesion and rolling or when the target receptors are on cells present in areas of high sheer flow, such as platelets.

It is an object of the present invention to provide isolated epitopes that are present on various molecules that are instrumental in processes such as cell rolling, inflammation, immune reactions, infection, autoimmune reactions, metastasis, adhesion, thrombosis and/or restenosis, and aggregation, and which are present on diseased cells, such as AML cells, B-CLL cells, multiple myeloma cells, and metastatic cells.

Another object of the invention is to provide methods of using such isolated epitopes to develop antibodies which recognize and crossreact with epitopes that are present on molecules that are instrumental in processes such as cell rolling, inflammation, immune reactions, infection, autoimmune reactions, metastasis, adhesion, thrombosis and/or restenosis, and aggregation, and which are also present on diseased cells, such as AML cells, B-CLL cells, multiple myeloma cells, and metastatic cells.

Other objectives of the invention include the use of such antibodies in the development and provision of medicaments for the inhibition of cell rolling, inflammation, immune reactions, infection, autoimmune reactions, metastasis, adhesion, thrombosis and/or restenosis, and aggregation, and for the treatment of diseases, such as AML, B-CLL, multiple myeloma, metastasis, cardiovascular diseases such as myocardial infarction, retinopathic diseases, diseases caused by sulfated tyrosine-dependent protein-protein interactions, or other diseases in which such cellular functions or actions play a significant role.

It is an object of this invention to utilize the epitopes and antibodies in methods for diagnosing various disease states of an individual, such as, for example, diseases, such as AML, B-CLL, multiple myeloma, and metastasis or other diseases in which such cellular functions or actions as cell rolling, inflammation, immune reactions, infection, autoimmune reactions, metastasis, adhesion, thrombosis and/or restenosis, and aggregation play a significant role.

It is also an object of the invention to provide multivalent forms of antibodies, fragments, and complexes. More specifically, it is an object of the invention to provide dimers, trimers and tetramers, sometimes referred to herein as diabodies, triabodies, and tetrabodies, respectively.

These and other objectives of the invention are provided herein.

### SUMMARY OF THE INVENTION

The present invention provides epitopes that are found on ligands and receptors that play important roles in such diverse processes as inflammation, immune reactions, metastasis, adhesion, thrombosis, restenosis, and aggregation. Epitopes according to the present invention are also found on leukemia and tumor cells, particularly on leukemias of myeloid origin. Thus, these epitopes are useful targets for the therapeutic mediation of these processes. Antibodies directed against such epitopes are useful as therapeutic agents against cancers (both as anti-tumor agents and as anti-metastatic agents), leukemias, autoimmune diseases, inflammatory diseases, cardiovascular diseases such as myocardial infarction, retinopathic diseases and other diseases mediated by abnormal platelet function, and diseases caused by sulfated tyrosine-dependent protein-protein interactions. The present invention provides such antibodies, compositions comprising the antibodies, and therapeutic and diagnostic methods using the antibodies.

The present invention provides an antibody multimer comprising at least a first and a second antigen binding fragment, wherein the at least first or second antigen binding fragment or both is capable of binding or cross-reacting with an epitope comprising the formula

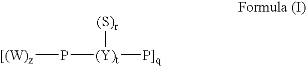

Wherein:

W is any amino acid other than Aspartate and Glutamate

Y is any naturally occurring moiety that is capable of being sulfated

P is (A)ₘ(A)ₙ(X)ᵤ or (X)ᵤ(A)ₙ(A)ₘ or (A)ₙ(X)ᵤ(A)ₘ or (A)ₙ(A)ₘ(X)ᵤ or (X)ᵤ(A)ₘ(A)ₙ or (A)ₘ(X)ᵤ(A)ₙ

S is sulfate or a sulfated molecule

X is any amino acid except Aspartate, Glutamate, or Tyrosine

A is any negatively charged amino acid or leucine, isoleucine, proline, phenylalanine, serine, or glycine

q is 1 to 6

z is 0, 1, or 2

r is 0 or 1

t is 1, 2or 3

u is 0 to 2

n is 0 to 3

m is 0to 3

wherein if n=0 then m>0; wherein if m=0 then n>0; wherein if q is 1, r is 1, and if q is>1 at least one of Y is sulfated.

The present invention also provides an antibody multimer comprising at least a first and second antigen binding fragment, wherein the first or second antigen binding fragment or both is capable of binding or cross-reacting with an epitope comprising the formula

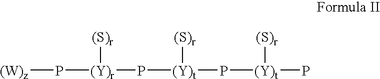

Wherein:

W is any amino acid other than Aspartate and Glutamate

Y is any naturally occurring moiety that is capable of being sulfated

P is (A)ₘ(A)ₙ(X)ᵤ or (X)ᵤ(A)ₙ(A)ₘ or (A)ₙ(X)ᵤ(A)ₘ or (A)ₙ(A)ₘ(X)ᵤ or (X)ᵤ(A)ₘ(A)ₙ or (A)ₘ(X)ᵤ(A)ₙ

S is a sulfate or a sulfated molecule

X is any amino acid except Aspartate, Glutamate or Tyrosine

A is any negatively charged amino acid or leucine, isoleucine, proline, phenylalanine, serine, or glycine

z is 0, 1, or 2

r is 0 or 1

t is 1, 2or 3

u is 0to 2

n is 0to 3

m is 0to 3

wherein if n=0 then m>0; wherein if m=0 then n>0; wherein at least one Y is sulfated.

The present invention also provides an antibody multimer of claim 4 wherein the first or second antigen binding fragment or both binds or cross reacts with the epitope in which: W is Glycine; Y is a peptide conjugate of Tyrosine or a glyco conjugate of Asparagine, Serine or Threonine; A is Glutamate, y Carboxy Glutamate or Aspartate, Leucine, Isoleucine, Proline, Phenylalanine, Serine, or Glycine.

Also provided by the present invention is an antibody multimer comprising at least a first and second antigen binding fragment, wherein the at least first or second antigen binding fragment or both is capable of binding or cross-reacting with an epitope comprising the formula

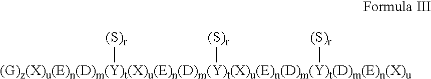

Wherein:

G is Glycine

E is Glutamate

D is Aspartate

Y is Tyrosine

S is sulfate or a sulfated molecule

X is any amino acid except the above

z is 0, 1, or 2

t is 1, 2or 3

r is 0 or 1

u is 0 to 2

n is 0 to 3

m is 0 to 3

wherein at least one Y is sulfated; wherein if n=0 then m>0; wherein if m=0 then n>0.

In certain embodiments of the invention the antibody multimers may be dimers, trimers or tetramers. Dimers of the invention include dimers wherein at least one of the first and second antigen binding fragments is selected from scFv fragments of Y1 and Y17. The antibody fragments of the dimers may be linked by a disulfide bridge, a polypeptide linker and in one embodiment, the polypeptide linker is Gly₄Ser.

In another embodiment of the invention the antibody multimer is a trimer. Trimers of the invention comprise three antigen binding fragments, wherein at least one antigen binding fragment is a Y1 or Y17 scFv fragment. In one embodiment the antigen binding fragments of the trimers of the invention are linked by a polypeptide linker, preferably having 1 to 5 amino acids in the linker.

In another embodiment of the invention the antibody multimer is a tetramer. Tetramers of the invention comprise four antigen binding fragments, and in one embodiment at least one of the antigen binding fragments is a Y1 or Y17 scFv fragment. The antigen binding fragments of the tetramers may be linked by a polypeptide, or may form a complex through streptavidin-biotin association.

In certain embodiments of the invention, the at least first or second antigen binding fragment of the antibody multimer comprises a first hypervariable region comprising SEQ ID NO:8; or the at least first or second antigen binding fragment or both comprises a first hypervariable region comprising SEQ ID NO:20; or the at least first or second antigen binding fragment or both has a second hypervariable region comprising SEQ ID NO:115 and/or a third hypervariable region comprising SEQ ID NO:114.

In yet anther embodiment of the invention, the antibody multimer is capable of binding to at least two different molecules selected from the group consisting of PSGL-1, fibrinogen gamma prime (γ′), GPIbα, heparin, lumican, complement compound 4 (CC4), interalpha inhibitor, and prothrombin. In another embodiment the antibody multimer is capable of binding to at least two different molecules selected from the group consisting of PSGL-1, fibrinogen gamma prime (γ′), GPIbα, heparin, lumican, complement compound 4 (CC4), interalpha inhibitor, and prothrombin and is capable of binding to at least one cell type selected from the group consisting of B-CLL cells, AML cells, multiple myeloma cells, and metastatic cells.

The invention also provides an antibody dimer comprising a first and second antigen binding fragment, wherein said first or second antigen binding fragment or both comprise a hypervariable region comprising the amino acid sequence of SEQ ID NO:20 [Y17 CDR3]. Similarly, the invention provides an antibody dimer comprising a first and second antigen binding fragment, wherein said first or second antigen binding fragment or both comprise a hypervariable region comprising the amino acid sequence of SEQ ID NO:8 [Y1 CDR3].

The invention also provides an antibody multimer comprising a first and second antigen binding fragment, wherein said first or second antigen binding fragment or both is capable of cross-reacting with two or more epitopes, each epitope comprising one or more sulfated tyrosine residues and at least one cluster of two or more acidic amino acids.

In another aspect of the invention there is provided a pharmaceutical composition comprising an antibody multimer according to the invention. In one embodiment the pharmaceutical composition comprises an antibody multimer according to the invention coupled or complexed with an agent selected from the group consisting of anti-cancer, anti-metastasis, anti-leukemia, anti-disease, anti-adhesion, anti-thrombosis, anti-restenosis, anti-auto-immune, anti-aggregation, anti-bacterial, anti-viral, and anti-inflammatory agents. Also provide are pharmaceutical compositions wherein the agent is selected from the group consisting of toxins, radioisotopes and pharmaceutical agents.

In a preferred embodiment the pharmaceutical composition of the invention comprises agent selected from an anti-viral agent selected from the group consisting of acyclovir, ganciclovir and zidovudine. In other embodiments the pharmaceutical composition comprises an agent selected from anti-thrombosis/anti-restenosis agents selected from the group consisting of cilostazol, dalteparin sodium, reviparin sodium, and aspirin; or the pharmaceutical composition comprises an anti-inflammatory agent selected from the group consisting of zaltoprofen, pranoprofen, droxicam, acetyl salicylic 17, diclofenac, ibuprofen, dexibuprofen, sulindac, naproxen, anitolmetin, celecoxib, indomethacin, rofecoxib, and nimesulid; or an anti-autoimmune agent selected from the group consisting of leflunomide, denileukin diftitox, subreum, WinRho SDF, defibrotide, and cyclophosphamide; or an anti-adhesion/anti-aggregation agent selected from the group consisiting of limaprost, clorcromene, and hyaluronic acid; or a pharmaceutical agent selected from the group consisting of doxorubicin, methoxymorpholinyldoxorubicin (morpholinodoxorubicin), adriamycin, cis-platinum, taxol, calicheamicin, vincristine, cytarabine (Ara-C), cyclophosphamide, prednisone, daunorubicin, idarubicin, ludarabine, chlorambucil, interferon alpha, hydroxyurea, temozolomide, thalidomide and bleomycin, and derivatives and combinations thereof. In another embodiment of the invention the pharmaceutical agent is coupled to or complexed with a vehicle or carrier that is capable of being coupled or complexed to more than one agent. In a preferred embodiment the the vehicle or carrier is selected from the group consisting of dextan, lipophiolic polymers, HPMA and liposomes.

Also provided by the invention is the use of an antibody multimer according to the claims in the manufacture of a medicament that inhibits cell-rolling; that inhibits inflammation; that inhibits auto-immune disease; that inhibits restenosis; that inhibits thrombosis; that inhibits metastasis; that inhibits growth and/or replication of tumor cells; that increases the mortality rate of tumor cells that inhibits growth and/or replication of leukemia cells; that increases the mortality rate of leukemia cells; that increases the susceptibility of diseased cells to damage by anti-disease agents; or that increases the susceptibility of tumor cells to damage by anti-cancer agents.

Also provided are kits containing at least one of the antibody multimers of the invention.

Definitions:

Antibodies (Ab's), or immunoglobulins (IgG's), are protein molecules that bind to antigen. They are composed of units of four polypeptide chains (2 heavy and 2 light) linked together by disulfide bonds. Each of the chains has a constant and variable region. They can be divided into five classes, IgG, IgM. IgA, IgD, and IgE, based on their heavy chain component. The IgG class encompasses several sub-classes including, but not restricted to, IgG₁, IgG₂, IgG₃, and IgG₄. Immunoglobulins are produced in vivo by B lymphocytes and recognize a particular foreign antigenic determinant and facilitate clearing of that antigen.

Antibodies may be produced and used in many forms, including antibody complexes. As used herein, the term “antibody complex” or “antibody complexes” is used to mean a complex of one or more antibodies with another antibody or with an antibody fragment or fragments, or a complex of two or more antibody fragments. Examples of antibody fragments include Fv, F(ab′)₂, F(ab′), Fc, and Fd fragments.

As used herein in the specification and in the claims, an Fv is defined as a molecule that is made up of a variable region of a heavy chain of a human antibody and a variable region of a light chain of a human antibody, which may be the same or different, and in which the variable region of the heavy chain is connected, linked, fused or covalently attached to, or associated with, the variable region of the light chain. The Fv can be a single chain Fv (scFv) or a disulfide stabilized Fv (dsFv). An scFv is comprised of the variable domains of each of the heavy and light chains of an antibody, linked by a flexible amino-acid polypeptide spacer, or linker. The linker may be branched or unbranched. Preferably, the linker is 0-15 amino acid residues, and most preferably the linker is (Gly₄Ser)₃.

The Fv molecule itself is comprised of a first chain and a second chain, each chain comprising a first, second and third hypervariable region. The hypervariable loops within the variable domains of the light and heavy chains are termed Complementary Determining Regions (CDR). There are CDR1, CDR2 and CDR3 regions in each of the heavy and light chains. These regions are believed to form the antigen binding site and can be specifically modified to yield enhanced binding activity. The most variable of these regions in nature being the CDR3 region of the heavy chain. The CDR3 region is understood to be the most exposed region of the Ig molecule and as shown and provided herein is the site primarily responsible for the selective and/or specific binding characteristics observed.

A fragment of an Fv molecule is defined as any molecule smaller than the original Fv that still retains the selective and/or specific binding characteristics of the original Fv. Examples of such fragments include but are limited to (1) a minibody, which comprises a fragment of the heavy chain only of the Fv, (2) a microbody, which comprises a small fractional unit of antibody heavy chain variable region (PCT Application No. PCT/IL99/00581), (3) similar bodies comprising a fragment of the light chain, and (4) similar bodies comprising a functional unit of a light chain variable region.

As used herein the term “Fab fragment” is a monovalent antigen-binding fragment of an immunoglobulin. A Fab fragment is composed of the light chain and part of the heavy chain.

A F(ab′)₂ fragment is a bivalent antigen binding fragment of an immunoglobulin obtained by pepsin digestion. It contains both light chains and part of both heavy chains.

A Fc fragment is a non-antigen-binding portion of an immunoglobulin. It contains the carboxy-terminal portion of heavy chains and the binding sites for the Fc receptor.

A Fd fragment is the variable region and first constant region of the heavy chain of an immunoglobulin.

Polyclonal antibodies are the product of an immune response and are formed by a number of different B-lymphocytes. Monoclonal antibodies are derived from a single cell.

A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed or attached at one or both ends.

The term “epitope” is used herein to mean the antigenic determinant or antigen site that interacts with an antibody, antibody fragment, antibody complex or a complex comprising a binding fragment thereof or T-cell receptor. The term epitope is used interchangeably herein with the terms ligand, domain, and binding region.

Selectivity is herein defined as the ability of a targeting molecule to choose and bind one cell type or cell state from a mixture of cell types or cell states, all cell types or cell states of which may be specific for the targeting molecule.

The term “affinity” as used herein is a measure of the binding strength (association constant) between a receptor (e.g., one binding site on an antibody) and a ligand (e.g., antigenic determinant). The strength of the sum total of noncovalent interactions between a single antigen-binding site on an antibody and a single epitope is the affinity of the antibody for that epitope. Low affinity antibodies bind antigen weakly and tend to dissociate readily, whereas high-affinity antibodies bind antigen more tightly and remain bound longer. The term “avidity” differs from affinity because the former reflects the valence of the antigen-antibody interaction.

Specificity of antibody-antigen interaction: Although the antigen-antibody reaction is specific, in some cases antibody elicited by one antigen can cross-react with another unrelated antigen. Such cross-reactions occur if two different antigens share an homologous or similar epitope or an anchor region thereof or if antibodies specific for one epitope bind to an unrelated epitope possessing similar chemical properties.

A platelet is a disc-like cytoplasmic fragment of a megakaryocyte that is shed in the marrow sinus and subsequently are circulating in the peripheral blood stream. Platelets have several physiological functions including a major role in clotting. A platelet contains granules in the central part and peripherally, clear protoplasm, but no definite nucleus.

Agglutination as used herein means the process by which suspended bacteria, cells, discs, or other particles of similar size are caused to adhere and form into clumps. The process is similar to precipitation but the particles are larger and are in suspension rather than being in solution.

The term aggregation means a clumping of platelets induced in vitro, and thrombin and collagen, as part of a sequential mechanism leading to the formation of a thrombus or hemostatic plug.

Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polar, non-polar) such that the substitutions do not substantially in a major way alter peptide, polypeptide or protein characteristics (e.g., charge, IEF, affinity, avidity, conformation, solubility) or activity. Typical substitutions that may be performed for such conservative amino acid substitution may be among the groups of amino acids as follows:

(i) glycine (G), alanine (A), valine (V), leucine (L) and isoleucine (I)

(ii) aspartic acid (D) and glutamic acid (E)

(iii) alanine (A), serine (S) and threonine (T)

(iv) histidine (H), lysine (K) and arginine (R)

(v) asparagine (N) and glutamine (Q)

(vi) phenylalanine (F), tyrosine (Y) and tryptophan (W)

Conservative amino acid substitutions can be made in, as well as, flanking the hypervariable regions primarily responsible for the selective and/or specific binding characteristics of the molecule, as well as other parts of the molecule, e.g., variable heavy chain cassette. Additionally or alternatively, modification can be accomplished by reconstructing the molecules to form full-size antibodies, diabodies (dimers), triabodies (timers) and/or tetrabodies (tetramers) or to form minibodies or microbodies.

A phagemid is defined as a phage particle that carries plasmid DNA. Phagemids are plasmid vectors designed to contain an origin of replication from a filamentous phage, such as m13 of fd. Because it carries plasmid DNA, the phagemid particle does not have sufficient space to contain the full complement of the phage genome. The component that is missing from the phage genome is information essential for packaging the phage particle. In order to propagate the phage, therefore, it is necessary to culture the desired phage particles together with a helper phage strain that complements the missing packaging information.

A promoter is a region on DNA at which RNA polymerase binds and initiates transcription.

A phage display library (also termed phage peptide/antibody library, phage library, or peptide/antibody library) comprises a large population of phage (generally 10⁸-10⁹), each phage particle displaying a different peptide or polypeptide sequence. These peptide or polypeptide fragments may constructed to be of variable length. The displayed peptide or polypeptide can be derived from, but need not be limited to, human antibody heavy or light chains.

A pharmaceutical composition refers to a formulation which comprises a peptide or polypeptide of the invention and a pharmaceutically acceptable carrier, excipient or diluent thereof.

A pharmaceutical agent refers to an agent that is useful in the prophylactic treatment or diagnosis of a mammal including, but not restricted to, a human, bovine, equine, porcine, murine, canine, feline, or any other warm-blooded animal. The pharmaceutical agent is selected from the group comprising radioisotope, toxin, oligonucleotide, recombinant protein, antibody fragment, and anti-cancer agent. Examples of such pharmaceutical agents include, but are not limited to anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

An anti-leukemia agent is an agent with anti-leukemia activity. For example, anti-leukemia agents include agents that inhibit or halt the growth of leukemic or immature pre-leukemic cells, agents that kill leukemic or pre-leukemic, agents that increase the susceptibility of leukemic or pre-leukemic cells to other anti-leukemia agents, and agents that inhibit metastasis of leukemic cells. In the present invention, an anti-leukemia agent may also be agent with anti-angiogenic activity that prevents, inhibits, retards or halts vascularization of tumors.

The expression pattern of a gene can be studied by analyzing the amount of gene product produced under various conditions, at specific times, in various tissues, etc. A gene is considered to be “over expressed” when the amount of gene product is higher than that found in a normal control, e.g., non-diseased control.

A given cell may express on its surface a protein having a binding site (or epitope) for a given antibody, but that binding site may be exist in a cryptic form (e.g., be sterically hindered or be blocked, or lack features needed for binding by the antibody) in the cell in a state, which may be called a first stage (stage I). Stage I may be, for example, a normal, healthy, non-diseased status. When the epitope exists in cryptic form, it is not recognized by the given antibody, i.e., there is no binding of the antibody to this epitope or to the given cell at stage I. However, the epitope may be exposed by, e.g., undergoing modifications itself, or being unblocked because nearby or associated molecules are modified or because a region undergoes a conformational change. Examples of modifications include changes in folding, changes in post-translational modifications, changes in phospholipidation, changes in sulfation, changes in glycosylation, and the like. Such modifications may occur when the cell enters a different state, which may be called a second stage (stage II). Examples of second states, or stages, include activation, proliferation, transformation, or in a malignant status. Upon being modified, the epitope may then be exposed, and the antibody may bind.

Peptido-mimetics are small molecules, peptides, polypeptides, lipids, polysaccharides or conjugates thereof that have the same functional effect or activity of another entity such as an antibody.

### BRIEF DESCRIPTION OF THE DRAWINGS

FIG. 1 shows cleavage sites of endoprotease on the α chain of GPIb.

FIG. 2 depicts a Western blot showing binding of Y1 and Y17 to platelets in reduced and non-reduced conditions.

FIG. 3 is an outline of the optimal determinants for binding of Y1 to its epitope.

FIG. 4 depicts a Western blot demonstrating that cleavage of platelet GPIb by O-Sialoglycoprotein endoprotease abolishes binding of both Y1 and Y17.

FIG. 5 depicts a Western blot demonstrating that Y1 and Y17 bind similar glycocalicin fragments after cleavage by O-Sialoglycoprotein endoprotease.

FIG. 6 depicts the results of FACS analysis demonstrating that specific GPIb proteolysis abolishes Y1 binding to platelets.

FIG. 7 depicts a Western blot demonstrating that Y1 binds the N-terminal (His1-Glu282) fragment of platelet GPIbα after cleavage by mocarhagin.

FIG. 8 depicts a Western blot showing binding of Y1 and Y17 to glycocalicin after cleavage by mocarhagin.

FIG. 9 depicts a Western blot showing the binding of Y1 and Y17 to platelets.

FIG. 10 depicts a Western blot demonstrating that Y1 and Y17 bind glycocalicin similarly after cleavage by Ficin.

FIG. 11 depicts a Western blot demonstrating that Y1 reacts with the larger fragment generated by cathepsin G cleavage of GPIbaα.

FIG. 12 depicts a Western blot demonstrating that Y1 and Y17 react with the larger fragment generated by cathepsin G cleavage of GPIbaα.

FIG. 13 depicts a Western blot demonstrating that cleavage of glycocalicin by mocarhagin and cathepsin G abolishes binding of Y1.

FIG. 14 depicts a Western blot showing the binding of Y1 and Y17 to lysate of washed platelets cleaved by mocarhagin and cathepsin G.

FIG. 15 is a graph illustrating inhibition by Y1-scFv of agglutination of washed platelets.

FIG. 16 is a graph illustrating inhibition by Y1-scFv of aggregation of platelets in platelet-rich plasma.

FIG. 17 is a graph illustrating induction of agglutination of washed platelets by Y1-IgG.

FIG. 18 is a graph illustrating induction of platelet aggregation in platelet-rich-plasma by Y1-IgG.

FIG. 19 provides results of an ELISA assay.

FIG. 20 depicts a Western blot illustrating the specificity of binding of Y1 and α-CD42 (N1-19) to their ligands.

FIG. 21 depicts a Western blot Y1 reactivity with Y1-ligand on KG-1 cell membrane purified using immunoprecipitation and RP-HPLC.

FIG. 22 depicts a Western blot showing the effect of O-Sialoglycoprotein endopeptidase cleavage on Y1 binding.

FIG. 23 depicts a Western blot showing the effect after aryl-sulfatase cleavage on Y1 binding to RP-HPLC -purified KG-1 cell lysates, and heparin-BSA.

FIG. 24 depicts the immunoprecipitation scheme used in the analysis of the specificity of Y1 binding, the results of which are depicted in FIG. Tab 2A, page 17B.

FIG. 25 depicts Western blots comparing binding of Y1 and anti-CD-162 antibody to cells from AML patients and normal blood.

FIG. 26 depicts the results of a FACS analysis showing the ability of antibodies KPL1, PL1, and PL2 to compete with Y1 for binding.

FIG. 27 depicts the results of a FACS analysis demonstrating the specificity of Y1 binding.

FIG. 28 also depicts the results of a FACS analysis demonstrating the specificity of Y1 binding.

FIG. 29 is a graph illustrating % inhibition of Y1 binding in the presence of various peptides.

FIG. 30 is a graph depicting liver weights in mice in different treatment groups. FIG. 31 is a graph depicting % MOLT cells in bone marrow in mice in different treatment groups.

FIG. 32 is a graph depicting % MOLT cells in blood in mice in different treatment groups.

FIG. 33 is a graph depicting liver weights (mean +/− SEM) of mice at day 35.

FIG. 34 is a graph depicting liver weights (mean +/− SEM) of mice at day 35.

FIG. 35 is a graph illustrating effect of treatment on survival.

FIG. 36 is a graph depicting % occurrence of leukemia in different treatment groups.

FIG. 37 is a graph depicting % KG-1 cells in blood in different treatment groups.

FIG. 38 is a graph illustrating % KG-1 cells in bone marrow of experimental animals.

FIG. 39 is a graph illustrating the pharmakokinetics of TCA-precipitable radioactivity in plasma after intravenous injection of ¹²⁵I-CONY1 in mice. The sequence of CONY1 is presented at SEQ ID NO:204.

FIG. 40 is a graph illustrating the specific radioactivity of various organs/tissues after intravenous injection of ¹²⁵I-CONY1 in mice.

FIG. 41 is a graph illustrating the distribution of radioactivity of various organs/tissues after intravenous injection of ¹²⁵I-CONY1 in mice.

FIG. 42 is a graph of the Superdex 75 profile of Y1-cys-kak.

FIG. 43 reveals the size of the dimers compared to the monomer in reducing and non-reducing conditions.

FIG. 44 depicts a FACS analysis showing the level of binding of the IgG-Y1 molecule compared to that of scFv-Y1.

FIG. 45 depicts Western blots showing binding of Y1 and other antibodies to natural human platelet derived glycocalicin and to recombinant glycocalicin produced in E. coli.

FIG. 46 shows a binding comparison between a Y1 dimer, the Y1 scFv (CONY1), and Y1 IgG.

FIG. 47 shows a binding comparison between a Y1 sulfide bridge dimer with the Y1 scFv (CONY 1).

FIG. 48 provides the amino acid and nucleotide sequences of the heavy and light chains of Y1-IgG. The open reading frame (ORF) of the nucleotide sequence of Y1-HC (SEQ ID NO:205), the amino acid sequence of Y1-HC (SEQ ID NO:206), the ORF of the nucleotide sequence of Y1-LC (SEQ ID NO:207), and the amino acid sequence of Y1-LC (SEQ ID NO:208) are provided.

FIG. 49 provides the amino acid sequence of TM1 (SEQ ID NO:209).

FIG. 50 provides the amino acid and nucleotide sequences of the Y16 scFv (SEQ ID NO:210).

FIG. 51 provides the amino acid sequence of the Y1 Biotag (SEQ ID NO:211).

FIG. 52 provides the amino acid sequence of the Y1-cys-kak scFv (SEQ ID NO:. 212).

### DETAILED DESCRIPTION OF THE INVENTION

In the present invention, whole cells were used to select specific antibodies that recognize leukemia cell surface determinants, wherein the specific receptor was not previously known or characterized. Additionally, a multi-step biopanning process was utilized, in which phage were selected by panning on more than one cell type. This is a marked improvement over prior art methods in which the selection of antigen-specific phage antibodies has largely relied on biopanning against an immobilized single antigen, and there was only limited selection using whole cells as a target.

Certain epitopes that were identified by this multistep process are characterized by the presence of sulfated moieties, such as sulfated tyrosine residues or sulfated carbohydrate or lipid moieties, preferably within a cluster of two or more acidic amino acids, are found on ligands and receptors that play important roles in such diverse processes as inflammation, immune reactions, infection, autoimmune reactions, metastasis, adhesion, thrombosis and/or restenosis, cell rolling, and aggregation. Such epitopes are also found on diseased cells, such as B-CLL cells, AML cells, multiple myeloma cells, and metastatic cells. These epitopes are useful targets for the therapeutic mediation of these processes and for diagnostic procedures.

Although, these epitopes have variable primary amino acid sequences, antibodies directed against such sulfated epitopes are often capable of binding to, or crossreacting with, more than one such epitope on more than one molecule, albeit not necessarily simultaneously. Such antibodies are useful as therapeutic agents against cancers (both as anti-tumor agents and as anti-metastatic agents), leukemias, autoimmune diseases, viral diseases, diseases involving abnormal aggregation, diseases involving abnormal adhesion, infarction, cardiovascular diseases and inflammatory diseases.

The human scFv Y1 antibody was isolated from a human antibody phage display library that was used to screen fixed human platelets in order to identify antibodies that bind platelets. Several clones (different scFv antibodies) were isolated and characterized. One of these clones, designated as Y1, unexpectedly was found to bind leukemia cells derived from AML patients and patients having certain other leukemias. Another clone, Y17, was also isolated by panning on fixed platelets and was found to bind to human blood.

Proteins extracted from human platelets were Western blot analyzed on SDS-PAGE using the Y1 scFv antibody and the Y17 scFv antibody, in order to identify the receptors to which the antibodies bind on the surface of the platelets. Using this methodology, it was determined that the Y1 scFv and Y17 scFv epitope on platelets is glycocalicin, one of the subunits of the CD42 complex.

The human platelet derived glycocalicin extracellular fragment was purified from activated platelets. It was digested with various proteases, such as ficin, mocarhagin, cathepsin G, in order to localize precisely the Y1 binding epitope on the glycocalicin molecule. Analysis was performed by the Western blot methodology using the Y1 antibody as a detection tool. In addition, commercially available anti-glycocalicin antibodies (antibodies that are known to bind to different epitopes of glycocalicin) were used in a competition binding assay with the Y1 antibody to determine the Y1 binding epitope on glycocalicin.

Based on the results, it was concluded that amino acids 272 through 285 of glycocalicin play a major role in the binding of Y1 to glycocalicin. In addition, since the E. coli derived recombinant N-terminal polypeptide of glycocalicin (amino acids 1 to 340 and 1 to 480) was not detectable by the Y1 antibody, it was concluded that Y1 binding to its epitope depends on post-translational modifications, such as glycosylation or sulfation, which are modifications that are not known to occur in E. coli).

In order to verify this hypothesis, the purified glycocalicin was treated with enzymes (glycosidases) that remove N and O-linked sugar moieties from proteins and enzymes (sulfatases) that remove sulfate moieties from proteins. The binding of the Y1 antibody to glycocalicin or glycocalicin derived fragments was not affected by the glycosidases. This result indicates that sulfated groups are essential for the binding of Y1 to glycocalicin.

In order to further verify these results, sulfated and non-sulfated synthetic peptides based on the identified epitope (amino acids 272 to 285 of glycocalicin) were prepared and used to assess the binding specificity of theY1 antibody to glycocalicin in their presence (ELISA assay). Sulfated peptides inhibited the binding of the Y1 antibody to glycocalicin several folds higher than the related non-sulfated peptides indicating that sulfation is required for binding.

From the above experimental results, it was concluded that the epitope for Y1 antibody is located between amino acids 272 and 285 on glycocalicin in which there is cluster of negatively charged amino acids.

In parallel, the binding of the Y1 antibody to KG-1 cells (a human cell line derived from AML patient), to various human plasma derived proteins, and to primary leukemia patient blood samples was studied.

The Y1 antibody was found to bind with relatively low affinity to two human plasma derived proteins, one in the size of ˜50kD molecular weight, which was identified as fibrinogen γ prime and a ˜80kD molecular weight protein, which was identified as complement compound 4 (CC4) and human lumican. These proteins contain sulfated tyrosine residues accompanied by a stretch of negatively charged amino acids.

The Y1 ligand on KG-1 cells was identified as PSGL-1, which is a receptor for E, L- and P-selectins. PSGL-1 was identified as the ligand of the Y1 antibody on KG-1 cells based on competition assays (wherein binding of the Y1 antibody to the KG-1 cells was carried out in the presence of different commercially available anti PSGL-1 antibodies) and upon a set of experiments using sulfated and non-sulfated synthetic peptides derived from the N-terminal site of PSGL-1. The N-terminal site of PSGL-1 contains sulfated tyrosine residues accompanied by a cluster of negatively charged amino acids.

Although the Y1 antibody binds to several molecules, such as the glycocalicin molecule on platelets, fibrinogen-gamma prime, the complement compound 4 of human plasma, and the PSGL-1 molecule on KG-1 cells, its affinity to primary leukemia cells derived from either AML or multiple myeloma (MM) patients is several magnitudes higher relative to the previously mentioned epitopes. Moreover, the fact that commercially available anti PSGL-1 antibody (KPL1) does not recognize all (7 out of 12) diseased primary leukemia cells in blood samples derived from patients, while the Y1 antibody recognizes them specifically and selectively, indicates that there are additional epitopes for Y1 antibody on primary leukemia cells that differ from that on KG-1 cells.

Examples of sulfated epitopes according to the present invention include those delineated in Formulae I, II, and III, as well as derivatives, homologs, mimetics, and variants thereof.

Formula (I):

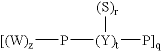

Wherein:

W is any amino acid other than Aspartate and Glutamate

Y is any naturally occurring moiety that is capable of being sulfated

P is (A)ₘ(A)ₙ(X)ᵤ or (X)ᵤ(A)ₙ(A)ₘ or (A)ₙ(X)ᵤ(A)ₘ or (A)ₙ(A)ₘ(X)ᵤ or (X)ᵤ(A)ₘ(A)ₙ or (A)ₘ(X)ᵤ(A)ₙ

S is sulfate or a sulfated molecule

X is any amino acid except Aspartate, Glutamate, or Tyrosine

A is any negatively charged amino acid or leucine, isoleucine, proline, phenylalanine, serine, or glycine

q is 1 to 6

z is 0, 1, or 2

r is 0 or 1

t is 1, 2 or 3

u is 0 to 2

n is 0 to 3

m is 0 to 3

wherein if n=0 then m>0 ; wherein if m=0 then n>0; wherein if q is 1, r is 1, and if q is >1 at least one of Y is sulfated; and further wherein the isolated epitope is capable of being bound by an antibody, antigen-binding fragment thereof, or complex thereof comprising an antibody or binding fragment thereof, comprising a first hypervariable region comprising SEQ ID NO:8 or SEQ ID NO:20.

A preferred epitope is the epitope of Formula I wherein W is Glycine, Y is a peptido conjugate of Tyrosine or a glyco conjugate of Asparagine, Serine or Threonine; A is Glutamate, γ Carboxy Glutamate or Aspartate; and q is 1, 2, or 3. In certain embodiments, Y is a peptido conjugate of Tyrosine; q is 3; and r is 1.

Formula (II):

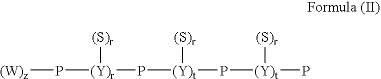

Wherein:

W is any amino acid other than Aspartate and Glutamate

Y is any naturally occurring moiety that is capable of being sulfated

P is (A)ₘ(A)ₙ(X)ᵤ or (X)ᵤ(A)ₙ(A)ₘ or (A)ₙ(X)ᵤ(A)ₘ or (A)ₙ(A)ₘ(X)ᵤ or (X)ᵤ(A)ₘ(A)ₙ or (A)ₘ(X)ᵤ(A)ₙ

S is a sulfate or a sulfated molecule

X is any amino acid except Aspartate, Glutamate or Tyrosine

A is any negatively charged amino acid or leucine, isoleucine, proline, phenylalanine, serine, or glycine

z is 0, 1,or 2

r is 0 or 1

t is 1, 2 or 3

u is 0 to 2

n is 0 to 3

m is 0 to 3

wherein if n=0 then m>0; wherein if m=0 then n>0; wherein at least one Y is sulfated; and further wherein the isolated epitope is capable of being bound by an antibody, antigen-binding fragment thereof, or complex thereof comprising an antibody or binding fragment thereof, comprising a first hypervariable region comprising SEQ ID NO:8 or SEQ ID NO:20.

A preferred epitope is the epitope of Formula II wherein: W is Glycine; Y is a peptide conjugate of Tyrosine or a glyco conjugate of Asparagine, Serine or Threonine; A is Glutamate, γ Carboxy Glutamate or Aspartate, Leucine, Isoleucine Phenylalanine, Serine or Glycine. In certain embodiments, Y is a peptido conjugate of Tyrosine; q is 3; and r is 1.

Formula (III):

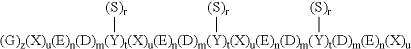

Wherein:

G is Glycine

B is Glutamate

D is Aspartate

Y is Tyrosine

S is sulfate or a sulfated molecule

X is any amino acid except the above

z is 0, 1, or 2

t is 1, 2 or 3

r is 0 or 1

u is 0 to 2

n is 0 to 3

m is 0 to 3

wherein at least one Y is sulfated; wherein if n=0 then m>0; wherein if m=0 then n>0; and further wherein the isolated epitope is capable of being bound by an antibody, antigen-binding fragment thereof, or complex thereof comprising an antibody or binding fragment thereof, comprising a first hypervariable region comprising SEQ ID NO:8 or SEQ ID NO:20.

A preferred epitope is the epitope of Formula III wherein r is 1.

The sulfated moiety of any of the Formulae may be also a peptido- or glyco- or lipo-conjugate. Y may comprise a lipid and/or carbohydrate molecule. The epitopes may have at least one post-translational modification in addition to sulfation.

Such epitopes are found on such diverse molecules as GPIb and PSGL-1 and are found on certain diseased cells, such as B-CLL cells, AML cells, multiple myeloma cells, and metastatic cells. Sulfation of tyrosine and/or other moieties is particularly important for binding to these epitopes. Human proteins known to be tyrosine sulfated include the following:

!Peptide? Sequence

| | |
|---|---|
| Thrombomodulin (408-426) | E C P E G Y I L D D G F I C T D I D E |
| Human GPIbα (269-287) | D E G D T D L Y D Y Y P E E D T E G D |
| Human Heparin Cofactor II (56-75) | G E E D D D Y L D L E E D D D Y I D I V D |
| Human Fibrinogen γ′ (408-427) | V R P E H P A E T E Y D S L Y P E D O L |
| α-2-Antiplasmin | P P M E E D Y P Q F G S P |
| Cholecystokinin (CCK) | R I S D R D Y M G W M D F |
| α-2-Choriogonadotropin | C H C S T C Y Y H K S - C O O H |
| Complement C4 | M E A N E D Y E D Y E Y D E L P A K |
| PSGL-1 | Q A T E Y E Y L D Y D F L P E T |
| Factor VIII (716-731) | G D Y Y E D S Y E D I S A Y L L |
| Lumican | GYYDYDFPL |

Y1—Production and Selection

One example of an antibody of the present invention that binds to epitopes of Formulae I-III is the fully human monoclonal antibody Y1. The selection, production, and initial characterization of Y1 are described in detail in U.S. patent application Ser. Nos. 09/751,181 and 60/258,948. Briefly, a phage display library displaying scFv antibody fragments was utilized to obtain and produce targeting molecules, and flow cytometry, particularly fluorescence-activated cell sorting (FACS), was used for identifying and isolating specific phage clones, the peptide or polypeptide of which recognizes target cells. The phage display library used herein was constructed from peripheral blood lymphocytes of a non-immunized human donor.

Phage clones were selected by and identified through a multi-step procedure known as biopanning. Biopanning was carried out by incubating phage displaying protein ligand variants (a phage display library) with a target, removing unbound phage by a washing technique, and specifically eluting the bound phage. The eluted phage were optionally amplified before being taken through additional cycles of binding and optional amplification which enriched the pool of specific sequences in favor of those phage clones bearing antibody fragments that display the best binding to the target. After several rounds, individual phage clones were characterized, and the sequences of the peptides displayed by the clones were determined by sequencing the corresponding DNA of the phage virion.

In the present invention, screening of platelets was carried out against non-defined epitopes for the initial biopanning steps, with subsequent clone selection performed with a desired target cell (e.g., B-CLL cells, AML cells, multiple myeloma cells, and metastatic cells), the targeted cell surface markers of which are unknown.

Malignant and diseased blood cells (e.g., leukemia or lymphoma) are characterized as immature cells that express cell surface proteins normally found in partially differentiated hematopoietic progenitors. Thus, platelets are an attractive source for the identification of premature cell surface markers expressed on diseased or malignant blood cells.

Y1, an scFv clone which binds to platelets and myleogenous leukemia cells, particularly AML cells, was selected. Y1 scFv has the sequence SEQ ID NO:25. The binding characteristics of Y1 are primarily attributable to its heavy chain CDR3 region, which has the sequence SEQ ID NO:8. Full Y1-IgG antibodies were also produced.

A second scFv clone, Y17, which binds to platelets and cell lines derived from human myleogenous leukemia cells, particularly AML cells, was also selected. Y17 scFv has the sequence SEQ ID NO:203. The binding characteristics of Y17 are primarily attributable to its heavy chain CDR3 region, which has the sequence SEQ ID NO:20. Full Y17-IgG antibodies were also produced.

Antibody Production

CDRs according to the present invention may also be inserted into cassettes to produces antibodies. A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed or attached at one or both ends.

The amino acid sequence of the cassette may ostensibly be fixed, whereas the replaced, inserted or attached sequence can be highly variable. The cassette can be comprised of several domains, each of which encompasses a function crucial to the final construct.

The hypervariable regions of antibodies of the invention form the antigen binding sites of antibodies of the present invention. The antigen-binding site is complementary to the structure of the epitopes to which the antibodies bind and therefore are referred to as complementarity-determining regions (CDRs). There are three CDRs on each light and heavy chain of an antibody, each located on the loops that connect the β strands of the V_{H} and V_{L} domains.

The cassette of a particular embodiment of the present invention comprises, from the N-terminus, framework region 1 (FR1), CDR1, framework region 2 (FR2), CDR2, and framework region 3 (FR3).

In an embodiment of the invention, it is possible to replace distinct regions within the cassette. For example, the CDR and CDR1 hypervariable regions of the cassette may be replaced or modified by non-conservative or, preferably, conservative amino acid substitutions. More specifically, the CDR2 and CDR1 regions of a cassette of consecutive amino acids selected from the group comprising of SEQ ID NOS:30-113 or a fragment thereof can be replaced by SEQ ID NOS:115 and 114, respectively. Even more specifically, the CDR2 and CDR1 regions of a cassette of consecutive amino acids selected from the group comprising of SEQ ID NOS:30-32, 35, 37-39, 41, 43, 45, 46, 48, 51, 54, 57, 59-68, 70, 71, 76-85, 87, 89-92, 94, 97, 99, 103, 106, 112, and 113 or fragment thereof can be replaced by SEQ ID NOS:115 and 114, respectively.

In a preferred embodiment of the invention, the peptide or polypeptide comprises a heavy and a light chain, and each chain comprises a first, second and third hypervariable region which are the CDR3, CDR2 and CDR1 regions, respectively. The binding selectivity and specificity are determined particularly by the CDR3 region of a chain, possibly by the CDR3 region of the light chain and, preferably, by the CDR3 region of the heavy chain, and secondarily by the CDR2 and CDR1 regions of the light chain and, preferably, of the heavy chain. The binding selectivity and specificity may also be secondarily influenced by the upstream or downstream regions flanking the first, second, and/or third hypervariable regions.

In a preferred embodiment, the CDR3 region of the peptide or polypeptide has an amino acid sequence selected from the group comprising SEQ ID NOS:8-24.

In a more preferred embodiment, the CDR3 region of the heavy chain has an amino acid sequence selected from the group comprising SEQ ID NOS:8-24, the CDR2 has an amino acid sequence identical to SEQ ID NO:115, and the CDR1 region has an amino acid sequence identical to SEQ ID NO:114.

In a most preferred embodiment of the invention, the CDR3 region has an amino acid sequence identical to SEQ ID NO:8.

A preferred embodiment of the invention is a scFv with a CDR3 sequence identical to SEQ ID NO:8 and a full scFv sequence identical to SEQ ID NO:25.

In a most preferred embodiment of the invention the CDR3, CDR2 and CDR1 regions have the amino acid SEQ ID NOS:8, 115 and 114, respectively.

In an embodiment of the invention, the Fv peptide comprises a CDR1 and CDR2 region of the variable heavy chain which itself comprises a cassette with an amino acid sequence selected from the group comprising SEQ ID NOS:30-113; a CDR3 region, preferably of the variable heavy chain, which has an amino acid sequence selected from the group comprising SEQ ID NO:8-24; an upstream region flanking the CDR3 region which has the amino acid sequence of SEQ ID NO:117; a downstream region flanking the CDR3 region which has the amino acid sequence of SEQ ID NO:116; a spacer of 0-20 amino acid residues of SEQ ID NO:123 or 124; a variable light chain region the sequence of which is SEQ ID NO:7.

Similarly, in another embodiment the upstream region flanking the CDR2 region has the amino acid sequence of SEQ ID NO:119, the downstream region flanking the CDR2 region has the amino acid sequence of SEQ ID NO:118, the upstream region flanking the CDR1 region has the amino acid sequence of SEQ ID NO:121 and the downstream region flanking the CDR1 region has the amino acid sequence of SEQ ID NO:120.

A preferred embodiment of the invention provides for a peptide or polypeptide wherein the second and third hypervariable regions are a CDR2 and a CDR1 hypervariable region, respectively and wherein the CDR3 amino acid sequence is SEQ ID NO:8, wherein the CDR2 amino acid sequence is SEQ ID NO:115, wherein the CDR1 amino acid sequence is SEQ ID NO:114, wherein the upstream region flanking the CDR3 region has the amino acid sequence of SEQ ID NO:117, wherein the downstream region flanking the CDR3 region has the amino acid sequence of SEQ ID NO:116, wherein the upstream region flanking the CDR2 region has the amino acid sequence of SEQ ID NO:119, wherein the downstream region flanking the CDR2 region has the amino acid sequence of SEQ ID NO:118, wherein the upstream region flanking the CDR1 region has the amino acid sequence of SEQ ID NO:121 and wherein the downstream region flanking the CDR1 region has the amino acid sequence of SEQ ID NO:120.

Another preferred embodiment of the invention provides for an Fv molecule that comprises a first chain having a first, a second and a third hypervariable region and a second chain having a first, a second and a third hypervariable region, wherein one of the hypervariable regions of the first chain has a sequence selected from the group consisting of SEQ ID NOS:8-24, and wherein one of the hypervariable regions of the second chain has a sequence selected from the group consisting of SEQ ID NOS:1-6 and 125-202, and wherein the first, second and third hypervariable regions are a CDR3, CDR2 and CDR1 region, respectively and wherein the Fv is a scFv or a dsFv, and optionally having one or more tags.

Another embodiment of the invention provides for a peptide or polypeptide (i) wherein the first chain and the second chain each comprises a first hypervariable region selected from the group consisting of SEQ ID NOS:8-24; or (ii) wherein the first hypervariable region of the first and second chains are identical and selected from the group consisting of SEQ ID NOS:8-24; or (iii) wherein the first hypervariable region of the first chain is selected from the group consisting of SEQ ID NOs:8-24, and the first hypervariable region of the second chain is selected from the group consisting of SEQ ID NOS:1-6 and 125-202; or (iv) wherein the first hypervariable region of the first chain is selected from the group consisting of SEQ ID NOS:1-6 and 125-202, and the first hypervariable region of the second chain is selected from the group consisting of SEQ ID NOS:8-24.

A further embodiment provides for the peptide or polypeptide of the invention wherein the second and third hypervariable regions of the first chain are SEQ ID NOs:114 and 115, respectively.

For all the amino acid sequences of ≦25 amino acid residues described and detailed herein (e.g., CDR regions, CDR flanking regions), it is to be understood and considered as a further embodiment of the invention that these amino acid sequences include within their scope one or two amino acid substitution(s) and that preferably the substitutions are conservative amino acid substitutions. For all the amino acid sequences of >25 amino acid residues described and detailed herein, it is to be understood and considered as an embodiment of the invention that these amino acid sequences include within their scope an amino acid sequence with ≧90% sequence similarity to the original sequence (Altschul et al., Nucleic Acids Res., 25, 3389-3402 (1997)). Similar or homologous amino acids are defined as non-identical amino acids which display similar properties, e.g., acidic, basic, aromatic, size, positively or negatively charged, polar, non-polar.

Percentage amino acid similarity or homology or sequence similarity is determined by comparing the amino acid sequences of two different peptides or polypeptides. The two sequences are aligned, usually by use of one of a variety of computer programs designed for the purpose, and amino acid residues at each position are compared. Amino acid identity or homology is then determined. An algorithm is then applied to determine the percentage amino acid similarity. It is generally preferable to compare amino acid sequences, due to the greatly increased sensitivity to detection of subtle relationships between the peptide, polypeptide or protein molecules. Protein comparison can take into account the presence of conservative amino acid substitutions, whereby a mismatch may yet yield a positive score if the non-identical amino acid has similar physical and/or chemical properties (Altschul et al., Nucleic Acids Res., 25, 3389-3402 (1997).

In an embodiment of the invention the three hypervariable regions of each of the light and heavy chains can be interchanged between the two chains and among the three hypervariable sites within and/or between chains.

Polyclonal Antibodies Against V_{L} (Derived from Y1)

The DNA fragment encoding the V_{L} domain (variable light chain) of human antibody was PCR-cloned from the Y1 clone (the identical DNA fragment can be obtained from any other clone in the Nissim I library (Nissim et al., “Antibody fragments from a ‘single pot’ phage display library as immunochemical reagents,” EMBO J. 13(3): 692-698 (1994)) or even from the human genome using the same methodology) with the following synthetic oligonucleotide primers: oligo 5′-Nde1 (TTTCATATGGAGCTGACTCAGGACCCTGCT) and oligo 3′-EcoRI (TTTGAATTCCTATTTTGCTTTTGCGGC). After amplification by polymerase chain reaction (PCR conditions: 94° 1′, 56° 2′, 72° 2′×30 then 65° 5′) the obtained DNA fragment was digested with NdeI and EcoRI restriction enzymes and cloned into NdeI and EcoRI restriction enzymes sites of a pre-digested plasmid, which is an IPTG inducible expression vector used for prokaryotic expression of recombinant proteins in E. coli. E. coli cells were transformed with the ligation mixture and positive clones were selected by PCR amplification using the above oligonucleotide primers. Cells harboring this plasmid were grown and induced for expression by IPTG. Bacterial cells were harvested by centrifugation from 1 liter of culture post induction with IPTG, inclusion bodies were isolated and solubilized in guanidine-HCl+DTE, and refolded by dilution in a buffer containing TRIS-ARGININE-EDTA. After refolding at 5-10° for 48 hrs, the solution containing protein was dialyzed and concentrated to 20 mM Glycine pH 9. The dialyzed solution containing proteins was re-purified by using an ionic exchange column, HiTrapQ, and eluted with a gradient of NaCl. The main peak was analyzed by SDS-PAGE and by gel filtration. At least 10 mgs of purified V_{L} were obtained from an original 1 liter culture.

Rabbits were immunized with V_{L} (400 mg) in the presence of CFA (complete Fruend's adjuvant) then by V_{L} (200 mg) in the presence of IFA (incomplete Fruend's adjuvant) at 2 to 4 weeks intervals. The titers obtained were low (1:50-1:100) probably due to the high homology between the V_{L}'s from human and rabbit.

Polyclonal Antibodies Against scFv Antibodies

Two individual scFv antibody clones (Y1 and N14 ) derived from the Nissim I antibody phage display library (Nissim et al., “Antibody fragments from a ‘single pot’ phage display library as immunochemical reagents,” EMBO J. 13(3): 692-698 (1994))were cultured separately. After IPTG induction the cultures were grown at 22° C. for 16 hours. The scFv antibody fragments were harvested from the bacterial cell periplasm and were purified on a Protein A-Sepharose column. All the procedures for bacterial clone culturing, induction protocol, scFv antibody fragment harvesting and antibody fragment purification were carried out in accordance with: Harrison J. L., Williams S. C., Winter G, and Nissim A. Methods Enzymol. 267: 83-109 (1996). Basically, any two or more individual scFv clones can be selected from the Nissim I antibody phage display library in order to prepare rabbit derived polyclonal antibodies that recognize any individual scFv antibody that is present in the Nissim library or any IgG or fragment thereof provided that it contains the same V_{L} or a fragment thereof.

Rabbits were immunized with 400 mg of 1:1 ratio mixture of the purified scFv antibody fragments in the presence of complete Fruend's adjuvant then with 200 mg of that mixture in the presence of incomplete Fruend's adjuvant, at 2 to 4 weeks intervals.

For detection of the scFv antibodies binding to cells by flow cytometry (FACS) or to various protein fractions on SDS-PAGE ( Western blot analysis), the polyclonal anti scFv antibodies were used directly from the serum of the immunized rabbits or after purification on a Protein A-Sepharose column.

Characterization of Y1 Binding Site on Platelets

Circulating platelets are cytoplasmic particles released from the periphery of megakaryocytes. Platelets play an important role in hemostasis. Upon vascular injury, platelets adhere to damaged tissue surfaces and attach one another (cohesion). This sequence of events occurs rapidly, forming a structureless mass (commonly called a platelet plug or thrombus) at the site of vascular injury. The cohesion phenomenon, also known as aggregation, may be initiated in vitro by a variety of substances, or agonists, such as: collagen, adenosine-diphosphate (ADP), epinephrine, serotonin, and ristocetin. Aggregation is one of the numerous in vitro tests performed as a measure of platelet function.

Several lines of evidence in the prior art indicate that the cluster of negatively charged amino acids between Asp269 and Asp287 of GPIbα is important for von Willebrand Factor (vWF) binding to platelets, which in turn mediates platelet adhesion to damaged blood vessels, platelet aggregation induced by high shear in regions of arterial stenosis, and platelet activation induced by low concentrations of thrombin. Ward, C. M., et al., Biochemistry 35(15): 4929-39 (1996). The interaction of vWF with GPIb is dependent upon an activation event or conformational change in vWF structure when bound to matrix or exposed to shear. This process is mimicked in vitro by specific modulators that bind to vWF, such as ristocetin and botrocetin.

Reactivity of Y1 to Platelet Cell Extract

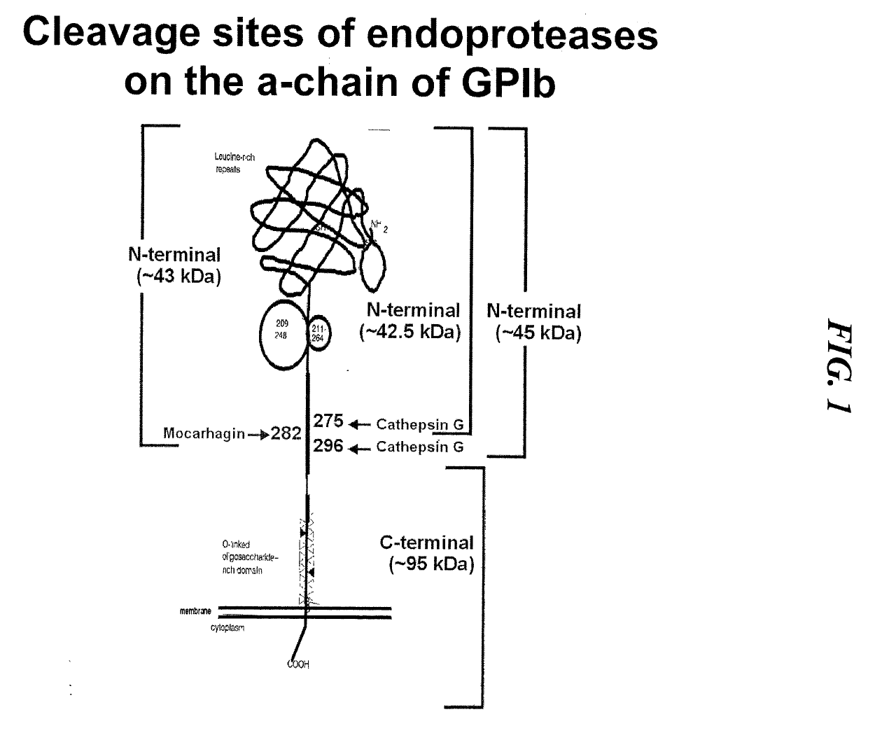

Immunoblotting and endoprotease cleavage techniques were used to identify the epitope for Y1 on the surface membrane of platelets. Endoprotease cleavage sites on the GPIbα molecule are shown in FIG. 1.

Western Blot Analysis

Y1 scFv was selected from phage antibody library by biopanning on human platelets and was found to bind to fixed and washed human platelets. Characterization of Y1 was done by using ELISA assay and by FACS analysis.

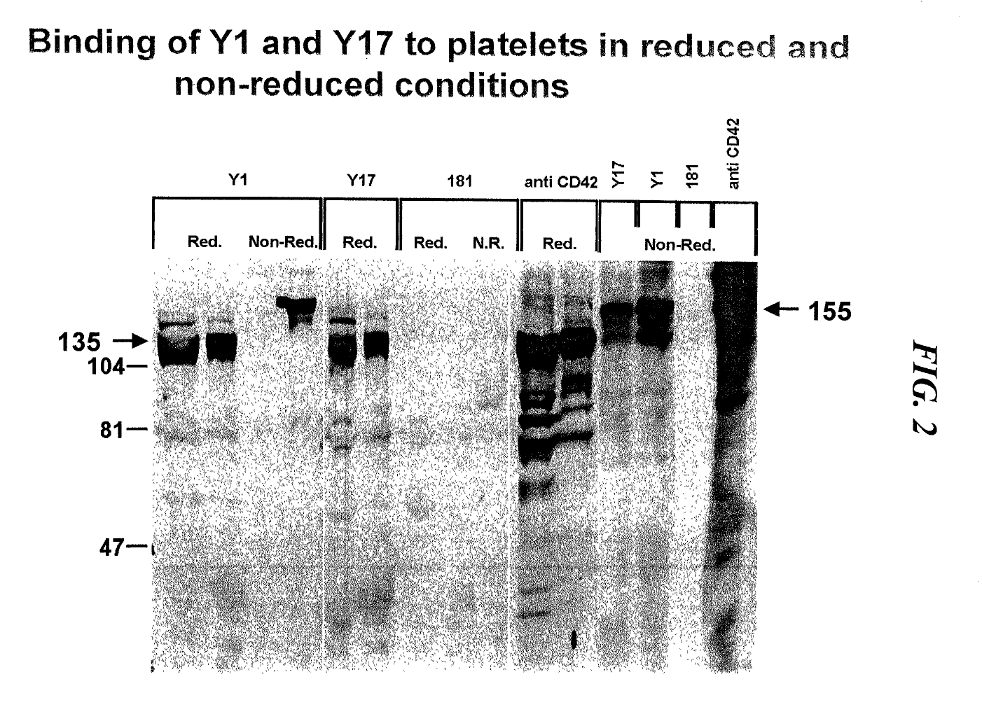

In order to characterize the epitope on the platelet membrane to which Y1 binds, platelet surface proteins were separated by SDS-PAGE (under both reducing and non-reducing conditions) and immunoblotted with biotin labeled-Y1 under reducing and non-reducing conditions. The results of this experiment demonstrate that Y1 reacts with a protein with a molecular mass of 135 kDa under reducing conditions, and with a protein with molecular mass of ˜160 kDa under non-reducing conditions. These molecular masses correspond to platelet GPIbα, which has a molecular mass of 135 kDa under reducing conditions. Under non-reducing conditions, the GPIbα chain disulfide-linked to GPIbβ has a molecular mass of 160-kDa. (FIG. 2).

The GPIba chain is disulfide-linked to the GPIbβ chain to form the platelet membrane protein GPIb. Monoclonal antibodies, MCA466S (Serotec) and S.C.7071(Santa Cruz), are known to bind respectively to the C-terminal fragment of GPIbα and to the N-terminal of GPIbα and were found to react to the same fragments with which Y1 reacts under reducing and non-reducing conditions (the S.C. was used only under reducing conditions). These results further confirm that Y1 binds to the GPIbα platelet surface protein.

Further analysis on semipurified GPIb fragment (glycocalicin) by Western analysis confirmed that indeed Y1 binds to the alpha subunit of the GPIb complex.

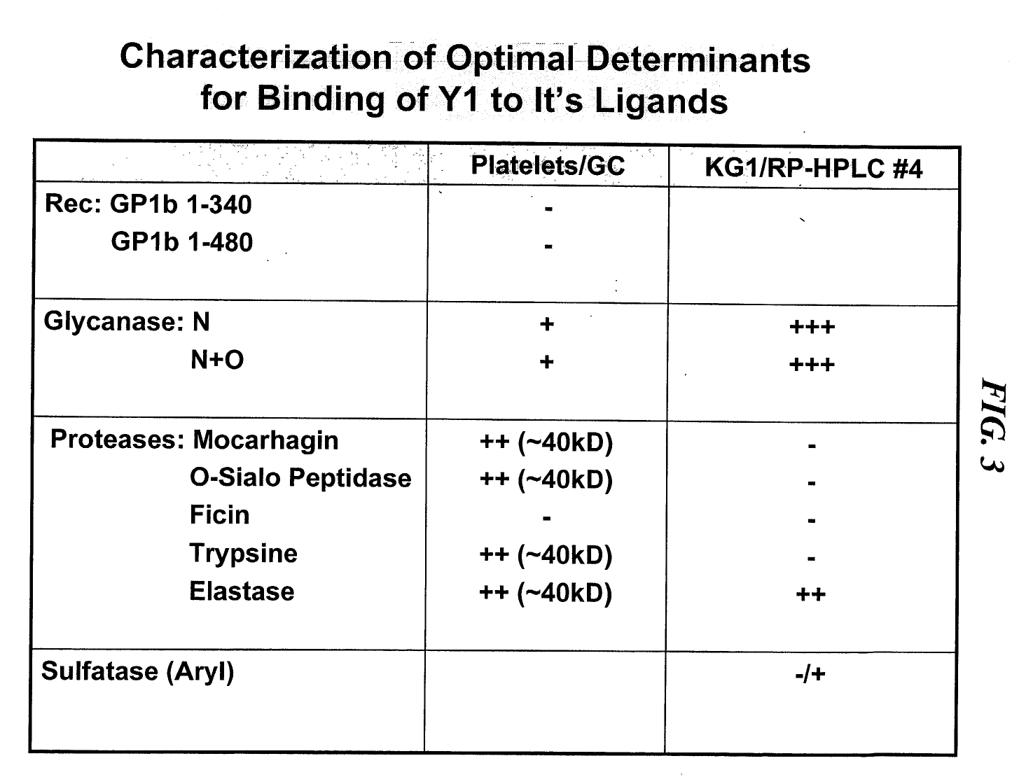

Western analysis of recombinant GPIb expressed in E. coli demonstrated that GPIb expressed in E. coli does not react with Y1. Thus, it appears that post-translational modification, which does not occur in E. coli is required for Y1 binding. Neither N- nor O-glycanases affect the binding of Y1 to KG-1 cells. However, Y1 binding can be inactivated by treatment of ligands with aryl sulfatases or by proteases. (FIG. 3).

Localization of Y1 Epitope Site on GPIbα Fragment of Glycocalicin (GC)

To further localize the Y1 binding site, specific endoproteases with known cleavage sites were used to digest GPIb and the fragments were tested for Y1 binding.

Effect of O-Sialoglycoprotein Endoprotease on Y1 Binding to Platelet GPIbα

The enzyme O-Sialoglycoprotein endoprotease from Pasteurella haemolytica (Cedarlan CLE 100) selectively cleaves human platelet GPIb and specifically cleaves only proteins containing sialylated, O-linked glycans. O-Sialoglycoprotein endoprotease does not cleave N-linked glycoproteins or unglycosylated proteins. This enzyme has been reported to cleave GPIb, which is heavily O-glycosylated, but not GPIIb-IIIa or other receptors on platelets. GPIbα was digested with O-Sialoglycoprotein endoprotease in order to further establish the binding of Y1 to the molecule.

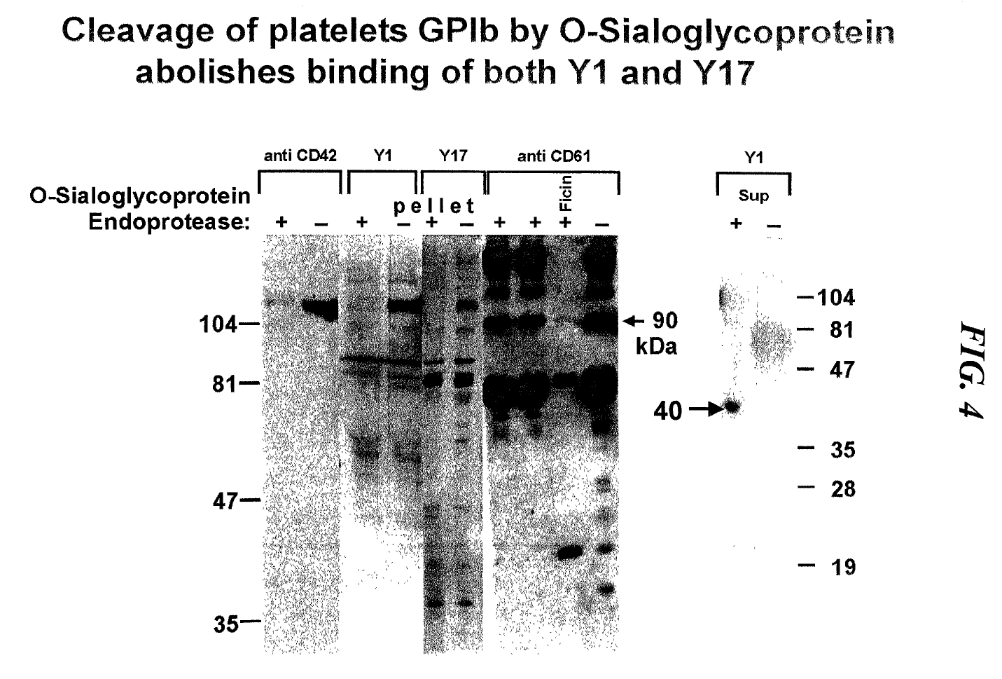

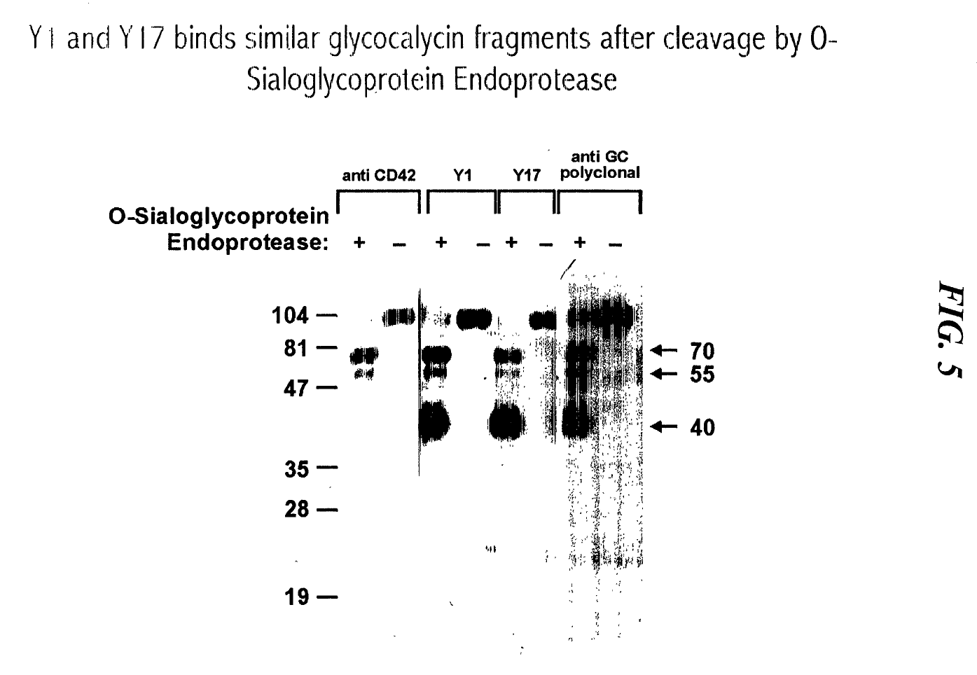

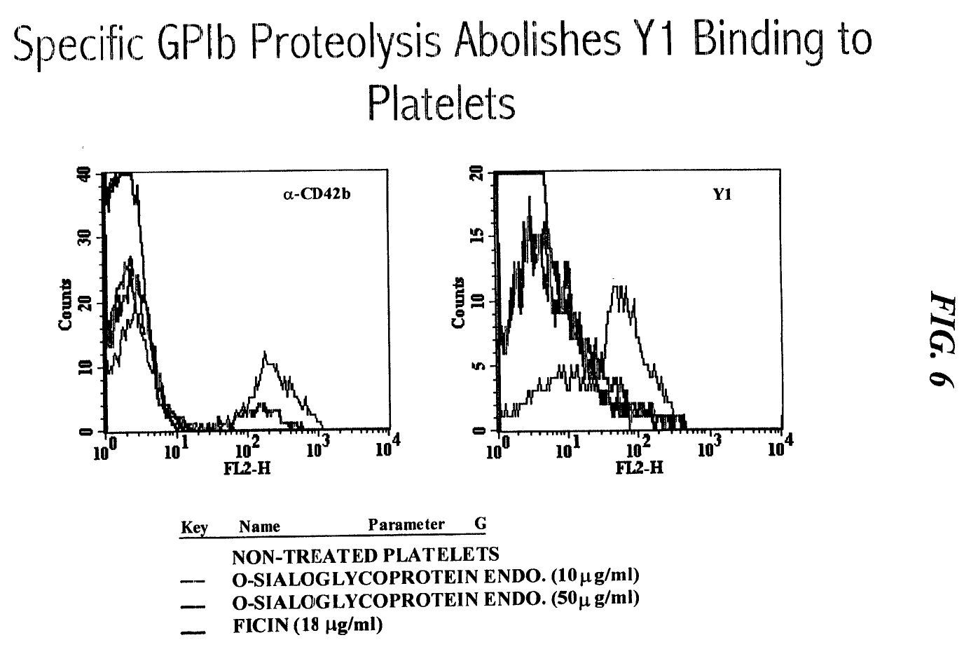

Immunoblots (FIGS. 4 and 5) and FACS analysis (FIG. 6) demonstrated that incubation of washed platelets with O-Sialoglycoprotein endoprotease abolishes binding of Y1, as well as the binding of monoclonal antibody MCA466S (Serotec), which is directed against GPIbα. The endoprotease did not alter the binding of a monoclonal antibody (anti-CD61) directed against GPIIb/IIIa. (FIG. 4). These results provide additional evidence that the receptor for Y1 on platelet membranes is GPIbα.

Mocarhagin Cleavage of GPIb—Mapping of the Y1 Epitope

Mocarhagin [Sigma L4515a] is a cobra venom metalloproteinase that cleaves platelet GPIbα specifically at a single site between residues glu-282 and asp-283, thereby generating two stable products: a ˜45-kDa N-terminal fragment (His1-Glu282), which is released into the supernatant, and a membrane-bound ˜95 kDa C-terminal fragment.

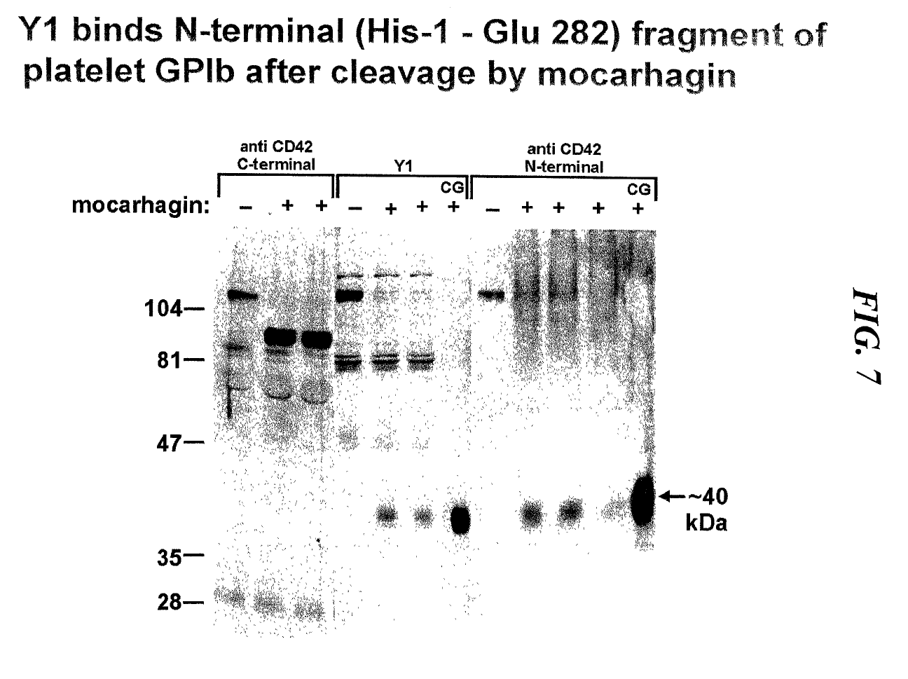

Washed platelets were treated by mocarhagin, and platelet lysates were electrophoresed on SDS-polyacrylamide gels and transferred to nitrocellulose. Western blot analysis of lysates of mocarhagin-treated washed platelets with Y1 shows a loss of the band corresponding to GPIbα (135 kDa) and binding of Y1 to the N-terminal ˜45 kDa tryptic fragment. A monoclonal antibody (MCA466S) directed against the C-terminal fragment of GPIba reacted with the ˜95 kDa C-terminal fragment, and a monoclonal antibody (S.C.7071) directed against the N-terminal fragment of GPIbα reacted with the same ˜45 kDa fragment that was recognized by Y1. (FIG. 7).

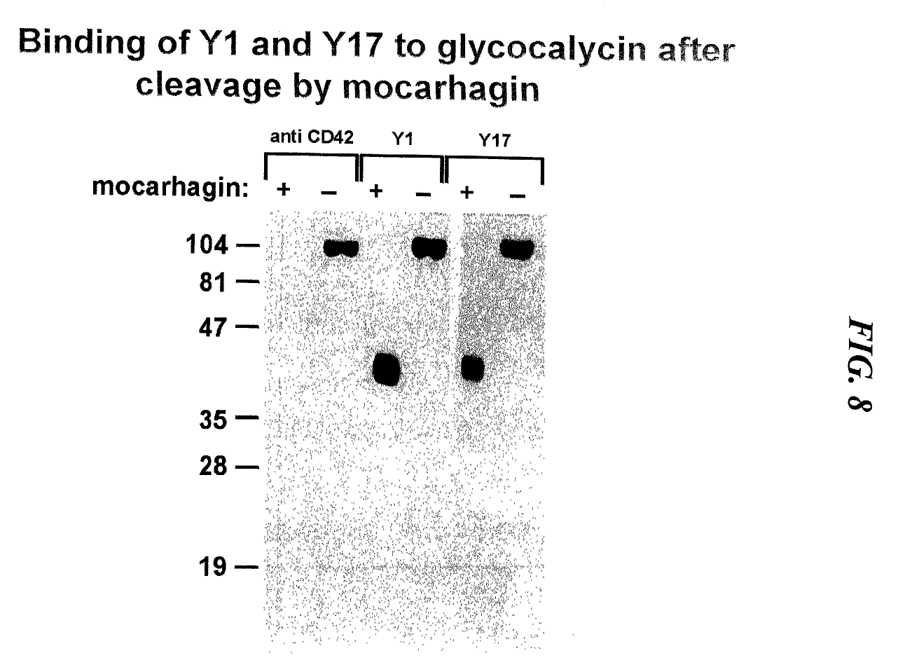

Mocarhagin treatment of glycocalicin (soluble, extracellular fragment of GPIbα) gave similar results to those observed with washed platelets, showing binding of Y1 and monoclonal antibody S.C.7071 to the ˜45 kDa N-terminal cleavage product fragment of GPIbα. (FIG. 8). These results suggest that the epitope for Y1 is contained within the sequence His1-Glu282.

Characterization of the Y17 Clone—Binding to GPIb

Y17, a second scFv human antibody fragment of the invention, which was selected in the same manner as Y1, was characterized using the methods used to characterize Y1. [See Example 17] Briefly, Y17 was selected from a phage antibody library by biopanning on human platelets. Characterization of Y17 was done by using ELISA assay and FACS analysis. Y17 was found to bind to both fixed and washed human platelets. In order to further characterize the receptor on platelet membranes which bind Y17, platelet proteins were separated by SDS-PAGE and immunoblotted with biotin labeled-Y17 under reducing and non-reducing conditions. The results demonstrated that Y17 reacts with protein having an apparent molecular weight of 135 kDa under reducing conditions, and with a protein having an apparent molecular weight of ˜160 kDa under non-reducing conditions. These results correspond to platelet GPIbα which under reducing conditions has a molecular weight of 135 kDa and under non-reducing conditions has a molecular weight of 160 kDa and consists of the GPIbα-chain disulfide linked to GPIbα. Monoclonal antibodies, MCA466S (Serotec) directed against the C-terminal fragment of GPIbα monoclonal antibody S.C. 7071 (Santa Cruz) that recognize the N-terminus of GPIbα react with the same bands as Y17 under reducing and non-reducing conditions. (FIG. 2).

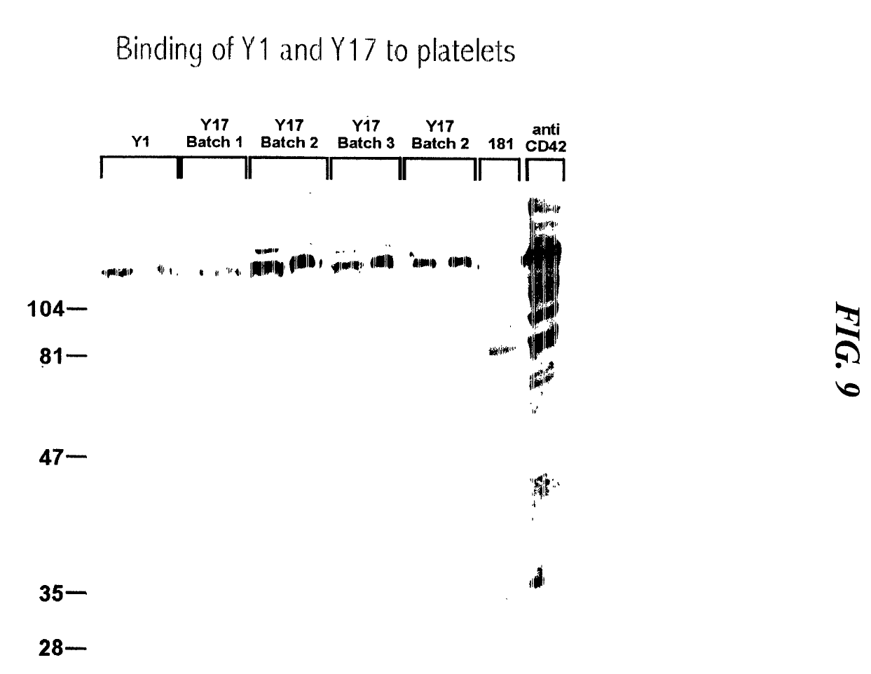

Western Blots show that Y1 and Y17 bind similarly to platelet lysates. (FIG. 9).

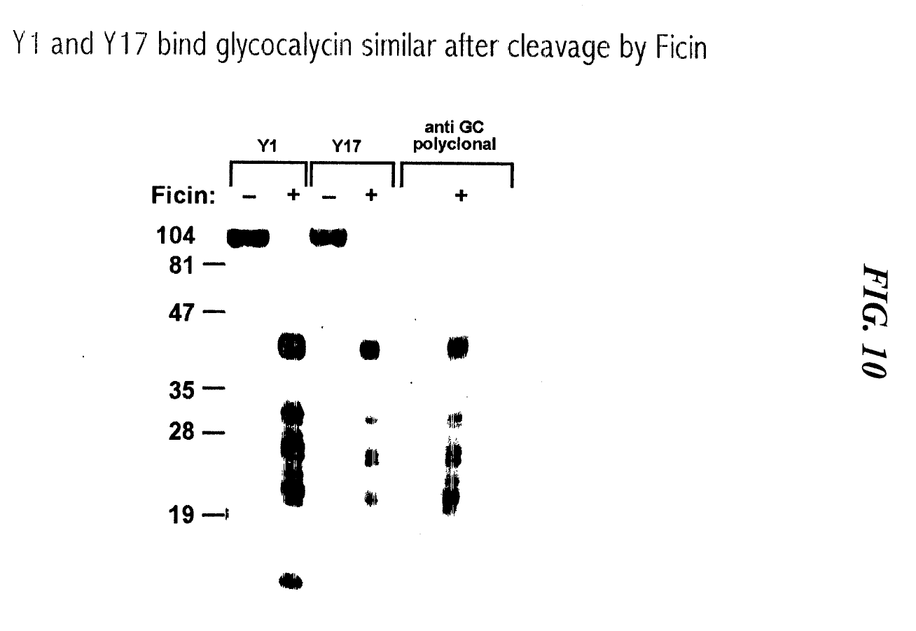

Y1 and Y17 also bind similarly to glycocalicin after cleavage of glycocalicin by O-Sialoglycoprotein Endoprotease or Ficin. (FIGS. 5 and 10).

FACS analysis indicated that Y1 have similar binding profiles to platelets and KG-1. In addition, both do not bind to Raji and T2 cells. In contrast, TM1 (SEQ ID NO: 209), Y16 (SEQ ID NO: 210)and Y45 do not bind to any of the above mentioned human cell lines.

These results demonstrate that Y1 and Y17, two monoclonal antibody fragments of the present invention, share an epitope on various cells, and that this epitope is not recognized by any other tested monoclonal antibodies.

Cathepsin G Cleavage of GPIb—Mapping of the Y1 Epitope

Cathepsin G (Sigma C4428), a neutrophil serine protease, cleaves glycocalicin at a first cleavage site between residues Leu-275 and Tyr-276 and at a second cleavage site between residues Val-296 and Lys-297. Cathepsin G treatment of glycocalicin generates two N-terminal fragments: a small N-terminal 42 kDa fragment (His1-Leu275), a large N-terminal 45 kDa N-terminal fragment (His 1-Val-296), and corresponding ˜95 kDa C-terminal fragments. (FIG. 1).

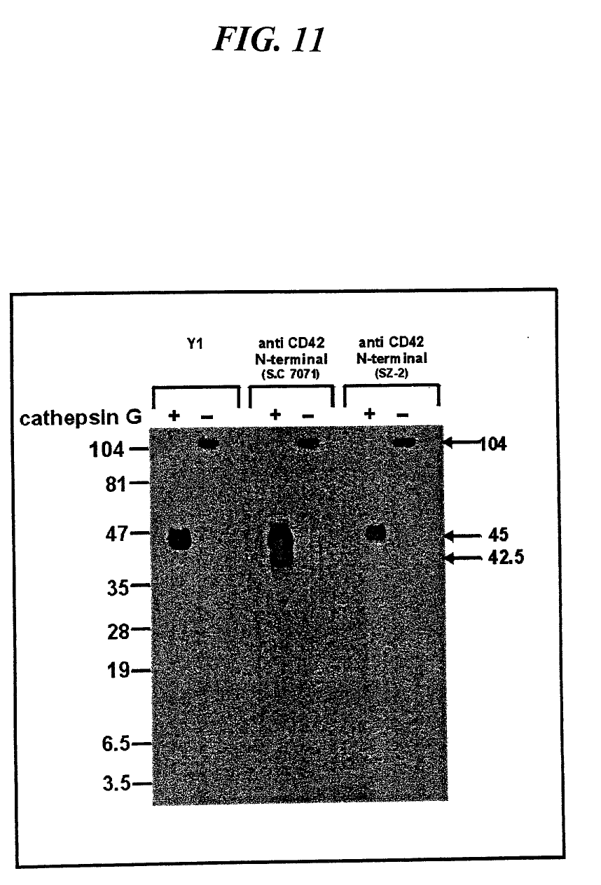

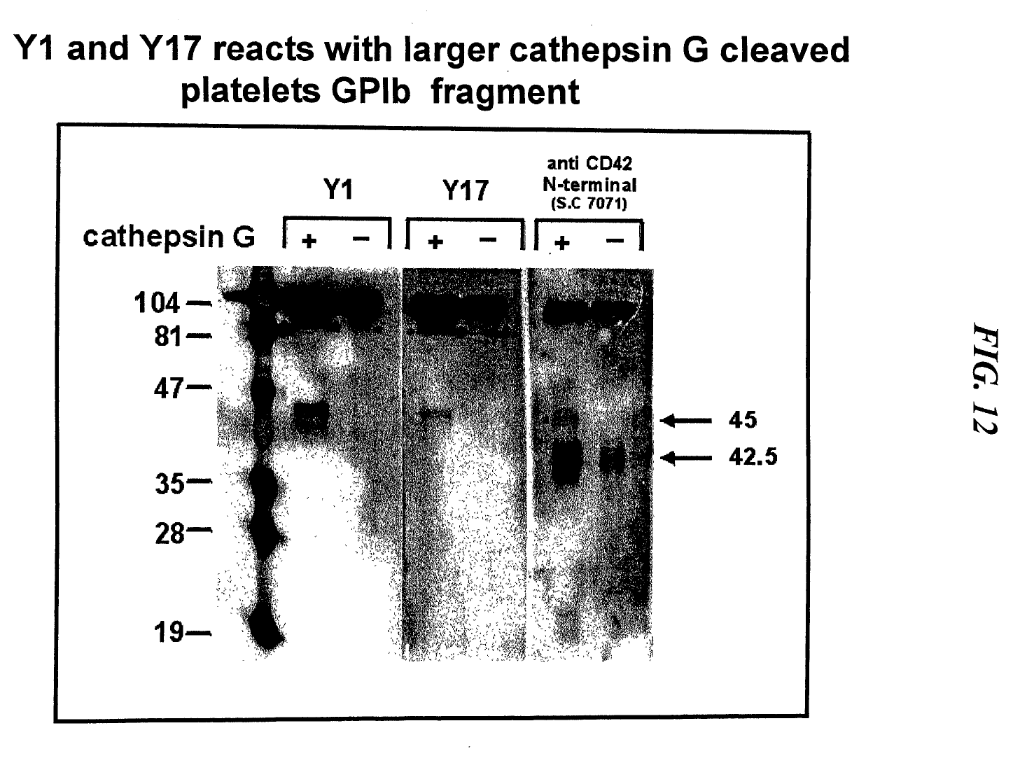

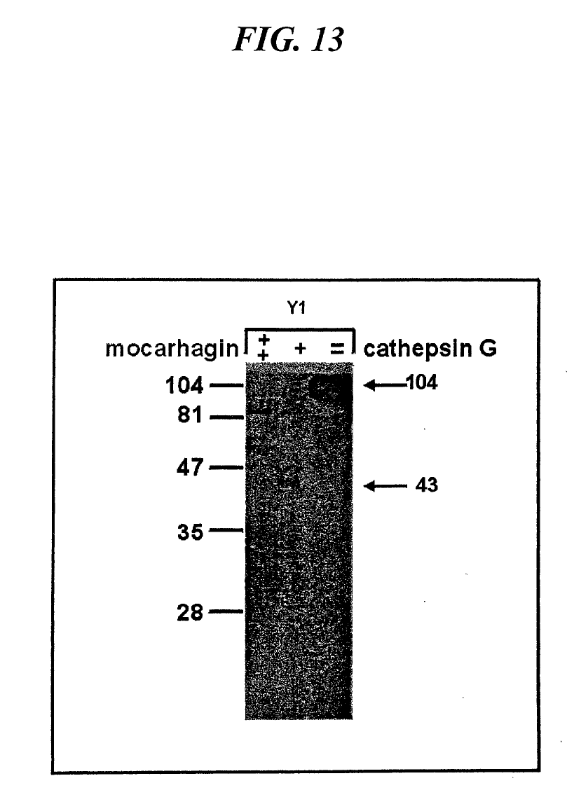

Glycocalicin and glycocalicin fragments generated by cathepsin G digestion were electrophoresed on SDS-polyacrylamide gels and transferred to nitrocellulose for Western analysis. In immunoblots, Y1 bound to the larger N-terminal fragment (His 1-Val-296), but not to the smaller N-terminal fragment (His 1-Leu275), nor to the C-terminal fragment. Likewise, commercial monoclonal antibody SZ2 (Immunotech 0719), which is known to recognize an epitope on GPIbα between residues Tyr276 and Glu282 also reacts only with the larger N-terminal fragment. (FIGS. 11 and 12).

Moreover, monoclonal antibody S.C.7071 which is known to recognize an epitope between His 1 and Leu 275, bound to both N-terminal fragments. Y1 does not bind to the His 1-Leu 275 fragment bound by S.C.7071. These results suggest that the epitope for Y1 is localized between the first and second cathepsin-G cleavage site that is within the sequence Tyr 276-Val 296 or more probably between amino acids ˜276 to 282.

Effect of Synthetic Partial GPIbα Peptides on Y1 Binding to Purified Glycocalicin and to Washed Platelets (WP)

ELISA assays were developed to evaluate the effect of the GPIb derived synthetic peptides on the binding of Y1 to purified glycocalicin. In addition, FACS analysis using washed platelets was carried out. To evaluate the importance of sulfated tyrosine within the Y1 binding site of GPIb, a competitive binding FACS analysis was used. Y1-scFv at a concentration of 1 μg was preincubated with different peptides at concentrations of 2.5 and 200 μM. After a preincubation for 30 minutes at room temperature the mixture was added to a tube containing ˜10⁷ washed platelets and the binding of Y1 to the washed platelets was assessed using polyclonal rabbit anti-scFv-PE. The inhibitory effect of the peptides compared to control binding (Y1 alone) was evaluated by measuring the residual binding of Y1 to washed platelets. The peptides and the results are described in Table 1 and are similar to results that were observed using the same peptides in an ELISA assay (Table B). In both assays, a control level of Y1 binding was determined, as follows. A polystyrene microtiter maxisorb plate was coated with (a) purified glycocalicin or (b) washed platelets. After extensive washing, 0.5 μg/well of Y1 was added. The plate was then incubated with rabbit anti-scFv followed by addition of anti rabbit-HRP (horse radish peroxidase) and HRP substrate. The level of anti rabbit-HRP binding was measured by the intensity of the color produced, and the level of anti rabbit-HRP binding correlates with the level of binding of anti Y1-scFv and the level of binding of Y1. The optical density was measured at A₄₀₅. Each sample was assayed in duplicate, and the average was calculated.

The effect of synthetic GPIbα peptides on Y1 binding to purified glycocalicin was evaluated by mixing varying concentrations of the peptides with a constant amount of Y1. After a preincubation for 30 minutes at room temperature, the mixture was added to a polystyrene microtiter maxisorb plate coated with purified glycocalicin, as described for evaluation of Y1 binding in the absence of peptides. The inhibitory effect of the peptides was evaluated by measuring the residual binding of Y1to glycocalicin using rabbit anti Y1scFv and anti rabbit -HRP antibodies, as described for evaluation of Y1 binding in the absence of peptides. This study was performed with four peptides representing various subsets of the sequence 268 to 285 and a control peptide. Each peptide was tested at different concentrations: 200 μM, 25 μM, 2.5 μM, and 0.5 μM.

The five peptides are as follows in Table 1:

Table 1

| | | |
|---|---|---|
| Peptide Name | Characterization | Sequence |

| | | |
|---|---|---|
| EGR | negative control peptide | REEGRQHFFLLEGRSSYS |
| P-1 | residues 268-285 of GPIbα | GDEGDTDLYDYYPEEDTE |
| P-1-S | residues 268-285 of GPIbα | GDEGDTDLY*DY*Y*PEEDTE |
| P-2-S | residues 273-285 of GPIbα | TDLY*DY*Y*PEEDTE |
| P-3-S | residues 268-280 of GPIbα | GDEGDTDLY*DY*Y*P |

Y* is identical to Y which is sulfated tyrosine.

The results obtained from these assays are presented in Tables 2 and 3 below.

TABLE 2
Effect of Synthetic GPIbα Peptides on Y1 Binding to Glycocalicin
0.25 μg/well Y1

Residual Binding of Y1 (% of baseline)

| Peptide | | | | |
|---|---|---|---|---|
| Concentration | 200 μM | 25 μM | 2.5 μM | 0.5 μM |
| EGR | 85 | 89 | 100 | 121 |
| P-1 | 61 | 71 | 94 | 88 |
| P-1-S | 0 | 25 | 62 | 89 |
| P-2-S | | 15 | 52 | 78 |
| P-3-S | | 21 | 67 | 80 |

These results clearly show that the inhibitory effect of the peptides containing sulfated tyrosine is significantly higher than that observed for the non-sulfated peptide. This effect is dose-dependent, and peptides containing longer N′ (upstream) flanking sequences had a higher inhibitory effect than peptides with extended C′ (downstream) flanking sequences. These results clearly support the conclusion that sulfated tyrosine is required for Y1 binding to GPIbα, and that sequences upstream and downstream from the sulfated region enhance Y1 binding to GPIbα.

TABLE 3
Effect of Synthetic GPIbα Peptides on Y1 Binding to Washed
Platelets as Described By Comparative FACS Analysis

| Peptide | Residual Binding of Y1 (% of baseline) (Geo Mean) | |
|---|---|---|
| Concentration | 200 μM | 2.5 μM |
| EGR | 119 | 96 |
| P-1 | 87 | 106 |
| P-1-S | 5 | 41 |
| P-2-S | 7 | 61 |
| P-3-S | 26 | 82 |
| Control - No | 114 | |
| Peptide | | |

These results further support the hypothesis that sulfated tyrosine residues within the specific region are important for Y1 recognition on GPIb. Overall, analysis of N-terminal peptide proteolytic fragments of mocarhagin and cathepsin G suggest that the GPIbα amino acid sequence Tyr276-Glu-282 is or contains an important epitope for binding of Y1. (FIGS. Tab 1C pages 6 and 7). Further characterization indicated that in addition to residues 276-282 (sulfated anionic sequence) of glycocalicin, upstream amino acids 283-285 are involved in the recognition site of Y 1.

Biological Activity of Y1 scFv, Y17 scFv and IgG Y1 on Platelets Function

Localization experiments suggested that the Y1 binding site resides at the alpha-thrombin and vWF binding sites, which are important for platelet aggregation. Therefore the binding of Y1 scFv, Y17 scFv, and Y1 IgG to washed platelets and to platelet-rich-plasma was studied to determine the effects of the binding on platelet aggregation.

Effect of Y1-scFv and Y17-scFv on Agglutination of Washed Platelets (W.P.)

Aggregation is determined in PRP due to the presence of thrombotic agents, while agglutination is determined in washed platelets. The effect of Y1 (scFv) on agglutination of washed platelets was tested at various concentrations of Y1. Platelets were pre-incubated with Y1 scFv, Y17 scFv, Y16-scFv, or a control TM-1 scFv for 4 min at 37° C. before being exposed to ristocetin, an inducer of platelet agglutination and aggregation.

The results of this study are presented in Table 4 and in FIG. 15. Preincubation of platelets with 25 μg/ml Y1 scFv inhibited agglutination of washed platelets induced by ristocetin. At a Y1 concentration of 12.5 μg/ml, only partial inhibition of platelet agglutination was observed. No inhibition of platelet agglutination was observed at a concentration of 4 μg/ml of Y1. These results indicate that inhibitory activity of Y1 on washed platelet agglutination is dose dependent. Incubation of washed platelets with negative control scFv TM1 had no effect on platelet agglutination induced by ristocetin. Neither Y17 nor Y16, which is another scFv clone selected from the same phage display library and using the same multistep procedure used to select Y1, significantly inhibit agglutination of washed platelets.

TABLE 4

| ScFv Concentration | % inhibition | % agglutination |
|---|---|---|
| TM-1 scFv 25 μg/ml | 10 | 90 |
| Y1 scFv 25 μg/ml | 77 | 23 |
| Y1 scFv 12.5 μg/ml | 33 | 67 |
| Y1 scFv 4 μg/ml | 8 | 92 |
| Y17 scFv 25 μg/ml | 15 | 85 |
| Y16 scFv 38 μg/ml | 14 | 86 |

*100% agglutination is calibrated on the basis of ristocetin treatment.

Effect of Y1-scFv and Y17-scFv on Aggregation of Platelet-Rich-Plasma (PRP)

The effect of Y1 (scFv) on aggregation of platelet-rich-plasma (PRP) was tested at various concentrations of Y1. PRP was pre-incubated with Y1 scFv, Y17 scFv, or a control sTM-1cFv for 4 min at 37° C. before being exposed to ristocetin, an inducer of platelet agglutination and aggregation. A reversible inhibitory effect was observed when scFv was added to PRP prior to the addition of ristocetin, and it was dose dependent.

The results of this study are presented in Table 5 and in FIG. 16. Y1 at a final concentration of 50 μg/ml inhibited ˜80% of platelet aggregation in platelet rich plasma induced by ristocetin as was recorded during the first 4 minutes. There was no significant inhibition of platelet aggregation at a Y1 concentration of 25 μg/ml. Y17 did not inhibit aggregation of platelets. Incubation of washed platelets with 50 μg/ml of the negative control scFv, TM 1, had no effect on platelet aggregation induced by ristocetin. (Table 5).

A comparison between washed platelets and PRP indicated that (1) scFv Y1 has an inhibitory effect on platelet aggregation and agglutination induced by ristocetin; (2) the effect is dose dependent; (3) higher inhibitory effect is observed in washed platelets relative to PRP; (4) reversible inhibitory effect was detected in PRP; (5) neither TM1 not Y16 scFv antibody fragments has an effect; and (6) Y17 is a negative control in this assay.

TABLE 5

| ScFv Concentration | % inhibition | % aggregation |
|---|---|---|
| TM-1 scFv 50 μg/ml | 0 | 100 |
| Y1 scFv 50 μg/ml | 80 | 20 |
| Y1 scFv 25 μg/ml | 13 | 87 |
| Y17 scFv 38 μg/ml | 0 | 100 |

*100% agglutination is calibrated on the basis of ristocetin treatment.

Effect of Y1-IgG on Agglutination of Washed Platelets (W.P.)

Due to its natural structure the full IgG Y1 has two binding sites on GPIbα and one binding site for an Fc receptor. It is likely that if full IgG Y1 binds two GPIbα molecules, it will activate platelets and induce platelet agglutination. Furthermore, because platelets have an Fc-receptor, Y1-IgG can induce platelet agglutination by binding to GPIbα and to an Fc-receptor, thereby producing platelet agglutination by each IgG Y1 binding to three platelets. Therefore, the effect of IgG Y1 on aggregation of washed platelets was tested at different concentrations of Y1-IgG in the presence or absence of ristocetin. Induction of platelet aggregation by Y1-IgG was monitored for 4 min at 37° C., followed by addition of ristocetin.

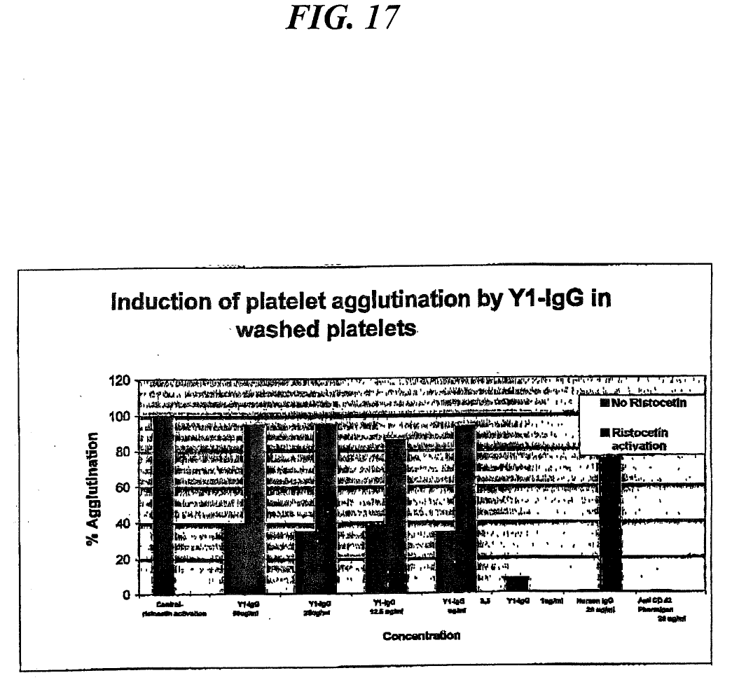

The results are presented in Table 6 and FIG. 17 without agonist. Y1-IgG alone at a final concentration of 50 μg/ml induced platelet agglutination ˜39% of normal agglutination of washed platelets. Induction of platelet agglutination by Y1-IgG was tested for 4 min at 37° C., followed by addition of ristocetin. No additional effect on platelet agglutination was seen after the addition of ristocetin: normal platelet agglutination was observed. However, there was no induction of platelet agglutination when platelets were incubated with 1 μg/ml Y1.

There was no reduction of platelet agglutination when a commercial monoclonal antibody against GPIbα (CD42) (Pharmigen), which inhibits platelet agglutination, or control human IgG-Lambda (Sigma) were used as above.

TABLE 6

| | | | % inhibition | | % agglutination | |
|---|---|---|---|---|---|---|
| IgG Ab | | | Without | With | Without | With |
| Concentration | | | Ristocetin | ristocetin | ristocetin | ristocetin |
| Y1-IgG | 50 | μg/ml | 61 | 5 | 39 | 95 |
| Y1-IgG | 25 | μg/ml | 65 | 5 | 35 | 95 |
| Y1-IgG | 12.5 | μg/ml | 62 | 5 | 38 | 95 |
| Y1-IgG | 3.5 | μg/ml | 66 | 14 | 34 | 86 |
| Y1-IgG | 1 | μg/ml | 92 | 7 | 8 | 93 |

| | | | | |
|---|---|---|---|---|
| Mouse anti-human | 99.5 | 100 | 0.5 | 0 |

| | | |
|---|---|---|
| CD42 | 20 | μg/ml |
| IgG | | |

| | | | | |
|---|---|---|---|---|
| Control human | 99.5 | 25 | 0.5 | 75 |

| | | |
|---|---|---|
| IgG | 20 | μg/m |

| | | | | |
|---|---|---|---|---|
| Control ristocetin | — | 0 | — | 100 |
| Activation | | | | |

Effect of Y1-IgG on Aggregation of Platelet-Rich-Plasma (PRP)

The effect of Y1-IgG on aggregation of Platelet-Rich-Plasma was tested at different concentrations of Y1-IgG in the presence or absence of ristocetin. Induction of platelet aggregation by Y1-IgG was tested for 4 min at 37° C., followed by addition of ristocetin.

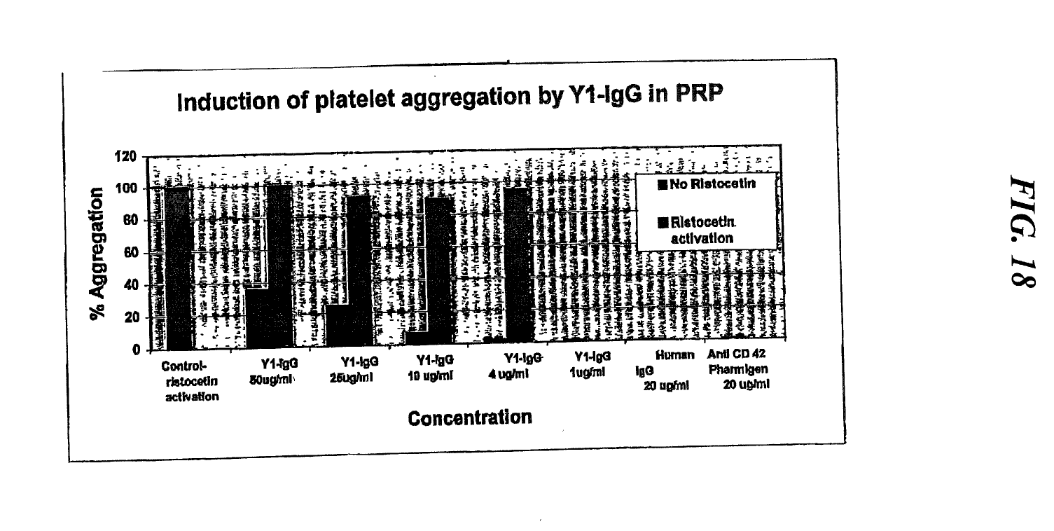

The results are presented in Table 7 and FIG. 18. No effect on platelet aggregation was seen after the addition of ristocetin: normal platelet aggregation was observed. Y1-IgG at a final concentration of 50 μg/ml induced platelet aggregation in Platelet-Rich-Plasma, before the addition of ristocetin. Y1-IgG at a concentration of 25 μg/ml only partially induced platelet aggregation before the addition of ristocetin. No induction of platelet aggregation was observed with Y1-IgG concentrations of 10 μg/ml, 4 μg/ml, or 1 μg/ml. Commercial monoclonal antibodies against GPIbα (Pharmigen), which inhibit platelet aggregation at concentration of 20 μg/ml, did not induce platelet aggregation. Control human IgG-Lambda (Sigma) in the same concentration as Y1-IgG also did not induce platelet aggregation.

TABLE 7

| | |
|---|---|
| % inhibition | % agglutination |

| IgG | | | Without | With | Without | With |
|---|---|---|---|---|---|---|
| Concentration | | | Ristocetin | ristocetin | ristocetin | ristocetin |
| Y1-IgG | 50 | μg/ml | 64 | 0 | 36 | 100 |
| Y1-IgG | 25 | μg/ml | 75 | 8 | 25 | 92 |
| Y1-IgG | 10 | μg/ml | 93 | 10 | 7 | 90 |
| Y1-IgG | 4 | μg/ml | 98 | 5 | 2 | 95 |
| Y1-IgG | 1 | μg/ml | 95.5 | 0.5 | 0.5 | 99.5 |

| | | | | |
|---|---|---|---|---|
| Human anti-CD42 | 99.5 | 0.5 | 0.5 | 99.5 |

| | | |
|---|---|---|
| IgG | 20 | μg/ml |

| | | | | |
|---|---|---|---|---|
| Control ristocetin | — | 0 | — | 100 |
| Activation | | | | |

Identification of Y1 Plasma Soluble Ligands and Cell Lines

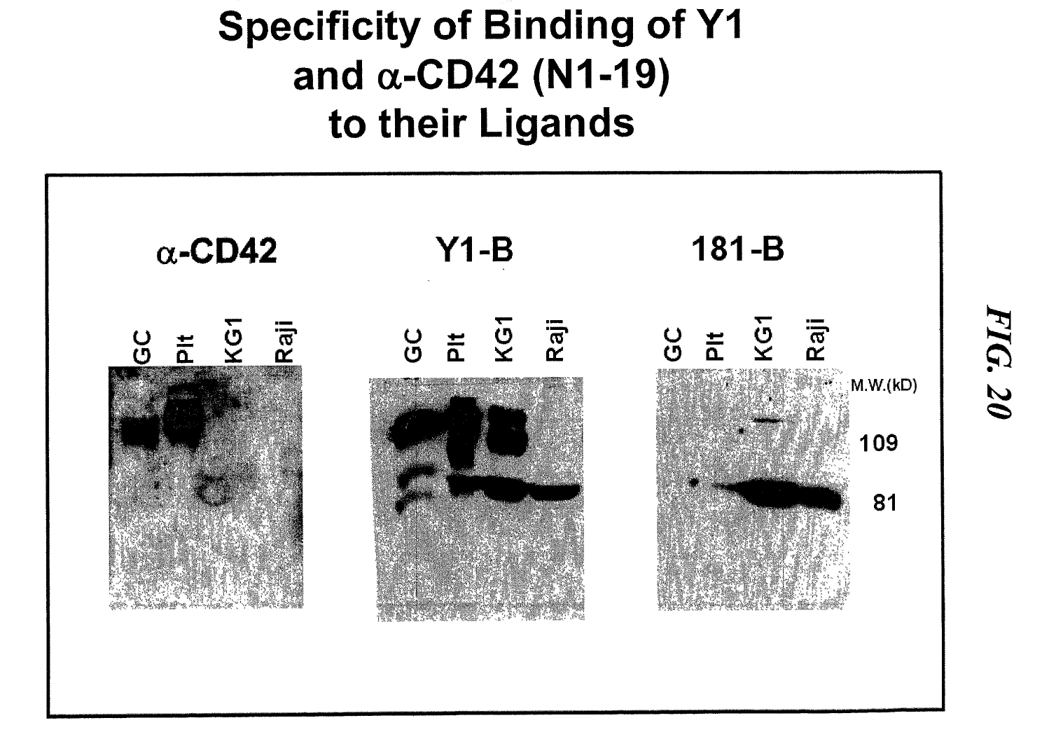

Antibodies against GPIbα (CD42b) recognize platelet lysate and glycocalicin and but not KG-1 cell lysate (a Y1 binding positive myeloid cell line) or Raji cell lysate (a B cell line that is negative for Y1 binding at concentrations at which KG-1 cells are positive for Y1 binding). In contrast, Y1 recognized both glycocalicin, platelet lysate, and KG-1 cells, but not Raji cell extract. The negative control scFv-181, did not recognize any of the relevant proteins. (FIG. 20).

The uniqueness of Y1 cross-reactivity was further demonstrated in a comparative analysis between Y1 and SZ2 (Mab against the sulfated region of GPIb). In contrast to SZ2, Y1 binds not only to GPIb, but also to plasma proteins and to myeloid derived cell extracts as described below.

Y1 Ligands in Human Plasma

Two proteins immunoreacted with Y1 both in normal as well as in leukemia patients plasma. The first is designated H P-ligand 1, which has a molecular mass of ˜50 kDa under reducing conditions and >300 kDa under non-reducing conditions and which completely disappears from the serum after coagulation; and (2) H P-ligand 2, which has a molecular mass of ˜80 kDa under both reducing and non-reducing conditions and which remains in serum after coagulation. After purification using a Q-Sepharose column reverse phase (RP-HPLC) 2D gel electrophoresis, and peptide mapping, the ˜50 kDa ligand was identified as the normal variant of the gamma chain (y prime) of human fibrinogen. The sequence VRPEHPAETEYDSLYPEDDL, is present only in fibrinogen gamma prime, but not the abundant form of fibrinogen gamma, and is similar to GPIb anionic region containing sulfated tyrosine. Most likely this is the binding site for Y1. The ˜80 kDa was identified as complement compound 4 (CC4) and Lumican. As above, it contains sulfated tyrosine residues accompanied by a stretch of negatively charged amino acids.

Binding of Y1 to Primary Leukemia Cells

FACS analysis indicates that Y1 binds selectively to leukemia cells, but not to normal blood cells both in normal blood sample and normal cells within the blood of leukemia samples. A summary of the results from patient analysis is shown in the following tables.

TABLE 8
Results of the patients with Y1

| Disease | Number Positive | % Positive |
|---|---|---|
| Multiple Myeloma | 16/16 | 100% |
| AML | 60/75 | 80% |
| B-Leukemia | 29/43 | 67% |

TABLE 9
B-Leukemia

| Type | Source | Number Positive | % Positive | % Negative |
|---|---|---|---|---|
| Pre-B-ALL | BM | 3/3 | 100 | 0 |
| B-ALL | BM | 3/9 | 33 | 67 |
| B-CLL | PB | 17/23 | 74 | 26 |
| B-Lymphoma | PB | 5/8 | 62 | 38 |

BM = Bone Marrow
PB = Peripheral Blood

Characterization of Y1 Epitope on Myeloid Cells (KG-1)

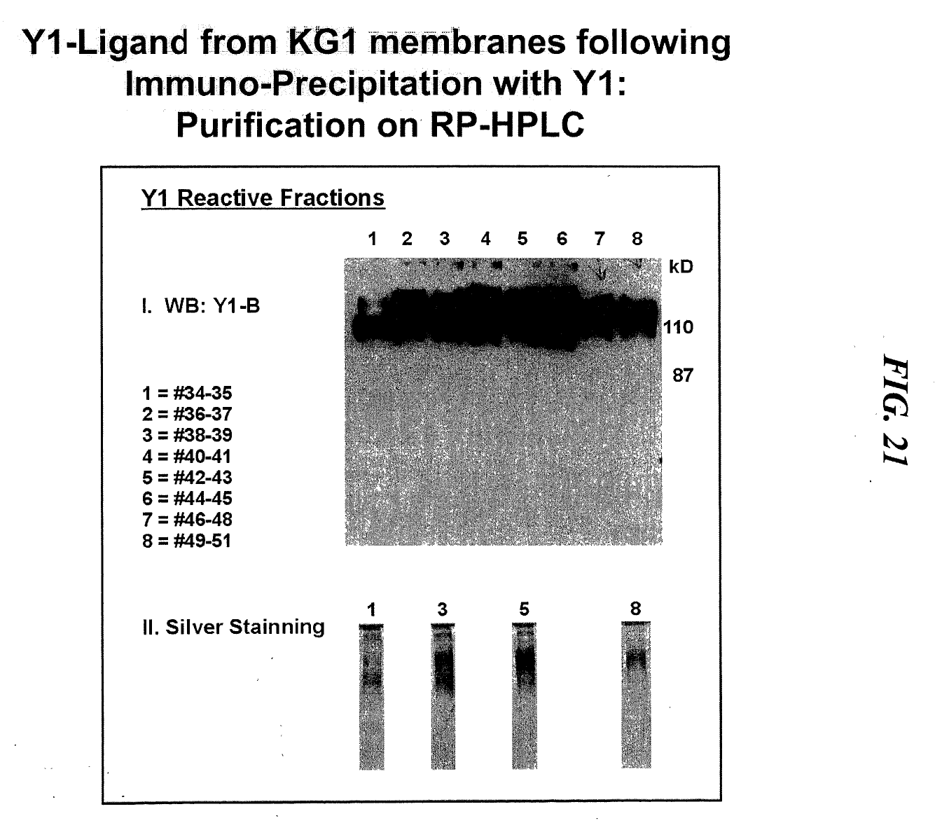

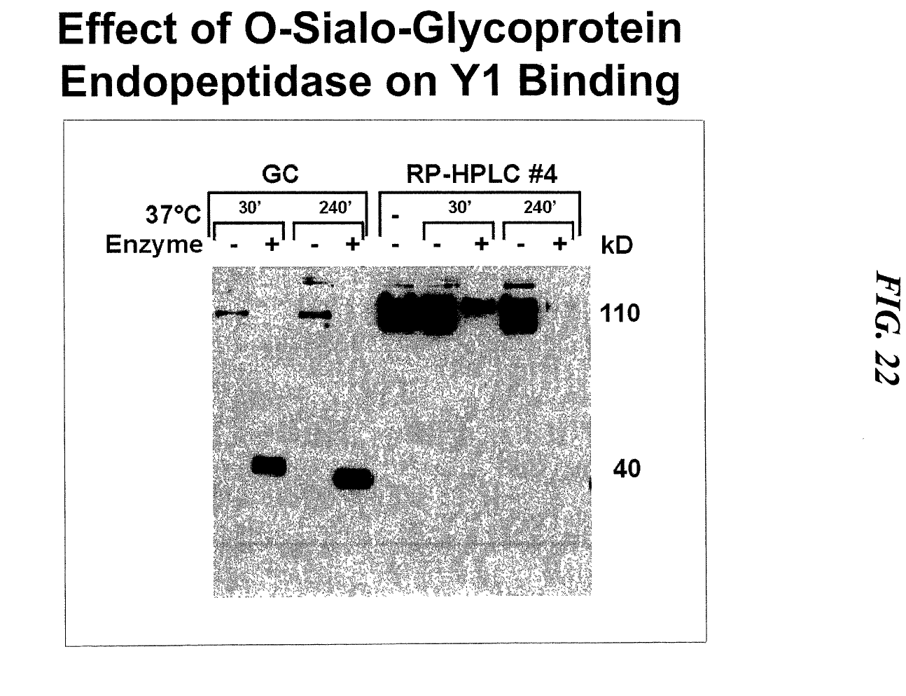

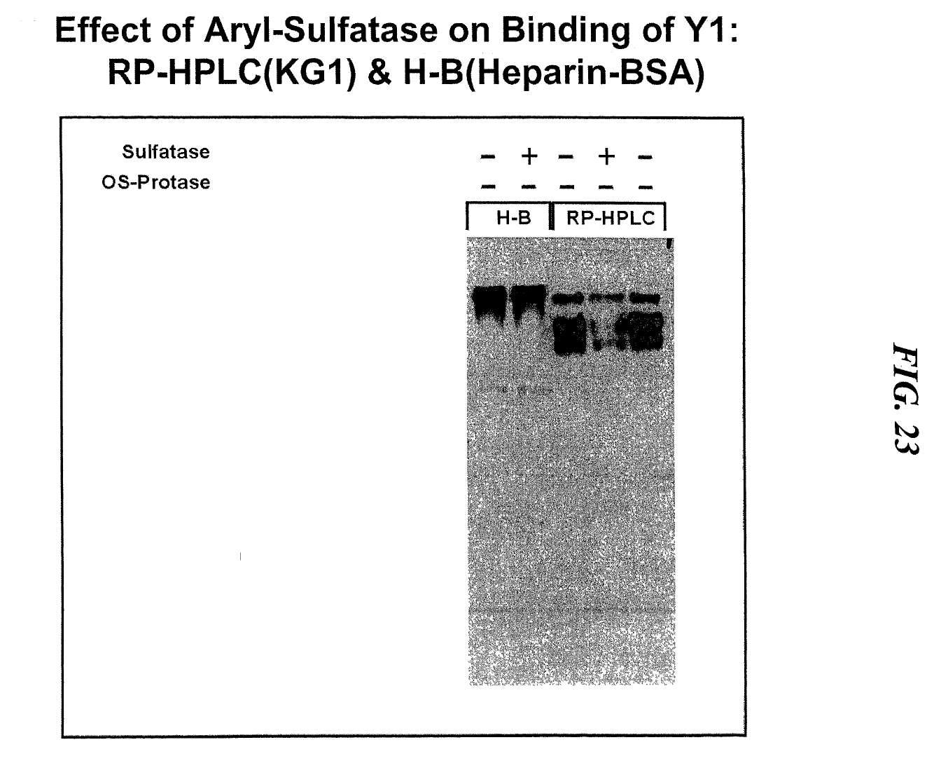

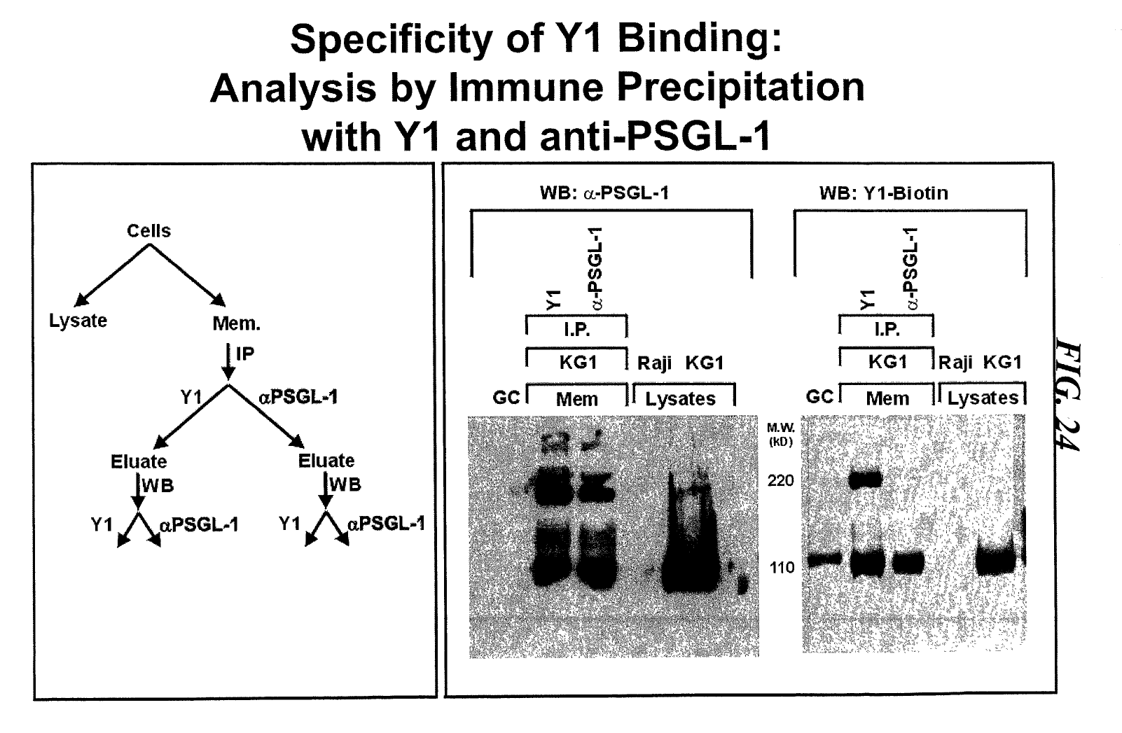

Approximately 25 billion KG-1 cells were collected for the purification of the Y1 epitope from the KG-1 cell membranes. KG-1 membrane preparations were found to contain at least 2 subunits to which Y1 binds: a ˜110 kDa subunit and a ˜120 kDa subunit. Y1 also binds to a ˜220 kDa subunit, which may be a dimer of the ˜110 kDa subunit. Purification of Y1 epitope was accomplished by immunoprecipitation with Y1, and reverse phase (RP-HPLC). 2 μl of the pooled fractions were used for Western blotting with scFv Y1, and 40 μl were used for silver staining. (FIG. 21).

Y1 ligand was further characterized using enzymatic treatments with proteases, glycanases, and sulfates; Western blotting with Y1, anti-CD42 antibodies, anti-CD162 antibodies and 181, immunoprecipitation using Y1 and anti-CD162 antibodies; FACS analysis using Y1 anti-CD162 antibodies; and sequencing.

The table below summarizes the biochemical experiments preformed to characterize and localize the Y1 binding site on KG-1 cells.

TABLE 10
Western Blot Analysis with Y1 on SDS-PAGE Reducing Gels

| | | | Reactivity | Presented |
|---|---|---|---|---|
| | | | with | in |
| Substrate | Treatment | Condition | Y1 | FIG. |
| RP-HPLC KG-1 | O-Sialo | 30′ at | Reactivity | Tab 2A |
| membrane | glycoprotein | 37° C. | only with | slide 14 |
| fraction | endopeptidase | | the | |
| | | | 120 kDa | |
| | | | form | |
| RP-HPLC KG-1 | O-Sialo | 4 hr at | No | Tab 2A |
| membrane | glycoprotein | 37° C. | reactivity | slide 14 |
| fraction | endopeptidase | | | |
| RP-HPLC KG-1 | aryl-sulfatase | 18 hr at | No | Tab 2A |
| membrane | | 22° C. | reactivity | slide 14 |
| fraction | | | | |
| RP-HPLC KG-1 | mocarhagin | 7′ at | No | Tab 2A |
| membrane | | 37° C. | reactivity | slide 14 |
| fraction | | | | |
| Glycocalicin | O-Sialo | 30′ at | Enhanced | Tab 2A |
| (GC) | glycoprotein | 37° C. | binding | slide 14 |
| | endopeptidase | | | |
| Heparin - BSA | aryl-sulfatase | 18 hr at | Binds to | Tab 2A |
| | | 22° C. | Y1 as | slide 16 |
| | | | without | |
| | | | treatment | |

In summary, following treatment with endopeptidases the Y1 signal is cleaved off and cannot be detected. Most likely, the fragment containing the Y1 binding site is found on the N′-terminus and it is too small to be determined under the conditions used in the above experiments. In addition, the results obtained with the aryl-sulfatase which remove sulfate entities from proteins (within the KG-1 cell extract), but not from sugar moieties (on the heparin) further support our hypothesis that sulfate is required for Y1 recognition. Interestingly, O-Sialo glycoprotein endopeptidase enhanced the Y1 signal in the GC cleavage product. We assume that following this treatment the Y1 binding site, now located at the C′ terminus is better exposed to the Y1 binding.

Correlation Between Y1 and PSGL-1 Antibody-KPL1: Western Blot Analysis

The binding of scFv Y1 antibody and commercially available anti-PSGL-1 monoclonal antibody (KPL1) to KPL1 immunoprecipitated (IP) membrane proteins derived from KG-1 cells was assessed. A Raji cell lysate was used as a Y1 and KPL1 negative control.

The membrane fraction of KG-1 cells was immunoprecipitated with KPL1. The IP fraction was further immunoprecipitated either with scFv Y1 antibody or with KPL1. The non-precipitated (eluate) fractions were analyzed by Western blot, using either scFv Y1 or KPL1 antibodies.

Both the immunoprecipitation scheme and the results are shown in FIG. 24. KPL1 does not recognize glycocalicin. However, both scFv Y1 and KPL1 antibodies recognize membrane proteins on KG-1 cells.

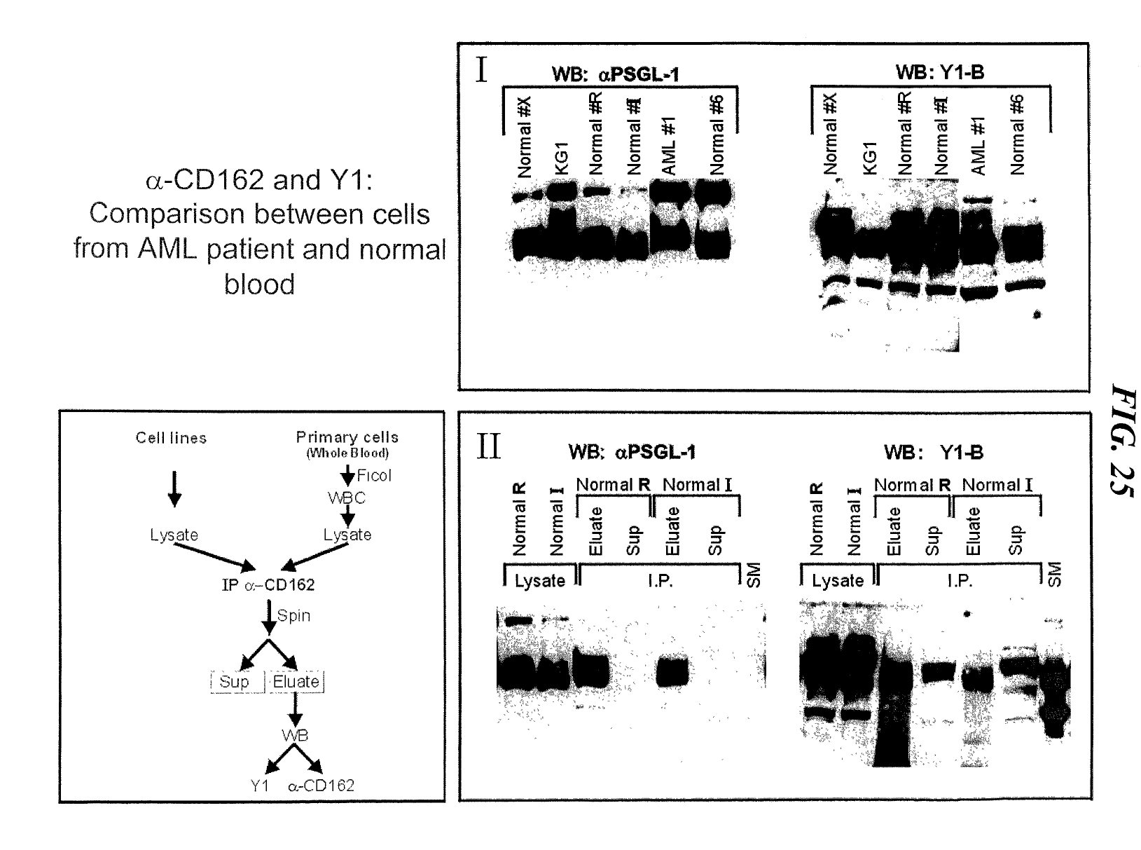

Lysates from cell lines and primary white blood cells were immunoprecipitated with anti-CD162 antibodies and centrifuged to produce a supernatant and an eluate. Western blot analysis of the proteins present in the eluate and supernatant was performed using scFv Y1 and anti-CD162 antibodies. KG-1 membrane preparations contain two subunits (˜110 kDa and ˜120 kDa) to which anti-CD162 (PSGL-1) antibodies bind. In contrast, normal white blood cell membrane preparations have only the smaller subunit. Membrane preparations from AML patients have only the larger subunit. scFv Y1 binds to a distinct species, which is found in the supernatant of the immunoprecipitation, and to which anti-CD162 antibodies do not bind. (FIG. 25).

FACS Analysis

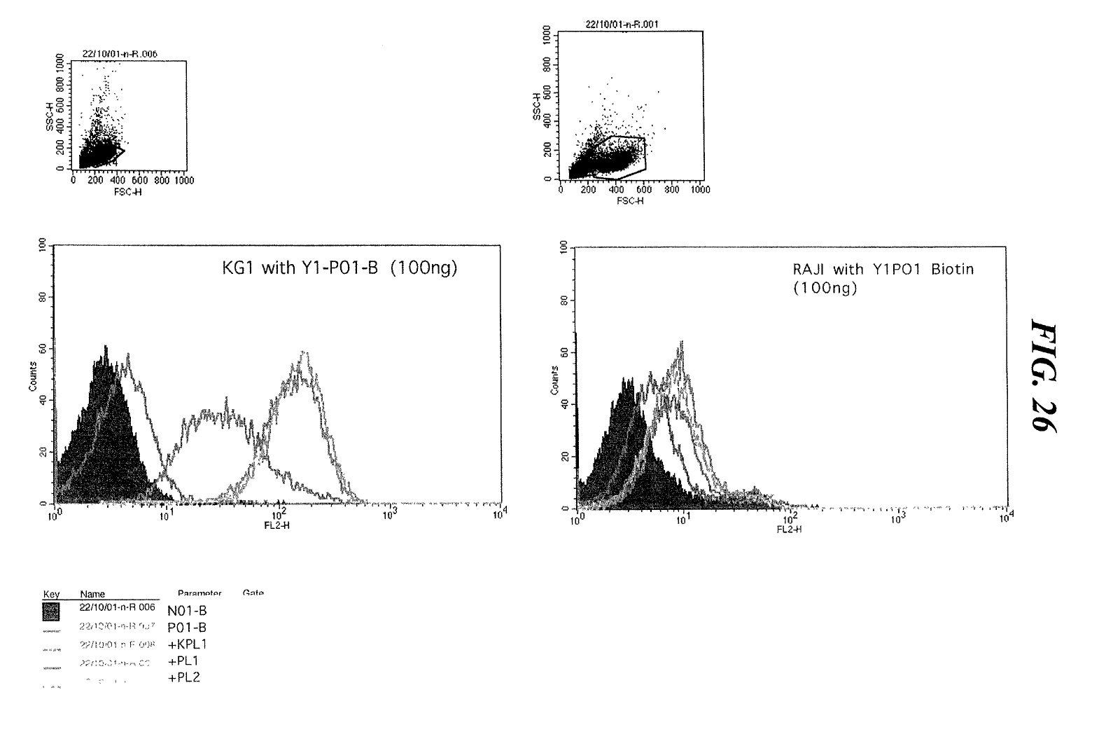

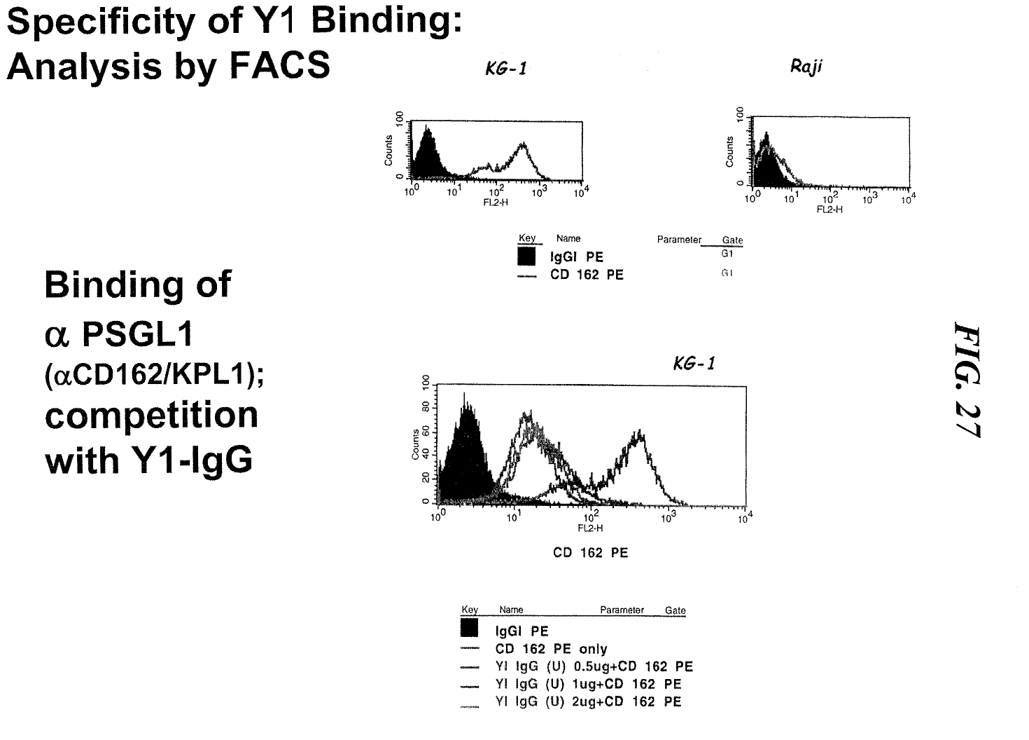

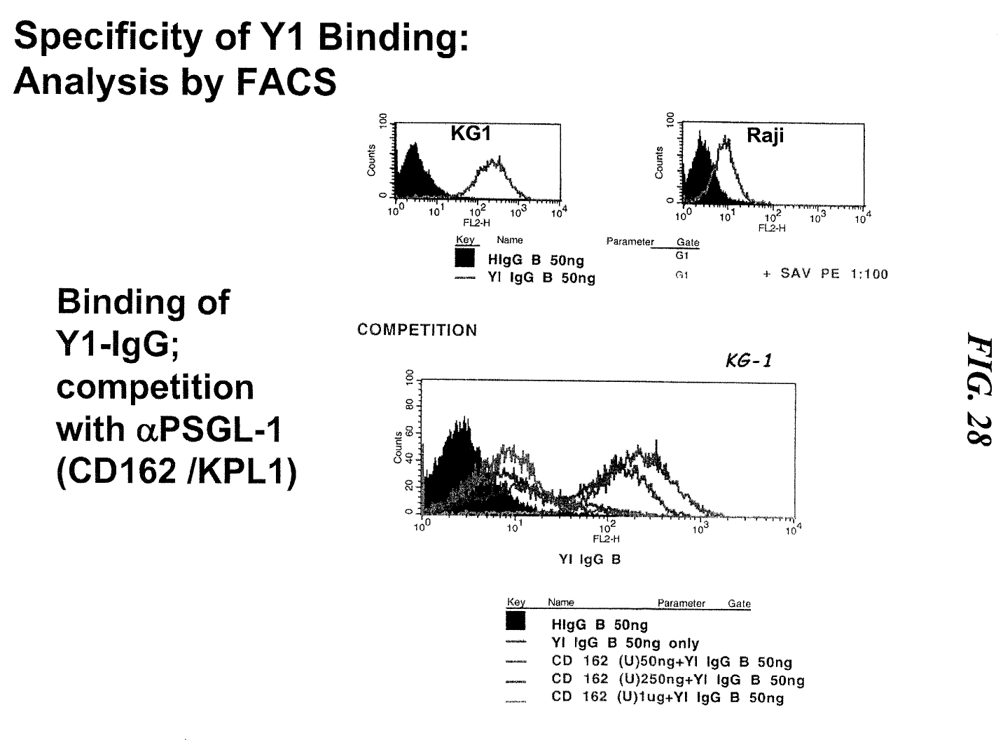

The binding of Y1 antibody (both the scFv and the IgG forms) to KG-1 cells in the presence of anti-PSGL-1 (anti-CD162) (KPL1) antibodies was assessed in competitive binding assays using FACS analysis. To this end, different commercially available anti-PSGL-1 antibodies, KPL1 (an antibody that identifies the sulfated tyrosine N-terminal domain of PSGL-1), PL1 (an antibody that identifies the non-sulfated N-terminal domain of PSGL-1), and PL2 (an antibody that identifies a non-sulfated internal domain of the PSGL-1 receptor) were used. Only KPL1 completely inhibits the binding of Y1 to KG-1 cells, while PL1 partially inhibits binding. There is no inhibition of binding in the presence of the PL2 antibody. (FIG. 26) Raji cells did not bind to KPL1 antibodies. Similarly, complete IgG Y1 at different concentrations inhibits the binding of KPL1 antibody to KG-1 cells in a dose dependent mode (FIG. 27) Likewise, KPL1 antibody inhibits the binding of full IgG Y1 antibody to KG-1 cells in a dose dependent mode. (FIG. 28).

Correlation Between Y1 and KPL1 Binding to Primary Leukemia Cells

Analysis of binding of scFv Y1 antibodies and anti-CD162 antibodies to diseased cells also illustrates that scFv Y1 has binding characteristics different from those of anti-CD162 antibodies. Specifically, FACS analysis of Y1 and anti-CD162 binding to Pre-B-ALL, HCL, AML, B-ALL, B-CLL, unclassified leukemia, B-PLL, and multiple myeloma cells from human patients showed the two antibodies have different binding profiles. (Table F). Y1 binds to the leukemic cells in 10 of 12 samples. In contrast, anti-CD162 bound only 5 out of 12 samples. Out of the 12 samples, 5 were found to bind Y1 but not anti-CD162. Thus, it may be concluded that, in leukemic cells, scFv Y1 binds to a ligand other than that recognized by anti-CD162.

TABLE 11
Leukemia samples—Analysis of Anti-CD162 versus Y1

Reaction with the Leukemia Cells

| Patient # | Disease | ScFv Y1 | Anti CD162 |
|---|---|---|---|
| 42291 | Pre-B-ALL | + | − |
| 42299 | HCL | − | − |
| 42311 | AML | + | + |
| 42321 | B-ALL | − | − |
| 42323 | B-CLL | + | − |
| 42325 | Unclassified | + | − |
| 42332 | B-CLL | + | − |
| 42352 | B-PLL | + | +/− |
| 42330 | AML | + | + |
| 42334 | MM | + | − |
| 42366 | AML | + | + |
| 42370 | AML/ALL | + | +/− |

Overall, sulfated-tyrosine containing Y1-binding domains in GP1bα, Fng-γ prime, and PSGL-1, are DEGDTDLYDYYPEEDTEGD (amino acids 269-287), EHPAETEYDSLYPED (amino acids 411-427), and QATEYELDYDFLPETE (amino acids 1-17), respectively. An additional binding site, with a higher affinity to Y1, is most likely to be expressed on primary leukemia cells. Interestingly, blood samples that are positive both to be scFv Y1 and anti-CD162 were derived from AML patients, while B-cell were negative to anti-CD162.

Binding Analysis of Sulfated Peptides to Y1

A competitive binding ELISA assay was used to assess the importance of the presence and position of sulfated tyrosines to the binding of peptides to Y1.

Glycocalicin was immobilized on a Maxisorb plate. scFv Y1 was preincubated with a peptide of interest for 10 minutes at three different concentrations (1, 10 and 100 μM) in order to observe a dose response. (Table 12). After preincubation, the mixture (Y1+peptide) was added to the plate, and binding of scFv Y1 was assessed using polyclonal rabbit anti-V_{L}, which recognizes the V_{L} chain of scFv Y1, followed by anti-rabbit-HRP. In mixtures in which the peptide bound to scFv Y1, a decrease in the binding of scFv Y1 to glycocalicin compared to control binding was observed. In mixtures in which the peptide did not bind to scFv Y1, no change in the binding of scFv Y1 to glycocalicin compared to control binding was observed.

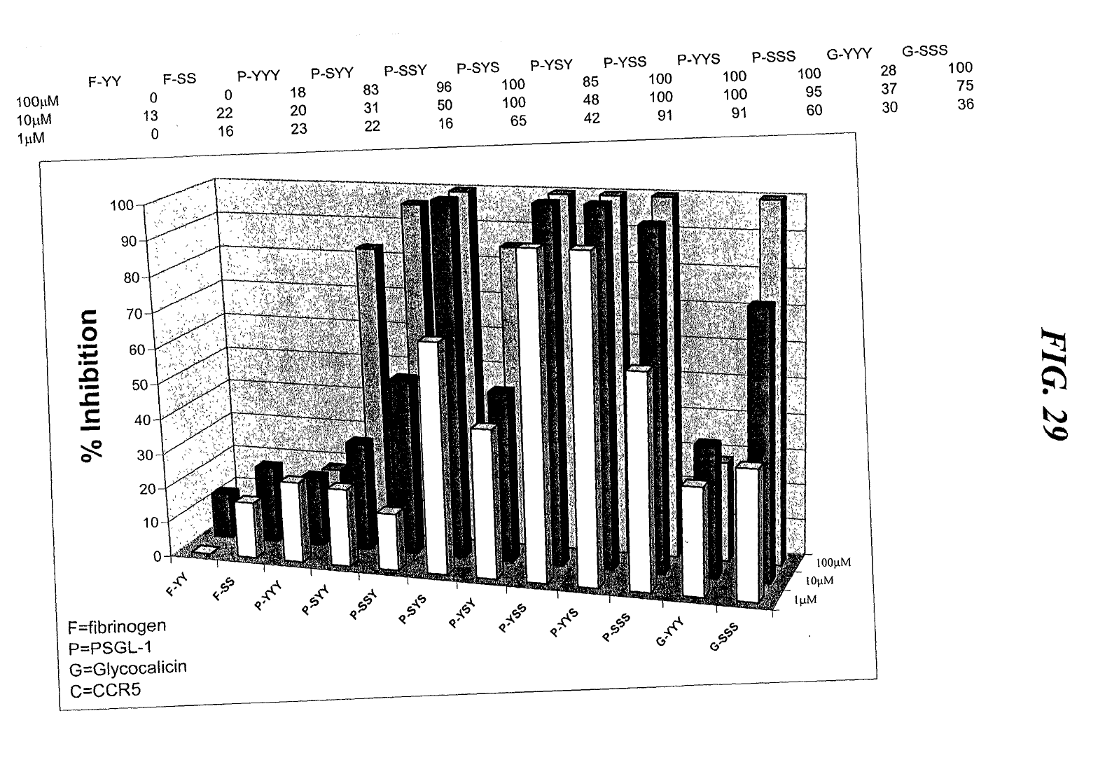

The experiment was done twice, and the results are described in an ELISA graph. (FIG. 29) Peptides derived from fibrinogen did not inhibit the binding of the Y1, regardless of sulfation. Non-sulfated peptides from PSGL-1 did not inhibit Y1 binding to glycocalicin. All sulfated peptides derived from PSGL-1 inhibited Y1 binding to glycocalicin. Peptides P-YYY* and P-YY* Y* were the best inhibitors, followed in efficiency by P-Y*Y Y* then P-YY*Y then P-Y* Y*Y and P-Y*YY. Non-sulfated peptides derived from glycocalicin did not inhibit Y1 binding to glycocalicin, but glycocalicin-derived peptide having the same sequence sulfated on three sulfates (G-Y* Y* Y*) did inhibit the binding, with efficiency similar to that of P-Y Y*Y.

Thus, it is clear that not every sulfated peptide binds to scFv Y1 to the same extent. Also, significantly, these results demonstrate that only one sulfated tyrosine is necessary for binding, as can be seen with peptides P-Y*YY and P-YY Y*. Further, it can be seen that the amino acid context of the sulfated tyrosines influences Y1 binding. For example, P-Y*YY (containing one sulfated tyrosine in the sequence EY*E) inhibits binding efficiently only at 100 μM. In contrast, P-YYY*(containing one sulfated tyrosine in the sequence DY*D) inhibits binding efficiently at 1 μM.

Table 12: Sulfated Peptides

| | Source of | | | | | |
|---|---|---|---|---|---|---|
| Name | Peptide | | Sequence | #aa | MW | Sulfation |
| F-YY | Fibrinogen-γ- | VRPEHPAETEYESLYPEDDL | 20 | 2389 | — | |
| | prime chain | | | | | |
| F-Y*Y* | Fibrinogen-γ- | VRPEHPAETEY*ESLY*PEDDL | 20 | 2549 | Sulfated | |
| | prime chain | | | | | |
| P-YYY | PSGL-1-n- | QATEYEYLDYDFLPETE | 17 | 2126 | — | |
| | terminus | | | | | |
| P-Y*YY | PSGL-1-n- | QATEY*EYLDYDFLPETE | 17 | 2206 | Sulfated | |
| | terminus | | | | | |
| P-Y*Y*Y | PSGL-1-n- | QATEY*EY*LDYDFLPETE | 17 | 2286 | Sulfated | |
| | terminus | | | | | |
| P-Y*YY* | PSGL-1-n- | QATEY*EYLDY*DFLPETE | 17 | 2286 | Sulfated | |
| | terminus | | | | | |
| P-YY*Y | PSGL-1-n- | QATEYEY*LDYDFLPETE | 17 | 2286 | Sulfated | |
| | terminus | | | | | |
| P-YY*Y* | PSGL-1-n- | QATEYEY*LDY*DFLPETE | 17 | 2286 | Sulfated | |
| | terminus | | | | | |
| P-YYY* | PSGL-1-n- | QATEYEYLDY*DFLPETE | 17 | 2286 | Sulfated | |
| | terminus | | | | | |
| G-YYY | GPIbα | GDEGDTDLYDYYPEEDTE | 18 | 2126 | | |
| G-Y*Y*Y* | GPIbα | GDEGDTDLY*DY*Y*PEEDTE | 18 | 2366 | Sulfated | |

Y*=Sulfated Tyrosine

Hypothesis/Conclusions

(1) Y1 resembles L-selectin which recognizes both sulfated protein and sugar moieties, and is distinct from the P-selectin which recognizes only sulfated proteins. Therefore, it can compete for the bonding of both proteins.

(2) Variation in sulfation during differentiation and cell growth may affect Y1 binding. Therefore, Y1 may compete with both P and L selectins for binding to their sulfated ligands.

In vivo Models for Evaluating the Efficacy of the Leukemia-Specific Antibody.

Two human leukemia models were developed in immuno-deficient mice as well as in assay systems.

The human cell lines used were MOLT4 cells derived from a T cell leukemia patient and KG-1 cells derived from an AML patient. Antibodies specific for the relevant human antigens on each cell line were used to identify and quantify malignant cell engraftment.

T-ALL (MOLT4) Model

The in vivo mouse model for T-ALL uses SCID mice (Jackson Laboratories) injected with MOLT4 cells derived from a T cell leukemia patient.

In one experiment, SCID mice were pretreated with 100 mg/kg Cytoxan (CTX, Cyclophosphamid for injection, Mead Johnson). Eleven days after CTX injection, MOLT-4 cells were injected intravenously into the tail vein. Control mice were injected with PBS alone. One week post-MOLT-4 injection mice were injected with CONY1-Doxorubicin, which is a conjugate between scFv CON Y1 polypeptide, having KAK amino acid residues at its carboxy end and doxorubicin via a short organic linker; CONY1, which is a scFv antibody fragment derived from Y1 scFv in which the DNA sequences encoding the myc tag of Y1 were deleted and replaced with a DNA sequence encoding the amino acids lysine, alanine, lysine (KAK); or free Doxorubicin. The mice were injected three times per week for three weeks. Control mice were injected with PBS; and another control group did not receive any treatment. (Table M).

TABLE 13

| Number of Mice | Inoculation | Treatment |
|---|---|---|
| 5 | PBS only | — |
| 9 | MOLT-4 | — |
| 9 | MOLT-4 | CONY-Dox (2.5 mg/kg) |
| 9 | MOLT-4 | CONY-Dox (2.5 mg/kg) |
| 8 | MOLT-4 | Free Dox (0.1 mg/kg) |

Mice started to die 32 days post cell inoculation, and the surviving mice were sacrificed at this time. Bone marrow cells were analyzed by flow cytometry using anti-human CD44-FITC and Y1-Biotin/SAV-PE. Blood samples from several animals were monitored for platelet and white blood cell count. Livers were weighted and examined for tumor appearance. Other organs were also examined for tumor appearance.

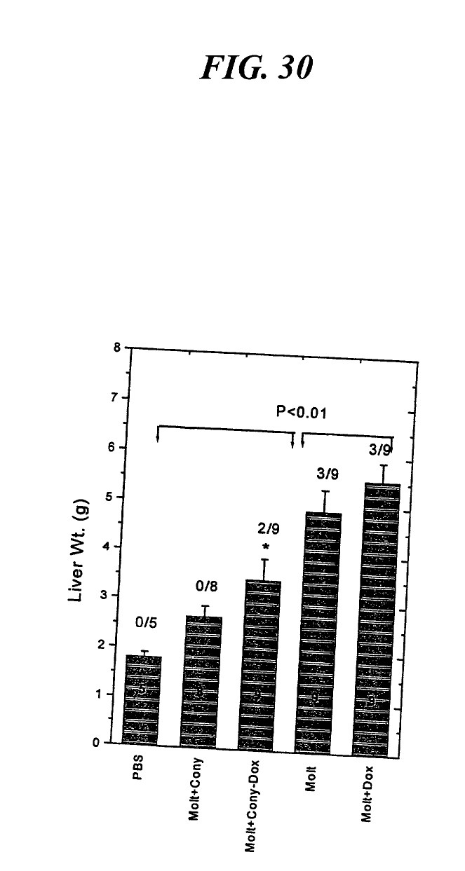

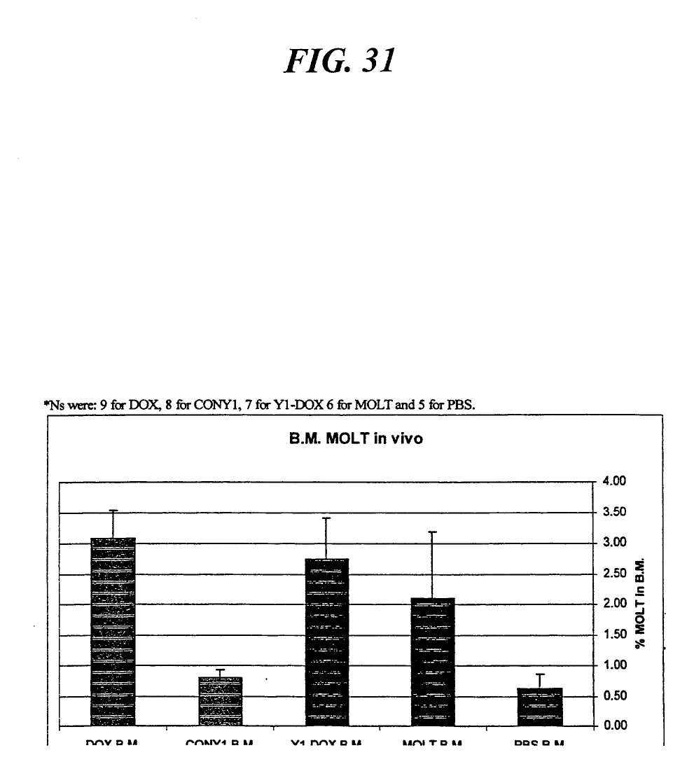

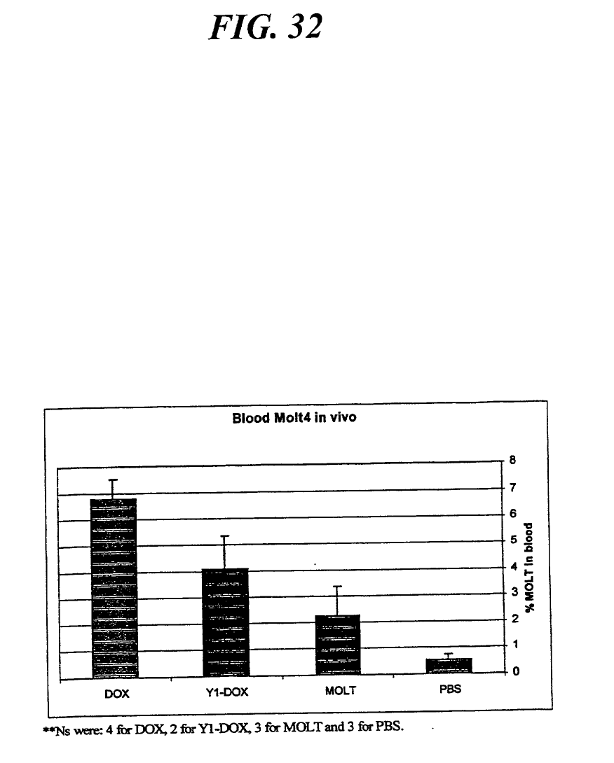

The results are depicted in (FIGS. 30, 31 and 32). Massive tumor growths (white nodules) were seen in the livers of all mice injected with MOLT-4 cells. However, livers of mice injected with MOLT-4 and treated with CONY1 or CONY1-Doxorubicin conjugate weighted significantly less than those of mice injected with MOLT-4 and treated with free Doxorubicin or left untreated. (FIG. 30).

The percentage of MOLT-4 cells found in the bone marrow was very low. (FIG. 31).

Overall, these results demonstrate that the MOLT-4 model can be used as a useful model for liver metastases of leukemia cells.

In a second experiment, SCID mice were i.v. injected with 2×10⁷ MOLT-4 cells/mouse, 5 days post treatment with cyclophosphamide. Anti-cancer agents or PBS (negative control animals) were injected i.v. three times/week from day 5 post MOLT-4 cells injection and onward. On day 35, blood was drawn from the animals, the animals were sacrificed, and their livers were excised and weighed. In the untreated, PBS-treated MOLT-4 cell-bearing animals, the liver presented with a very massive tumor growth, and its size was increased 2-3-fold relative to PBS control uninfected animals. In this experiment, there were five treatment groups:

1. PBS control, uninfected with MOLT-4 cells

2. PBS-treated MOLT-4 control

3. MOLT-4 group, treated with Y 1 scFv (CONY 1), 75 μg/mouse

4. MOLT-4 group, treated with CONY1 scFv-Doxorubicin, 75 μg/mouse

MOLT-4 group, treated with Doxorubicin, 0.1 mg/kg.

In parallel, portions of liver tissue were taken for histology and cell harvesting for FACS analysis. The survival rate of another group of treated animals was recorded relative to that of control untreated mice.

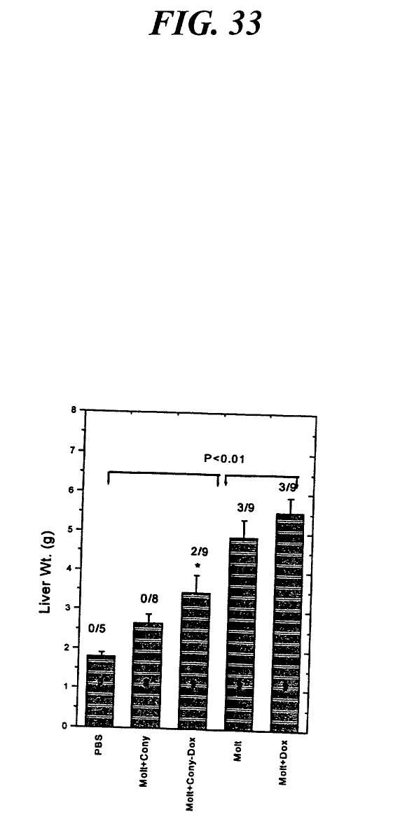

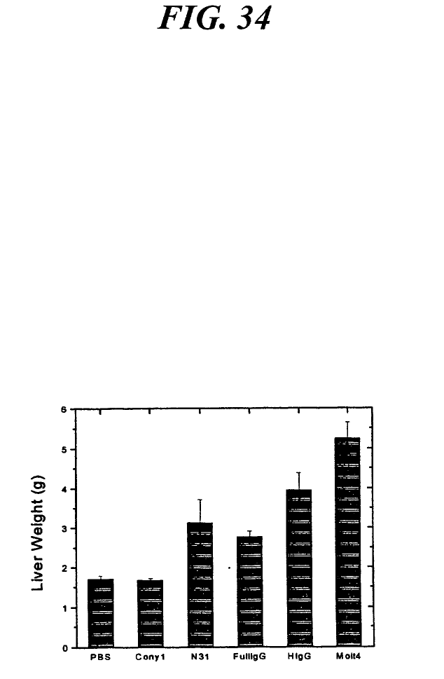

The liver weights, on day 35, are presented in (FIG. 33). As shown, liver size almost tripled in the tumor-infected mice, negative control PBS treated relative to PBS control, and non-MOLT-4-injected mice. The liver weights of mice treated with a low dose of Doxorubicin were similar to that of PBS treated tumor-infected mice. On the other hand, CONY1 scFv and CONY1 scFv-Doxorubicin conjugate treatments markedly inhibited tumor growth in the liver (much lower liver weights).

In a third experiment, using the identical SCID/MOLT-4 protocol, there were 6 groups:

1. PBS control, uninfected MOLT-4 cells

2. PBS-treated Molt control

3. Molt group, treated with CONY1 scFv, 75 μg/mouse

4. Molt group, treated with a non-specific scFv antibody derived from the Nissim I library, 75 μg/mouse (control)

5. Molt group, treated with Y1-IgG, 5 μg/mouse 6. MOLT-4 group, treated with a non-specific human-IgG, 5 μg/mouse (control)

The results shown in (FIG. 34) indicate that treatment with either CONY1 scFv or Y1 IgG inhibited tumor growth (based on liver weights), while little or no effect was seen in the animals treated with the non-specific antibody molecules.

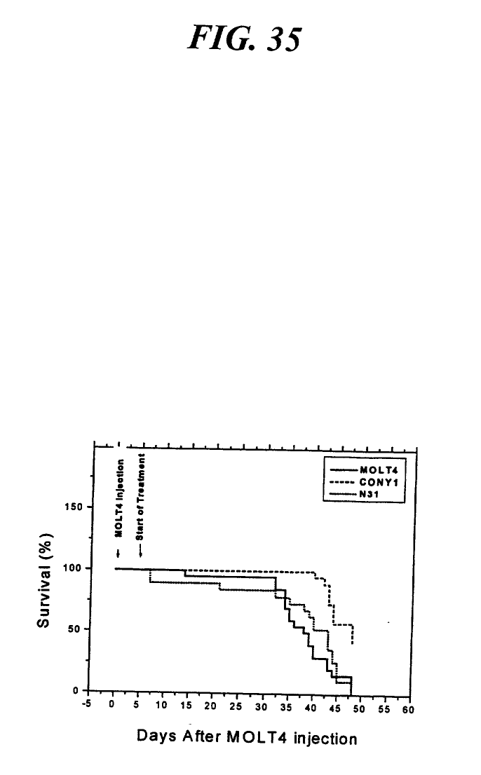

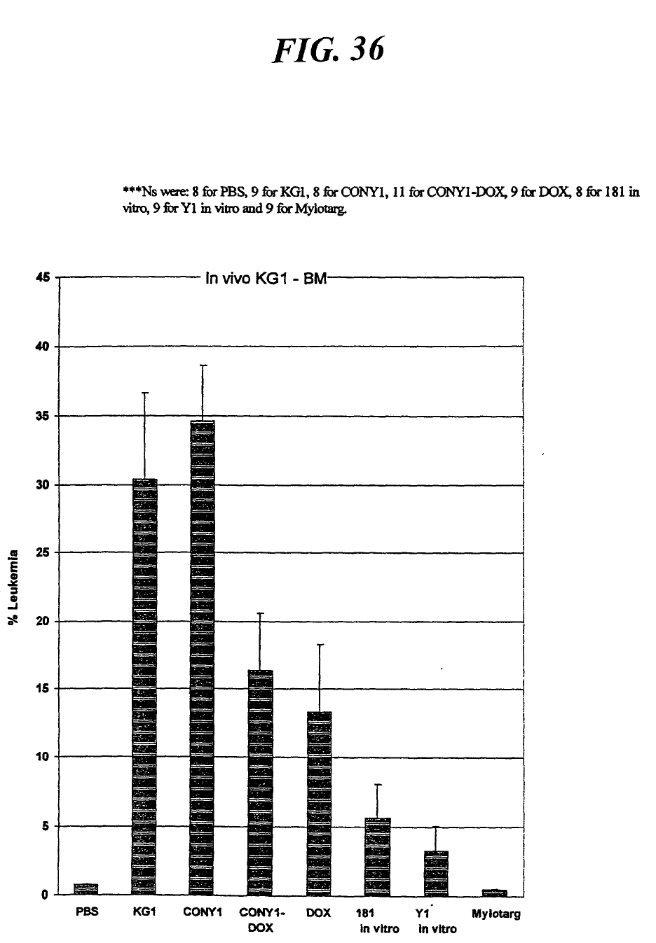

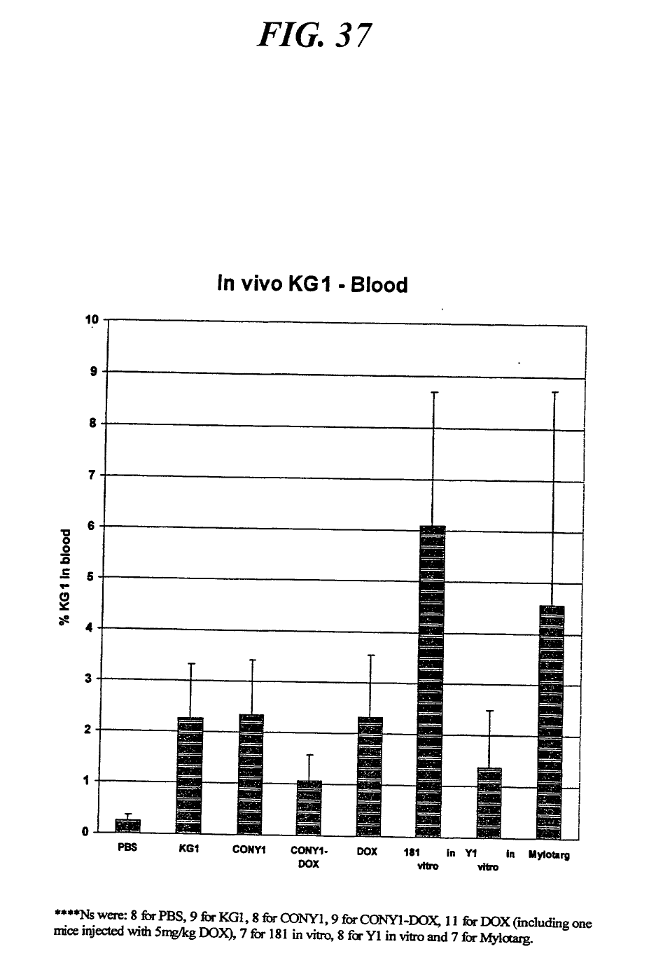

Survival was assessed in mice from three groups which received continued treatment, and the results are presented in (FIG. 35). As shown, only survival of CONY1 scFv-treated mice was prolonged.

AML KG-1 Model

The in vivo mouse model for human AML uses SCID/NOD mice (Jackson Laboratories) inoculated with KG-1 cells derived from a human AML cell line.

In a first experiment, NOD/SCID mice were pretreated with 100 mg/kg CYTOXAN®. Four days post CYTOXAN® injection, KG-1 cells were injected intravenously into the tail vein of six groups of mice. (Table N, Groups 2 and 5-9). One group of mice (Table N, Group 1) was injected with PBS alone (control). [273.][273.]

Beginning 19 days post KG-1 injection mice were treated with: CONY1, Doxorubicin, CONY1-Doxorubicin conjugate, or Mylotarg®. (Mylotarg® is a monoclonal antibody (anti CD33) conjugated chemically to calcheamicin recently approved by the FDA for treatment of AML patients age 60 and over in a first relapse.) Mice were treated once or three times per week for three weeks. One group (group 2) of KG-1 inoculated mice were left untreated. (Table N). Two other groups of mice (groups 3 and 4) were injected with KG-1 cells that were previously incubated with CONY1 or 181-scFv (a negative, non-specific control antibody) in serum free RPMI containing 1% BSA at 4° C. for 1 h. The antibodies were used at a concentration of 0.25 mg scFv/10⁸ cells (75 μg/mouse). Before injection into the mice the preincubated KG-1 cells were washed and resuspended in RPMI. The KG-1 cells in RPMI were inoculated into mice at a concentration of 75 μg scFv/0.2 ml RPMI per mouse. Group 3 mice were inoculated with KG-1+CONY1, and group 4 mice were inoculated with KG-1+181-scFv. (Table N). This treatment (group 3 and 4) was carried out one day after the inoculation of groups 1-2 and 5-9, i.e., at five days after CYTOXAN injection.

TABLE 14

| # of Mice | Group # | Inoculation | Treatment |
|---|---|---|---|
| 9 | 1 | PBS | — |
| 11 | 2 | KG-1 | — |
| 9 | 3 | KG-1 + Y1 | — |
| 9 | 4 | KG-1 + 181 | — |
| 8 | 5 | KG-1 | 75 μg/mouse (2.5 mg/kg) CONY1, 3 |
| | | | times per week |
| 9 | 6 | KG-1 | 0.1 mg/kg Doxorubicin, |
| | | | 3 times per week |
| 10 | 7 | KG-1 | 5 mg/kg Doxorubicin, |
| | | | 1 time per week |
| 11 | 8 | KG-1 | 75 μg/mouse (2.5 mg/kg) |
| | | | CONY1-Doxorubicin, 3 times |
| | | | per week |
| 9 | 9 | KG-1 | 0.2 mg/kg Mylotarg ®, |
| | | | 1 time per week |

Mice were sacrificed from 60 to 65 days post cell injection. Bone marrow and blood samples were analyzed by flow cytometry using mouse anti human CD34-FITC (IQP 144F) (or anti CD44-FITC (MCA89F, Serotec)) and Y1-Biotin/SAV-PE. Mouse IgG1-FITC (IQP 191-F) was used as an isotype control, and mouse IgG2a-FITC (MCA929F, Serotec) was used as a negative control. Flow cytometry was performed using FACSCalibur system and CellQwest software, Becton Dickinson.

The results are depicted in (FIGS. Tab 6, pages 5 and 6). Nine out of 10 KG-1 cells-injected mice that were treated with 5 mg/kg free Doxorubicin (group 7) died within three weeks after treatment initiation.

The bone marrow of mice injected with KG-1 cells that were not treated (group 2) contained about 30% KG-1 cells on average of bone marrow cell population. All mice in this group developed leukemia.

Overall, nearly all mice developed leukemia, with average of 30% KG-1 cells in the bone marrow (as determined by FACS analysis). In general, KG-1 engraftment was confined to the bone marrow. Less than 10% KG-1 cells were found in the blood In one case a solid tumor was observed on peritoneal wall.

Mice injected with KG-1 cells and treated with 0.1 mg/kg free Doxorubicin (group 6) had a statistically significant (p<0.05) lower percentage of KG-1 cells in their bone marrow, as compared to group 2.

Mice injected with KG-1 cells and treated with CONY1-Doxorubicin conjugate (group 5) had a lower percentage of KG-1 cells in their bone marrow as compared to group 2 (16.3% versus 30.4%, respectively). However, this difference was not found to be statistically significant. It was found during the experiment that the CONY1-Doxorubicin was contaminated with lipopolysaccharides (LPS). Therefore, the optimal concentrations of CONY1-Doxorubicin could not be used, and treatment was stopped before the end of the experiment.

Mice injected with KG-1 cells incubated in vitro with CONY1 or 181-scFv (groups 3 and 4, respectively) had a significantly lower percentage of KG-1 cells in their bone marrow.

The bone marrow of both mice injected with PBS only (negative control) and mice injected with KG-1 cells and treated with Mylotarg™ (group 9) was free of KG 1 cells. These results demonstrate that this in vivo model is a useful model for AML.

The overall percentage of KG-1 cells found in the blood stream of the various groups was very low overall, with high variation within the groups. It should be noted that one mouse treated with Mylotarg™ demonstrated relatively high percentage of KG-1 cells in the blood, but not in bone marrow.

Identification of human leukemia cells (KG-1 origin) in the bone marrow and in the blood stream of the mice, was performed by FACS analysis, using commercially available anti-human CD34 or CD44 antibodies in parallel with the Y1 scFv antibodies.

On the first day of analysis, there was a significant difference between mice injected with KG-1 alone (group 2), which had higher percentage of KG-1 cells in their bone marrow, as compared to mice treated with CONY1-Doxorubicin (group 8). On the third day of analysis this situation was reversed: mice from group 8 had a higher percentage of KG-1 cells in their bone marrow when compared to mice from group 2. This situation may have resulted from the following: A) choosing mice in worse physical condition in the first day, B) proliferation of KG-1 cells in mice from group 8 during the days after treatment termination, and C) the number of mice in each group was too small to generate statistically significant results.

An additional experiment was performed in which SCID-NOD mice were i.v. injected with 3×10⁴ MOLT-4 cells/mouse 5 days post treatment with cyclophosphamide. Anti-cancer agents or PBS were injected IV three times/week, from day 14 onward. On day 60, blood was drawn, then the animals were sacrificed. Bone marrow was extracted and analyzed by FACS analysis using a commercially available antiCD44 antibody for the detection of MOLT-4 cells in the mice bone marrow cell population.

This study consisted of 7 groups:

1. PBS control, uninfected with MOLT-4 cells

2. PBS-treated KG 1 control

3. KG 1 group, treated with CONY1 scFv, 75 μg/mouse

4. KG 1 group, treated with CONY1 scFv-Doxorubicin, 75 μg/mouse

5. KG 1 group, treated with Doxorubicin, 0.1 mg/kg

6. KG 1 group, treated with Doxorubicin, 3 mg/kg, once a week

7. KG 1 group, treated with Mylotarg™, 7 μg/mouse, once a week (Mylotarg™ is antibody linked to a chemotherapeutic agent, and is FDA-approved for use in leukemia patients).

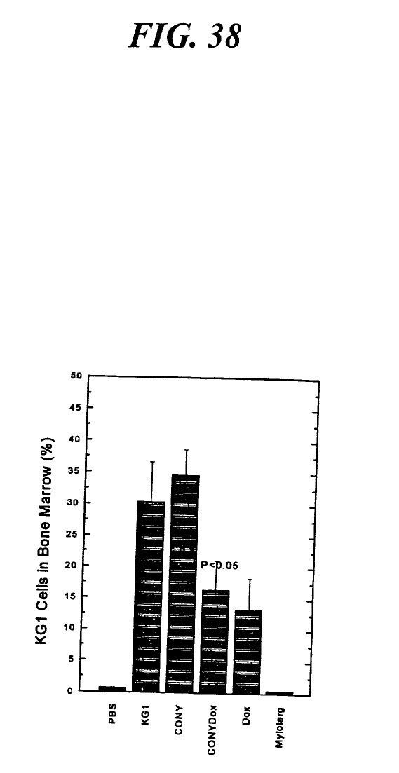

The results of the study are presented in (FIG. 38). As shown, the KG-1 cell-bearing mice had a high prevalence of cancer cells in the bone marrow. CONY1 scFv, alone, had no effect on the development of the malignancy. Mylotarg completely inhibited the prevalence of bone marrow cancer. Doxorubicin, either alone, or in the CONY1 scFv-Doxorubicin conjugate, caused a 50% reduction in the number of tumor cells in the bone marrow.

Pharmacokinetics of CONY1 in Immunosuppressed Mice

CONY 1 scFv was labeled with ¹²⁵I-Bolton Hunter reagent (to lysine). The labeling reaction was carried out at 4° C. in a borate buffer (pH 9.2) with ¹²⁵I-Bolton Hunter reagent, then ¹²⁵I-CONY1 was purified on a PD-10 chromatography column. The radioactive protein was then admixed with unlabeled CONY-1 to yield a solution of 75 μg/ml CONY-1 containing 2.5×10⁶ CPM/ml in saline.

Male Balb-C mice were pretreated by intraperitoneal injection of 0.5 ml/mouse of 0.9% NaI. After 2 hours, the mice were injected intravenously with 0.2 ml of the labeled CONY-1 solution, resulting in a ¹²⁵I-CONY-1 dose of 15 μg (5×10⁵ CPM) per mouse.

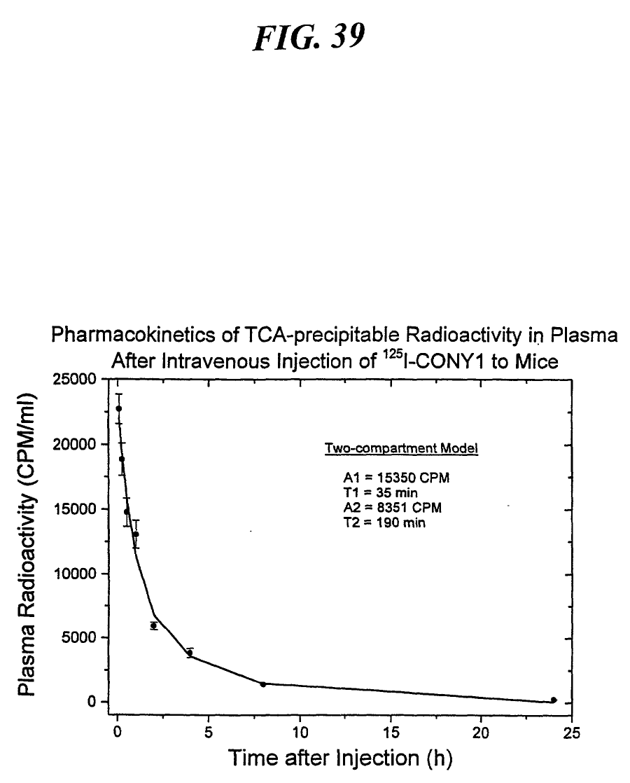

At various times after injection, blood was collected over EDTA, mice were sacrificed, and tissues were excised. Samples and organs were taken at 5, 15, and 30 minutes and at 1, 2, 4, 8, and 24 hours after injection. Two to four mice were used per time point. Plasma was separated and either counted for gamma radioactivity or subjected to precipitation with trichloroacetic acid (TCA). After centrifugation, TCA precipitates were subjected to gamma radioactivity counting. Liver, lung, kidney, spleen, and bone marrow samples were weighed and counted for gamma radioactivity. Plasma TCA precipitated radioactivity was plotted against time, and a two-compartment kinetics model was fitted. Organ/tissue total and specific radioactivity values were calculated. The results are shown in (FIGS. 39, 40 and 41).

Comparison of the blood and plasma radioactivity values indicated that practically all of the CONY-1 resided in the plasma and did not adhere to erythrocytes. The plasma radioactivity values were similar to those of the TCA precipitates, indicating that they were associated with undegraded protein. FIG. 39 shows CONY-1 levels in the plasma at various time points after administration. The values were fitted statistically to a two-compartment model, and the half-life values of blood clearance obtained were 35 and 190 minutes, respectively.

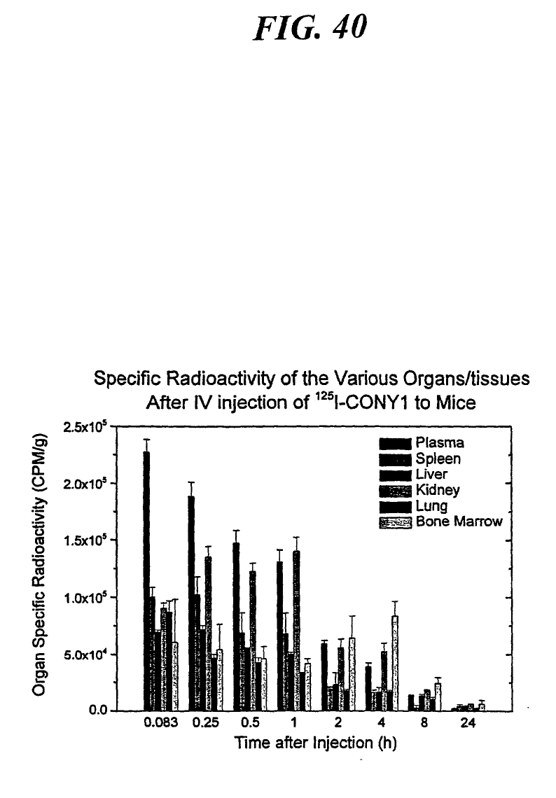

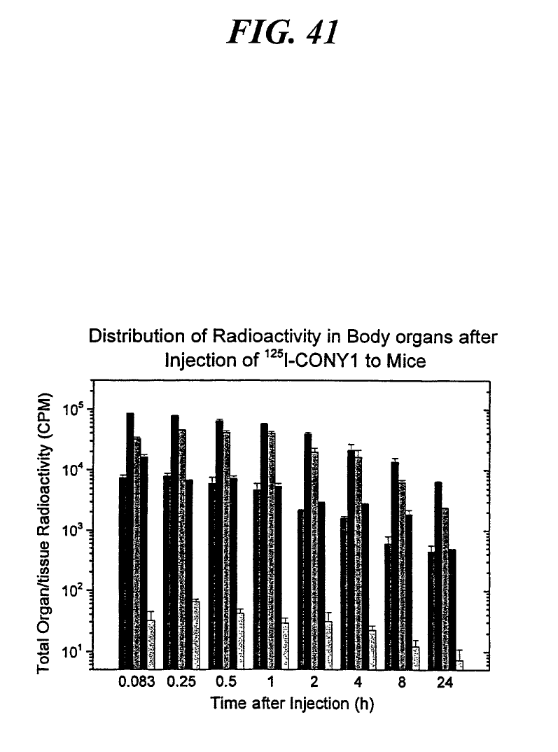

The distribution of radioactivity in various tissues at the specified time points after administration are shown as specific and total radioactivity in (FIGS. 40 and 41), respectively. In most tissues, there was no specific accumulation of radioactivity, as is evident from the comparison of the specific activity to that of the blood. Slightly higher values were seen in the kidney at 4 hours and in the bone marrow at 4 and 8 hours; this is most probably related to the excretion of degradation products.

The results indicate that CONY-1 is excreted in mice at a half-life of 35 minutes. The second compartment excretion rate is of minor importance and may be the result of the presence of some polymeric forms of the injected material. There is no major specific uptake of CONY-1 in body tissues, with the exception of a slight increase in the bone marrow.

Production of Antibodies and Fragments

Antibodies, fragments thereof, constructs thereof, peptides, polypeptides, proteins, and fragments and constructs thereof can be produced in either prokaryotic or eukaryotic expression systems. Methods for producing antibodies and fragments in prokaryotic and eukaryotic systems are well-known in the art.

A eukaryotic cell system, as defined in the present invention and as discussed, refers to an expression system for producing peptides or polypeptides by genetic engineering methods, wherein the host cell is a eukaryote. A eukaryotic expression system may be a mammalian system, and the peptide or polypeptide produced in the mammalian expression system, after purification, is preferably substantially free of mammalian contaminants. Other examples of a useful eukaryotic expression system include yeast expression systems.

A preferred prokaryotic system for production of the peptide or polypeptide of the invention uses E. coli as the host for the expression vector. The peptide or polypeptide produced in the E. coli system, after purification, is substantially free of E. coli contaminating proteins. Use of a prokaryotic expression system may result in the addition of a methionine residue to the N-terminus of some or all of the sequences provided for in the present invention. Removal of the N-terminal methionine residue after peptide or polypeptide production to allow for fall expression of the peptide or polypeptide can be performed as is known in the art, one example being with the use of Aeromonas aminopeptidase under suitable conditions (U.S. Pat. No. 5,763,215).

Types of Antibody Fragments and Constructs

The present invention provides for a peptide or polypeptide comprising an antibody or antigen binding fragment thereof, a construct thereof, or a construct of a fragment. Antibodies according to the present invention include IgG, IgA, IgD, IgE, or IgM antibodies. The IgG class encompasses several sub-classes including IgG₁, IgG₂, IgG₃, and IgG₄.

Antibody fragments according to the present invention include Fv, scFv, dsFv, Fab, Fab₂, and Fd molecules. Smaller antibody fragments, such as fragments of Fv's, are also included in the term “fragments”, as long as they retain the binding characteristics of the original antibody or larger fragment. Examples of such fragments would be (1) a minibody, which comprises a fragment of the heavy chain only of the Fv, (2) a microbody, which comprises a small fractional unit of antibody heavy chain variable region (PCT Application No. PCT/IL99/00581), (3) similar bodies comprising a fragment of the light chain, and (4) similar bodies comprising a functional unit of a light chain variable region. Constructs include, for example, multimers such as diabodies, triabodies, and tetrabodies. The phrases “antibody, binding fragment thereof, or complex comprising an antibody or binding fragment thereof” and “antibody or fragment” are intended to encompass all of these molecules, as well as derivatives and homologs, mimetics, and variants thereof, unless it is specified otherwise or indicated otherwise based on context and/or knowledge in the art.

Once an antibody, fragment, or construct having desired binding capabilities has been selected and/or developed, it is well within the ability of one skilled in the art using the guidance provided herein to produce constructs and fragments which retain the characteristics of the original antibody. For example, entire antibody molecules, Fv fragments, Fab fragments, Fab₂ fragments, dimers, trimers, and other constructs can be made which retain the desired characteristics of the originally selected or developed antibody, fragment, or construct.

If it is desired to substitute amino acids but still retain the characteristics of an antibody or fragment, it is well within the skill in the art to make conservative amino acid substitutions. Modifications such as conjugating to pharmaceutical or diagnostic agents, may also be made to antibodies or fragments without altering their binding characteristics. Other modifications, such as those made to produce more stable antibodies or fragments may also be made to antibodies or fragments without altering their specificity. For example, peptoid modification, semipeptoid modification, cyclic peptide modification, N terminus modification, C terminus modification, peptide bond modification, backbone modification, and residue modification may be performed. It is also within the ability of the skilled worker following the guidance of the present specification to test the modified antibodies or fragments to assess whether their binding characteristics have been changed.

Likewise, it is within the ability of the skilled worker using the guidance provided herein to alter the binding characteristics of an antibody, fragment, or construct to obtain a molecule with more desirable characteristics. For example, once an antibody having a desirable properties is identified, random or directed mutagenesis may be used to generate variants of the antibody, and those variants may be screened for desirable characteristics.

Antibodies and fragments according to the present invention may also have a tag may be inserted or attached thereto to aid in the preparation and identification thereof, and in diagnostics. The tag can later be removed from the molecule. Examples of useful tags include: AU1, AU5, BTag, c-myc, FLAG, Glu-Glu, HA, His6, HSV, HTTPHH, IRS, KT3, Protein C, S-Tag®, T7, V5, and VSV-G (Jarvik and Telmer, Ann. Rev. Gen., 32, 601-618 (1998)). The tag is preferably c-myc.

Multimeric Antibodies

The present invention provides for a Y1 or Y17 peptide or polypeptide comprising an scFv molecule. As used herein a scFv is defined as a molecule which is made up of a variable region of a heavy chain of a human antibody and a variable region of a light chain of a human antibody, which may be the same or different, and in which the variable region of the heavy chain is connected, linked, fused or covalently attached to, or associated with, the variable region of the light chain.

A Y1 and Y17 scFv construct may be a multimer (e.g., dimer, trimer, tetramer, and the like) of scFv molecules that incorporate one or more of the hypervariable domains of the Y1 or Y17 antibody. All scFv derived constructs and fragments retain enhanced binding characteristics so as to bind selectively and/or specifically to a target cell in favor of other cells. The binding selectivity and/or specificity is primarily determined by hypervariable regions.

The hypervariable loops within the variable domains of the light and heavy chains are termed Complementary Determining Regions (CDR). There are CDR1, CDR2 and CDR3 regions in each of the heavy and light chains. The most variable of these regions is the CDR3 region of the heavy chain. The CDR3 region is understood to be the most exposed region of the Ig molecule, and as provided herein, is the site primarily responsible for the selective and/or specific binding characteristics observed.

The Y1 and Y17 peptide of the subject invention can be constructed to fold into multivalent Fv forms. Y1 and Y17 multimeric forms were constructed to improve binding affinity and specificity and increased half-life in blood.

Mulitvalent forms of scfv have been produced by others. One approach has been to link two scFvs with linkers. Another approach involves using disulfide bonds between two scFvs for the linkage. The simplest approach to production of dimeric or trimeric Fv was reported by Holliger et al., PNAS, 90, 6444-6448 (1993) and A. Kortt, et al., Protein Eng., 10, 423-433 (1997). One such method was designed to make dimers of scFvs by adding a sequence of the FOS and JUN protein region to form a leucine zipper between them at the c-terminus of the scFv. Kostelny S A et al., J Immunol. Mar. 1, 1992; 148(5):1547-53; De Kruif J et al., J Biol Chem. Mar. 29, 1996; 271(13):7630-4. Another method was designed to make tetramers by adding a streptavidin coding sequence at the c-terminus of the scFv. Streptavidin is composed of 4 subunits so when the scFv-streptavidin is folded, 4 subunits accommodate themselves to form a tetramer. Kipriyanov S M et al., Hum Antibodies Hybridomas, 1995;6(3):93-101. In yet another method, to make dimers, trimers and tetramers, a free cysteine is introduced in the protein of interest. A peptide-based cross linker with variable numbers (2 to 4) of maleimide groups was used to cross link the protein of interest to the free cysteines. Cochran J R et al., Immunity, March 2000; 12(3):241-50.

In this system, the phage library (as described herein above) was designed to display scFvs, which can fold into the monovalent form of the Fv region of an antibody. Further, and also discussed herein above, the construct is suitable for bacterial expression. The genetically engineered scFvs comprise heavy chain and light chain variable regions joined by a contiguously encoded 15 amino acid flexible peptide spacer. The preferred spacer is (Gly₄Ser)₃. The length of this spacer, along with its amino acid constituents provides for a nonbulky spacer, which allows the V_{H} and the V_{L} regions to fold into a functional Fv domain that provides effective binding to its target.

The present invention is directed to Y1 and Y17 multimers prepared by any known method in the art. A preferred method of forming multimers, and especially dimers, employs the use of cysteine residues to form disulfide bonds between two monomers. In this embodiment, dimers are formed by adding a cysteine on the carboxyl terminus of the scFvs (referred to as Y1-cys scFv or Y1 dimer) in order to facilitate dimer formation. After the DNA construct was made (See Example 2D and 6D) and used for transfection, Y1 dimers were expressed in a production vector and refolded in vitro. The protein was analyzed by SDS-PAGE, HPLC, and FACS. However, none of these first attempts indicated that a dimer formed. Thus, the process was repeated and this time, two-liter fermentation batches of the antibodies were run. After expressing Y1-cys in E. coli strain BL21, refolding was done in arginine. Following refolding, the protein was dialyzed and purified by Q-sepharose and gel filtration (sephadex 75). Two peaks were detected by SDS-PAGE (non-reduced) and by gel filtration. The peaks were collected separately and analyzed by FACS. Monomer and dimer binding to Jurkat cells was checked by FACS. The binding by dimers required only 1/100 the amount of the monomeric antibody for the same level of staining, indicating that the dimer has greater avidity. Conditions for dimer refolding were determined, and material comprising >90% dimers (mg quantities) was produced after subsequent dialysis, chromatographic, and gel filtration steps. The purified dimer was characterized by gel filtration and by SDS-PAGE analysis under oxidizing conditions. The dimer's binding capacity was confirmed by radioreceptor assay, ELISA, and FACS analyses.

To compare the binding of the scFv monomer (also referred to as CONY1) with the Y1 dimer, binding competition experiments were done in vitro on KG-1 cells. In addition, these experiments also compared the binding of the full Y1 IgG to the scFv Y1 monomers. To perform this study, a Y1 IgG was labeled with biotin. This study revealed that Y1 IgG competed with IgG Y1-Biotin. Non-relevant human IgG did not compete with the labeled Y1 IgG. Y1 scFvs (5 μg and 10 μg) partially competed with Y1 IgG-Biotin (50 ng). The studies also showed that 1 ng of IgGY1-FITC bound to KG-1 cells (without serum) to the same extent as 1 μg of Y1 scFv-FITC, but in the presence of serum, most of this binding was blocked. These studies also showed that the binding of the Y1 dimer is at least 20-fold higher than that of the Y1 scFv monomer as analyzed by radioreceptor assay, ELISA or FACS.

In yet another embodiment, a lysine-alanine-lysine was added in addition to the cysteine at the carboxyl end (referred to as Y1-cys-kak scFv). The amino acid sequence of this scFv construct is reproduced below and is also SEQ ID NO: 212.

| | | |
|---|---|---|
| 1 | MEVQLVESGG GVVRPGGSLR LSCAASGFTF DDYGMSWVRQ APGKGLEWVS GINWNGGSTG | 60 |
| 61 | YADSVKGRFT ISRDNAKNSL YLQMNSLRAE DTAVYYCARM RAPVIWGQGT LVTVSRGGGG | 120 |
| 121 | SGGGGSGGGG SSELTQDPAV SVALGQTVRI TCQGDSLRSY YASWYQQKPG QAPVLVIYGK | 180 |
| 181 | NNRPSGIPDR FSGSSSGNTA SLTITGAQAE DEADYYCNSR DSSGNHVVFG GGTKLTVLGG | 240 |
| 241 | GGCKAK | |

The Y1-cys-kak was produced in a λ-pL vector in bacteria. Expression in the λ-pL vector was induced by increasing the temperature to 42° C. Inclusion bodies were obtained from induced cultures and semi-purified by aqueous solutions, to remove unwanted soluble proteins. The inclusion bodies were solubilized in guanidine, reduced by DTE, and refolded in vitro in a solution based on arginine/ox-glutathione. After refolding, the protein was dialyzed and concentrated by tangential flow filtration to a buffer containing Urea/phosphate buffer. The protein was repurified and concentrated by ionic-chromatography in an SP-column.

The dimer was characterized by SDS-page electrophoresis, gel filtration chromatography, ELISA, radioreceptor binding, and FACS. The apparent affinity of the dimer was higher than the monomer due to the avidity effect. This effect was confirmed by ELISA to glycocalicin, FACS to KG-1 cells, and competition in a radioreceptor assay.

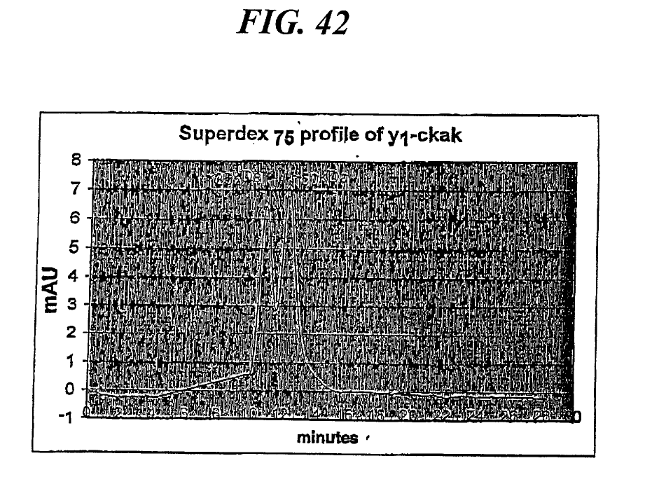

HPLC was performed to profile the dimer after refolding and purification from a Superdex 75 gel filtration column. In FIG. 42, the Y1-cys-kak (dimer) is the first peak on the left (˜10.8 minutes) and the subsequent peak is the monomer (˜12 minutes). The dimer is approximately 52 kDa and the monomer 26 kDa, according to protein size markers run on the same column. The balance between the dimer and monomer can be changed by varying the conditions of the refolding (concentration of the oxidized agent and the concentration of the protein in the refolding buffer). The dimer and monomer were separated by chromatography in a superdex 75 column.

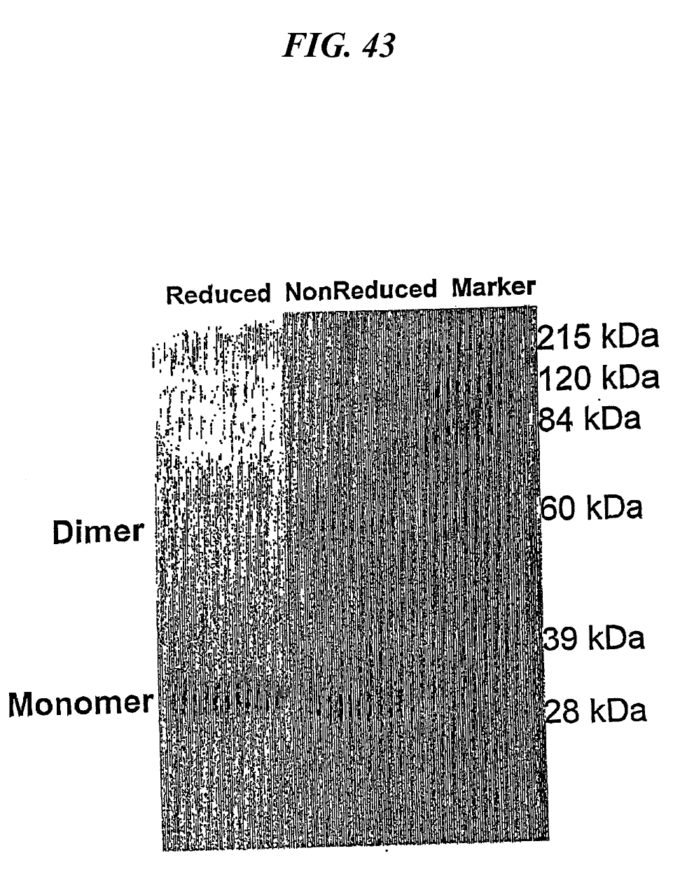

In FIG. 43, a gel is shown with a mixed population of dimers and monomers. In the reduced form, the monomers are seen due to the reduction between the two monomers and in the non reduced form, two population are seen (as in the gel filtration experiment) a monomer fraction of about 30 kDa and a dimer of about 60 kDa.

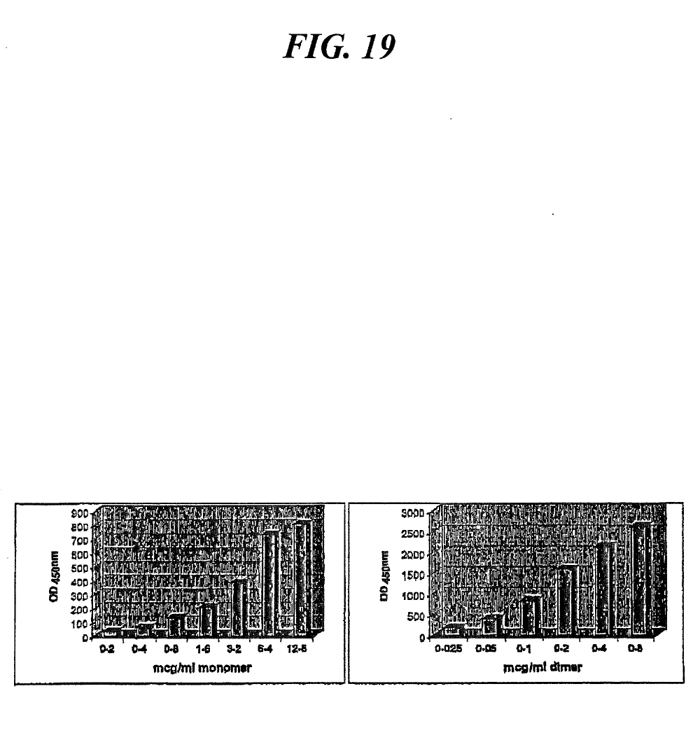

An ELISA assay was performed to ascertain the differences in binding between the monomer (the Y1 scFv-also known as Y1-kak) and the dimer Y1-cys-kak (the cysteine dimer) for antigen GPIb (glycocalicin) derived from platelets. A polyclonal anti single chain antibody and/or a novel polyclonal anti-V_{L} (derived from rabbits) and anti-rabbit HRP, were used to detect the binding to GPIb. The dimer was approximately 100 fold more active than the monomer. For instance, to reach 0.8 OD units 12.8 μg/ml of monomer was used compared to only 0.1 μg/ml of dimer. See FIG. 19.

In addition, FACS binding analysis to KG-1 cells showed that the dimer is more sensitive than the monomer when a two or three step binding assay was performed. Dimers directly labeled by FITC showed a slight advantage (use of 10× fold less material) than the monomer. The radio receptor assay on KG-1 cells, where the dimer was used as competitor, showed that the dimer is 30× fold more efficient than the monomer.

Varying the length of the spacers is yet another preferred method of forming dimers, trimers, and triamers (often referred to in the art as diabodies, triabodies and tetrabodies, respectively). Dimers are formed under conditions where the spacer joining the two variable chains of a scFv is shortened to generally 5-12 amino acid residues. This shortened spacer prevents the two variable chains from the same molecule from folding into a functional Fv domain. Instead, the domains are forced to pair with complimentary domains of another molecule to create two binding domains. In a preferred method, a spacer of only 5 amino acids (Gly₄Ser) was used for diabody construction. This dimer can be formed from two identical scFvs, or from two different populations of scFvs and retain the selective and/or specific enhanced binding activity of the parent scFv(s), and/or show increased binding strength or affinity.

In a similar fashion, triabodies are formed under conditions where the spacer joining the two variable chains of a scFv is shortened to generally less than 5 amino acid residues, preventing the two variable chains from the same molecule from folding into a functional Fv domain. Instead, three separate scFv molecules associate to form a trimer. In a preferred method, triabodies were obtained by removing this flexible spacer completely. The triabody can be formed from three identical scFvs, or from two or three different populations of scFvs and retain the selective and/or specific enhanced binding activity of the parent scFv(s), and/or show increased binding strength or affinity.

Tetrabodies are similarly formed under conditions where the spacer joining the two variable chains of a scFv is shortened to generally less than 5 amino acid residues, preventing the two variable chains from the same molecule from folding into a functional Fv domain. Instead, four separate scFv molecules associate to form a tetramer. The tetrabody can be formed from four identical scFvs, or from 1-4 individual units from different populations of scFvs and should retain the selective and/or specific enhanced binding activity of the parent scFv(s), and/or show increased binding strength or affinity.

Whether triabodies or tetrabodies form under conditions where the spacer is generally less than 5 amino acid residues long depends on the amino acid sequence of the particular scFv(s) in the mixture and the reaction conditions.

In a preferred method, tetramers are formed via a biotin/streptavidin association. A novel fermentation construct that is capable of being enzymatically labeled with biotin (referred to herein as Y1-biotag or Y1-B) was created. A sequence that is a substrate for the BirA enzyme was added at the Y1 C-terminus. The BirA enzyme adds a biotin to the lysine residue within the sequence. Y1-biotag was expressed in E. coli. The inclusion body material was isolated and refolded. The purity of the folded protein was >95%, and >100 mg were obtained from a 1-L culture (small-scale, non-optimized conditions). The molecular weight of this form was found to be similar to that of the scFv according to HPLC, SDS-PAGE, and mass spectroscopy. Y1-biotag was found to be the most consistent reagent for FACS analysis. However, when Y1-biotag binding to KG-1 cells was examined in the presence of serum, high concentrations (10-fold more) are required for comparable binding in the absence of serum. Nevertheless, this construct offered the advantage of specific biotinylation in which the binding site of the molecule remains intact. Further, each molecule is labeled by only one biotin—each molecule receives one biotin on the carboxyl end.

Limiting labeling to one biotin/molecule in a desired location enabled production of tetramers with streptavidin. The tetramers were formed by incubating Y1-B with steptavidin-PE.

FACS analysis indicated that the tetramers made by Y1-biotag and streptavidin-PE were 100 to 1000 fold more sensitive that Y1 scFv monomers. Y1-biotag tetramers with streptavidin-PE appear to specifically bind to one of the Y1-reactive cell lines (KG-1). The differential of this reaction, from background binding, was very high, and offered high sensitivity to detect low amounts of receptor. FACS evaluation of normal whole blood with Y1BSAV tetramers indicated that no highly reactive population is present. Monocytes and granulocytes were positive to a small extent. In cell lines where a positive result was present, such as with KG-1 cells, the tetramers were at least 100-fold more reactive.

Then, the tetramers were incubated with the cell samples. A low dose of the Y1 tetramers (5 ng) binds well to the cell line (KG-1) providing a 10 to 20-fold higher response than previously observed with other Y1 antibody forms. A minor reaction was observed when a negative cell line was examined with varying doses of the tetramers.

Conjugates for Diagnostic and Pharmaceutical Use

The antibodies and binding fragments thereof of the subject invention can be associated with, combined, fused or linked to various pharmaceutical agents, such as drugs, toxins, and radioactive isotopes with, optionally, a pharmaceutically effective carrier, to form drug-peptide compositions, fusions or conjugates having anti-disease and/or anti-cancer activity. Such conjugates and fusions may also be used for diagnostic purposes.

Examples of carriers useful in the invention include dextran, HPMA (a lipophilic polymer) or any other polymer. Alternatively, decorated liposomes can be used, such as liposomes decorated with scFv Y1 molecules, such as Doxil, a commercially available liposome containing large amounts of doxorubicin. Such liposomes can be prepared to contain one or more desired pharmaceutical agents and be admixed with the antibodies of the present invention to provide a high drug to antibody ratio.

Alternatively, the link between the antibody or fragment thereof and the pharmaceutical agent may be a direct link. A direct link between two or more neighboring molecules may be produced via a chemical bond between elements or groups of elements in the molecules. The chemical bond can be for example, an ionic bond, a covalent bond, a hydrophobic bond, a hydrophilic bond, an electrostatic bond or a hydrogen bond. The bonds can be, for example, amine, carboxy, amide, hydroxyl, peptide and/or disulfide bonds. The direct link may preferably be a protease resistant bond.

The link between the peptide and the pharmaceutical agent or between the peptide and carrier, or between the carrier and pharmaceutical agent may be via a linker compound. As used herein in the specification and in the claims, a linker compound is defined as a compound that joins two or more moieties together. The linker can be straight-chained or branched. A branched linker compound may be composed of a double-branch, triple branch, or quadruple or more branched compound. Linker compounds useful in the present invention include those selected from the group comprising dicarboxylic acids, malemido hydrazides, PDPH, carboxylic acid hydrazides, and small peptides.

More specific examples of linker compounds useful according to the present invention, include:

a. Dicarboxylic acids such as succinic acid, glutaric acid, and adipic acid;

b. Maleimido hydrazides such as N-[□-maleimidocaproic acid] hydrazide, 4-[N-maleimidomethyl]cyclohexan-1-carboxylhydrazide, and N-[□-maleimidoundcanoic acid] hydrazide;

c. PDPH linkers such as (3-[2-pyridyldithio]propionyl hydrazide) conjugated to sulfurhydryl reactive protein; and

d. Carboxylic acid hydrazides selected from 2-5 carbon atoms.

Linking via direct coupling using small peptide linkers is also useful. For example, direct coupling between the free sugar of, for example, the anti-cancer drug doxorubicin and a scFv may be accomplished using small peptides. Examples of small peptides include AU1, AU5, BTag, c-myc, FLAG, Glu-Glu, HA, His6, HSV, HTTPHH, IRS, KT3, Protein C, S-Tag®, T7, V5, and VSV-G.

Antibodies, and fragments thereof, of the present invention may be bound to, conjugated to, complexed with or otherwise associated with imaging agents (also called indicative markers), such as radioisotopes, and these conjugates can be used for diagnostic and imaging purposes. Kits comprising such radioisotope-antibody (or fragment) conjugates are provided.

Examples of radioisotopes useful for diagnostics include ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}telluriunm ¹⁶⁵thulium, ¹⁶⁷thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵rutruthenium, ⁹⁷ruthenium, ¹⁰³ruthenium, ¹⁰⁵ruthenium, ¹⁰⁷mercury, ²⁰³mercury, ⁶⁷gallium and ⁶⁸gallium. Preferred radioactive isotopes, are opaque to X-rays or paramagnetic ions.

The indicative marker molecule may also be a fluorescent marker molecule. Examples of fluorescent marker molecules include fluorescein, phycoerythrin, or rhodamine, or modifications or conjugates thereof.

Antibodies or fragments conjugated to indicative markers may be used to diagnose or monitor disease states. Such monitoring may be carried out in vivo, in vitro, or ex vivo. Where the monitoring or diagnosis is carried out in vivo or ex vivo, the imaging agent is preferably physiologically acceptable in that it does not harm the patient to an unacceptable level. Acceptable levels of harm may be determined by clinicians using such criteria as the severity of the disease and the availability of other options.

The present invention provides for a diagnostic kit for in vitro analysis of treatment efficacy before, during, or after treatment, comprising an imaging agent comprising a peptide of the invention linked to an indicative marker molecule, or imaging agent. The invention further provides for a method of using the imaging agent for diagnostic localization and imaging of a cancer, more specifically a tumor, comprising the following steps:

a) contacting the cells with the composition,

b) measuring the radioactivity bound to the cells, and hence

c) visualizing the tumor.

Examples of suitable imaging agents include fluorescent dyes, such as FITC, PE, and the like, and fluorescent proteins, such as green fluorescent proteins. Other examples include radioactive molecules and enzymes that react with a substrate to produce a recognizable change, such as a color change.

In one example, the imaging agent of the kit is a fluorescent dye, such as FITC, and the kit provides for analysis of treatment efficacy of cancers, more specifically blood-related cancers, e.g., leukemia, lymphoma and myeloma. FACS analysis is used to determine the percentage of cells stained by the imaging agent and the intensity of staining at each stage of the disease, e.g., upon diagnosis, during treatment, during remission and during relapse.

Antibodies, and fragments thereof, of the present invention may be bound to, conjugated to, or otherwise associated with anti-cancer agents, anti-leukemic agents, anti-viral agents, anti-metastatic agents, anti-inflammatory agents, anti-thrombosis agents, anti-restenosis agents, anti-aggregation agents, anti-autoimmune agents, anti-adhesion agents, anti-cardiovascular disease agents, or other anti-disease agents or pharmaceutical agent. A pharmaceutical agent refers to an agent that is useful in the prophylactic treatment or diagnosis of a mammal including, but not restricted to, a human, bovine, equine, porcine, murine, canine, feline, or any other warm-blooded animal.

Examples of such pharmaceutical agents include, but are not limited to anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

Other exemplary pharmaceutical agents include doxorubicin, methoxymorpholinyldoxorubicin (morpholinodoxorubicin), adriamycin, cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide and bleomycin, and derivatives and combinations thereof

An anti-cancer agent is an agent with anti-cancer activity. For example, anti-cancer agents include agents that inhibit or halt the growth of cancerous or immature pre-cancerous cells, agents that kill cancerous or pre-cancerous, agents that increase the susceptibility of cancerous or pre-cancerous cells to other anti-cancer agents, and agents that inhibit metastasis of cancerous cells. In the present invention, an anti-cancer agent may also be agent with anti-angiogenic activity that prevents, inhibits, retards or halts vascularization of tumors.

Inhibition of growth of a cancer cell includes, for example, the (i) prevention of cancerous or metastatic growth, (ii) slowing down of the cancerous or metastatic growth, (iii) the total prevention of the growth process of the cancer cell or the metastatic process, while leaving the cell intact and alive, or (iv) killing the cancer cell.

An anti-leukemia agent is an agent with anti-leukemia activity. For example, anti-leukemia agents include agents that inhibit or halt the growth of leukemic or immature pre-leukemic cells, agents that kill leukemic or pre-leukemic, agents that increase the susceptibility of leukemic or pre-leukemic cells to other anti-leukemia agents, and agents that inhibit metastasis of leukemic cells. In the present invention, an anti-leukemia agent may also be agent with anti-angiogenic activity that prevents, inhibits, retards or halts vascularization of tumors.

Inhibition of growth of a leukemia cell includes, for example, the (i) prevention of leukemic or metastatic growth, (ii) slowing down of the leukemic or metastatic growth, (iii) the total prevention of the growth process of the leukemia cell or the metastatic process, while leaving the cell intact and alive, or (iv) killing the leukemia cell.

Examples of anti-disease, anti-cancer, and anti-leukemic agents to which antibodies and fragments of the present invention may usefully be linked include toxins, radioisotopes, and pharmaceuticals.

Examples of toxins include gelonin, Pseudomonas exotoxin (PE), PE40, PE38, diphtheria toxin, ricin, or modifications or derivatives thereof.

Examples of radioisotopes include gamma-emitters, positron-emitters, and x-ray emitters that may be used for localization and/or therapy, and beta-emitters and alpha-emitters that may be used for therapy.

More specific examples of therapeutic radioisotopes include ¹¹¹indium, ¹¹³indium, ^{99m}rhenium, ¹⁰⁵rhenium, ¹⁰¹rhenium, ^{99m}technetium, ^{121m}tellurium, ^{122m}tellurium ^{125m}telluriunm ¹⁶⁵thulium, ¹⁶⁷thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵¹⁰⁷rthenium, ¹⁰⁷mercury, ²⁰³mercury, ⁶⁷gallium and ⁶⁸gallium.

Non-limiting examples of anti-cancer or anti-leukemia pharmaceutical agents include doxorubicin, adriamycin, cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide and bleomycin, and derivatives thereof, an combinations thereof.

Pharmaceutical Compositions

Antibodies, constructs, conjugates, and fragments of the subject invention may be administered to patients in need thereof via any suitable method. Exemplary methods include intravenous, intramuscular, subcutaneous, topical, intratracheal, intrathecal, intraperitoneal, intralymphatic, nasal, sublingual, oral, rectal, vaginal, respiratory, buccal, intradermal, transdermal or intrapleural administration.

For intravenous administration, the formulation preferably will be prepared so that the amount administered to the patient will be an effective amount from about 0.1 mg to about 1000 mg of the desired composition. More preferably, the amount administered will be in the range of about 1 mg to about 500 mg of the desired composition. The compositions of the invention are effective over a wide dosage range, and depend on factors such as the particulars of the disease to be treated, the half-life of the peptide or polypeptide-based pharmaceutical composition in the body of the patient, physical and chemical characteristics of the pharmaceutical agent and of the pharmaceutical composition, mode of administration of the pharmaceutical composition, particulars of the patient to be treated or diagnosed, as well as other parameters deemed important by the treating physician.

Pharmaceutical composition for oral administration may be in any suitable form. Examples include tablets, liquids, emulsions, suspensions, syrups, pills, caplets, and capsules. Methods of making pharmaceutical compositions are well known in the art. See, e.g., Remington, The Science and Practice of Pharmacy, Alfonso R. Gennaro (Ed.) Lippincott, Williams & Wilkins (pub).

The pharmaceutical composition may also be formulated so as to facilitate timed, sustained, pulsed, or continuous release. The pharmaceutical composition may also be administered in a device, such as a timed, sustained, pulsed, or continuous release device.

The pharmaceutical composition for topical administration can be in any suitable form, such as creams, ointments, lotions, patches, solutions, suspensions, and gels.

Compositions comprising antibodies, constructs, conjugates, and fragments of the subject invention may comprise conventional pharmaceutically acceptable diluents, excipients, carriers, and the like. Tablets, pills, caplets and capsules may include conventional excipients such as lactose, starch and magnesium stearate. Suppositories may include excipients such as waxes and glycerol. Injectable solutions comprise sterile pyrogen-free media such as saline, and may include buffering agents, stabilizing agents or preservatives. Conventional enteric coatings may also be used.

### EXAMPLES

The following examples are set forth to aid in understanding the invention but are not intended and should not be construed, to limit its scope in any way. Although specific reagents and reaction conditions are described, modifications can be made that are meant to be encompassed by the scope of the invention. The following examples, therefore, are provided to further illustrate the invention.

### Example 1

### Preparation of Platelets

1.1 Preparation of Washed Platelets.

Platelet concentrate in acid-citrate-dextrose (ACD) was obtained from a blood bank, platelets were isolated, washed once in buffer containing ACD and saline in a ratio of 1:7. The platelets were centrifuged at 800 g for 10 min after each wash and were resuspended in Tyrodes solution (2 mM MgCl₂, 137 mM NaCl, 2.68 mM KCl, 3 mM NaH₂PO₄, 0.1% glucose, 5 mM Hepes and 0.35% albumin, pH 7.35) and count the number of cells.

1.2 Preparation of Platelet-Rich Plasma

Blood was collected into a tube containing 3.8% sodium citrate. Platelet-rich plasma was prepared by centrifugation at 250×g for 10 minutes.

### Example 2

### Platelet Aggregation

For platelet aggregation studies, platelet rich plasma (PRP) and washed platelets were stirred at 500 rpm at 37° C. in whole blood Lumiaggregometer (Chronolog, Havertown, Pa.). The difference in light transmission through the platelet suspension and suspending medium was taken as 100% aggregation. The effect of Y1 on platelet aggregation was evaluated by addition of different concentration of Y1 before the addition of agonist, and the effect was recorded for four minutes.

### Example 3

### Treatment of Platelets with Endoproteases

3.1 Cleavage of Platelets by Mocarhagin

For mocarhagin digestion, 10⁸ washed platelets in TS buffer ( 0.01 M Tris, 0.15 M sodium chloride, pH 7.4) containing 1 mM calcium chloride were treated with 12 μg/ml mocarhagin (final concentration) for 1 hour at 22° C. and digestion was stopped by adding EDTA to 0.01 M.

3.2 Cleavage of Glycocalicin by Cathepsin G

10⁸ washed platelets in TS buffer containing 1 mM calcium chloride was incubated with 1.8 μg/ml cathepsin G (final concentration) for 4 hours at 22° C. and digestion was stopped by adding PMSF to 1 mM.

3.3 Cleavage of Glycocalicin by O-Sialoglycoprotein Endoprotease

10⁶ platelets in 0.1 M Tris buffer pH 7.4 containing 0.2% albumin and protease inhibitors (10 μM leupeptin, 0.24 mM PMSF) was incubated with 0.14 mg/ml O-Sialoglycoprotein endoprotease (final concentration) for 45 min at 37° C. and digestion was stopped by adding sample buffer and boiling. The sample buffer used contained 3% SDS, 12% glycerol, 50 mM TrisHCl, 2% β-mercaptoethanol, and 0.03% bromphenol blue.

### Example 4

### Cleavage of Glycocalicin by Endoproteases

Cleavage of Glycocalicin by Mocarhagin

For mocarhagin digestion, glycocalicin in TS buffer containing 1 mM calcium chloride was incubated with 10 μg/ml mocarhagin (final concentration) for 1 hour at 22° C. and digestion was stopped by adding EDTA to 0.01 M.

Cleavage of Glycocalicin by Cathepsin G

For cathepsin G digestion, glycocalicin in TS buffer containing 1 mM calcium chloride was incubated with 3.4 μg/ml cathepsin G (final concentration) for 4 hour at 22° C. and digestion was stopped by adding PMSF to 1 mM.

Cleavage of Glycocalicin by O-Sialoglycoprotein Endoprotease

Glycocalicin in 0.05 M Tris buffer pH 7.4 was incubated with 1.2 mg/ml O-Sialoglycoprotein endoprotease (final concentration) for 15 min at 37° C. and digestion was stopped by adding sample buffer (as described in Example 3(YH) and boiling.

### Example 5

### Construction of Full Sized Y1 IgG

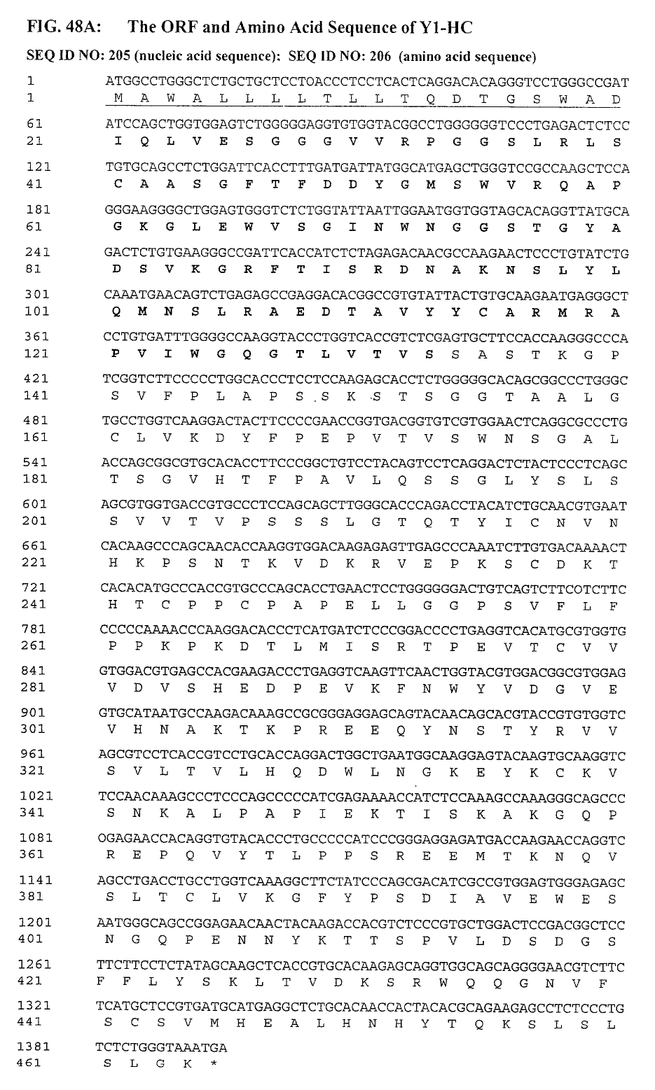

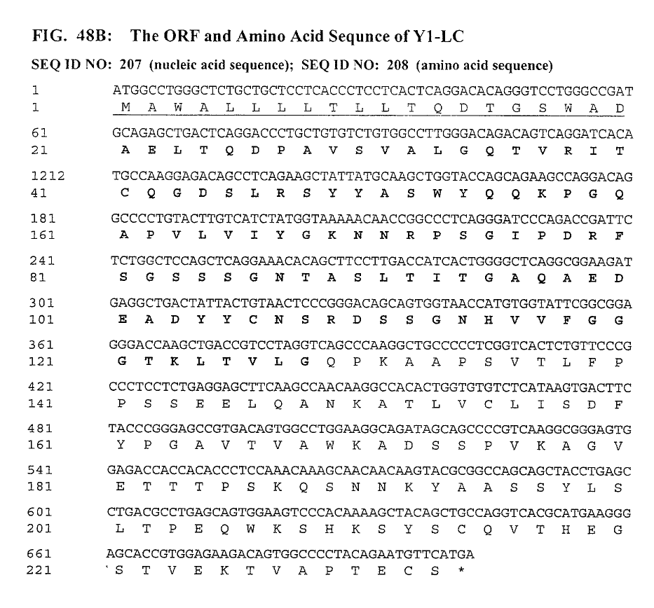

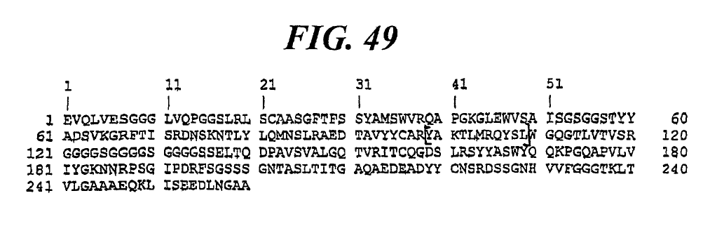

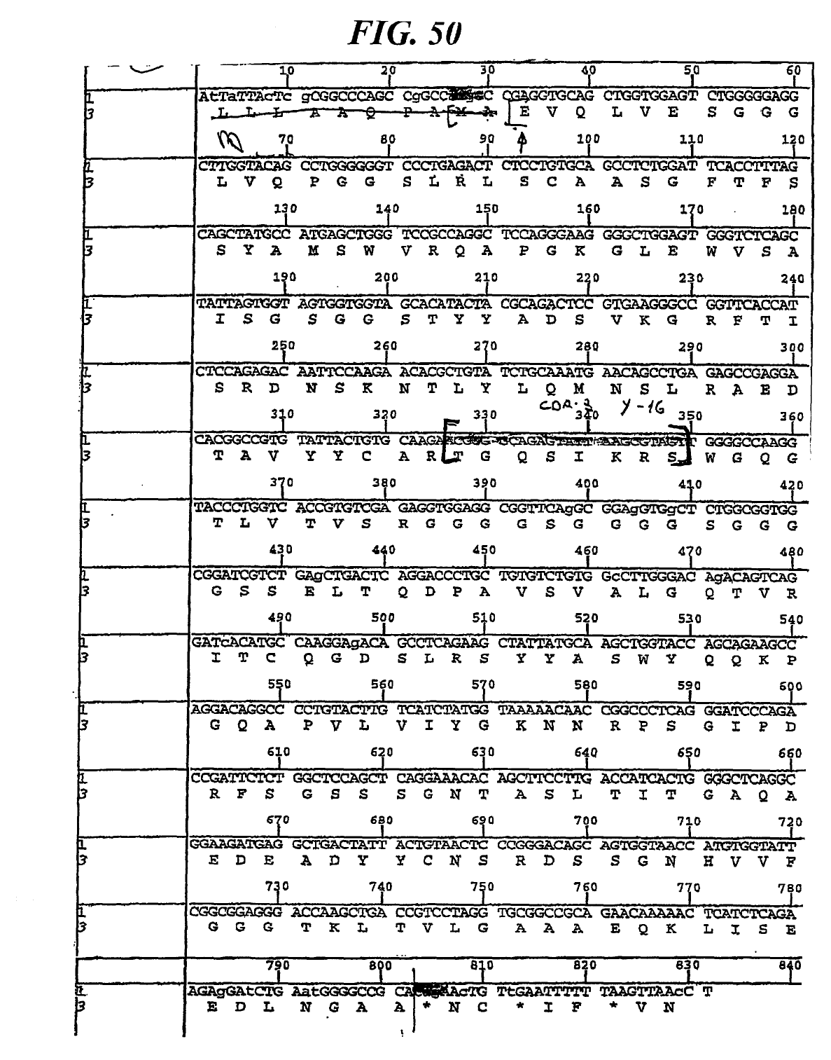

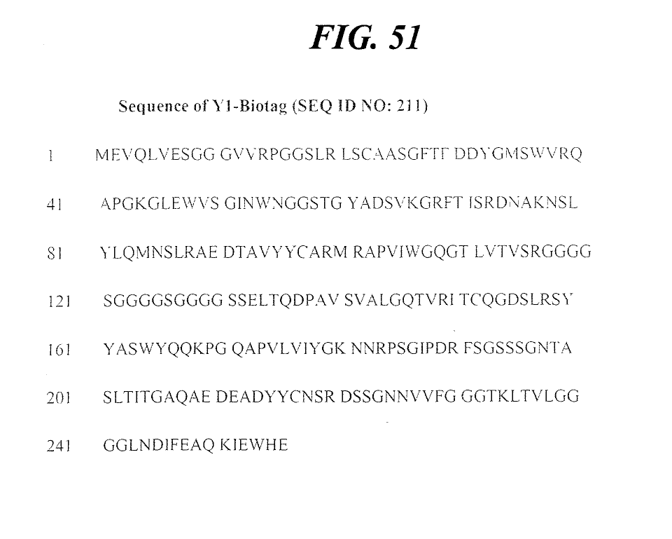

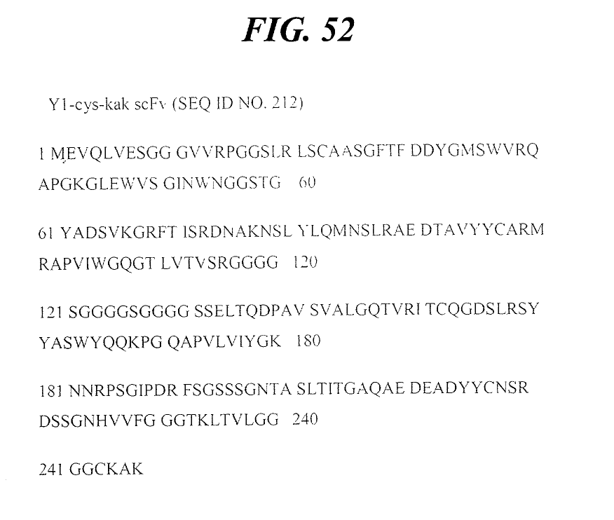

Whole IgG molecules have several advantages over the Fv forms, including a longer half-life in vivo and the potential for inducing an in-vivo cellular response, such as those mediated by ADCC or CDC (complement dependent cytotoxicity; Tomlinson, Current Opinions of Immunology, 5, 83-89(1993)). By a molecular cloning approach that is described below, we have converted the Y1 Fv regions into full sized IgGI molecules. Y1-IgG1 construction was accomplished by joining fragments of cDNA to each other in the following order: The sequence of the Y1-IgG heavy and light chains are presented in FIG. 48. The open reading frame (ORF) of the nucleotide sequence of Y1-HC (SEQ ID NO: 205), the amino acid sequence of Y1-HC (SEQ ID NO: 206), the ORF of the nucleotide sequence of Y1-LC (SEQ ID NO: 207), and the amino acid sequence of Y1-LC (SEQ ID NO: 208) are presented.

A leader sequence compatible for a mammalian expression system: An exchangeable system was designed to allow convenient insertion of elements required for a full IgG molecule. The following complimentary double stranded oligonucleotides encoding a putative leader sequence were synthesized, annealed, and ligated into the XhoI site of the pBJ-2 mammalian expression vector (under the SRα5 promoter).

5′-TCGACCTCATCACCATGGCCTGGGCTCTGCTGCTCCTCACCCTCCTCACTCA GGACACAGGGTCCTGGGCCGAT and

5′-GATCGATTGCACCAGCTGGATATCGGCCCAGGACCCTGTGTCCTGAGTGAG GAGGGTGAGGAGCAGCAGAGCCCAGGCCATGGTGATGAGG. Upstream of the initiation ATG codon, two Kozak elements were included. In addition, an internal EcoRV site was introduced between the putative cleavage site of the leader sequence and the XhoI site to allow subcloning of the variable regions. This modified vector was designated pBJ-3.

The V_{L} encoding sequence derived from the Y1 scFv cDNA sequence was inserted between the leader and the constant light region-encoding sequence. Similarly, the V_{H} encoding sequence derived from the Y1 scFv cDNA sequence was inserted between the leader and the constant heavy region-encoding sequence This was accomplished by PCR amplification of the vector pHEN-Y1, encoding for the original Y1, to obtain the V_{L} and the V_{H} regions, individually.

The Oligonucleotides

5′-TTTGATATCCAGCTGGTGGAGTCTGGGGGA (sense) and 5′-GCTGACCTAGGACGGTCAGCTTGGT (anti-sense) were used for the VL PCR reaction. The cDNA product of the expected size of 350 bp was purified, sequenced and digested with EcoRV and AvrII restriction enzymes. The same procedure was employed to amplify and purify the V_{H} cDNA region, using the sense and the anti-sense oligonucleotides

5′-GGGATATCCAGCTG(C/G)(A/T)GGAGTCGGGC and

5′-GGACTCGAGACGGTGACCAGGGTACCTTG, respectively.

Constant Regions:

The constant λ3 (CL-λ3) region and the constant heavy regions CH1-CH3 derived for IgG1 cDNA were individually synthesized as follows:

For the constant CL-λ3 region, RT-PCR was performed on mRNA extracted from a pool of normal peripheral B-cells (CD19+cells) in combination with the sense 5′-CCGTCCTAGGTCAGCCCAAGGCTGC and the anti-sense 5′-TTTGCGGCCGCTCATGAACATTCTGTAGGGGCCACTGT oligonucleotides. The PCR product of the expected size (˜400 bp) was purified, sequenced, and digested with AvrII and NotI restriction enzymes.

For the constant IgG1 regions (γ chain), a human B cell clone (CMV—clone #40), immortalized at BTG, was selected for PCR amplification. This clone was shown to secrete IgG1 against human CMV and was also shown to induce ADCC response in in-vitro assays. For the CH1-CH3 cDNA, oligonucleotides

5′-ACCGCTCGAGTGC(T/C)TCCACCAAGGGCCCATC(G/C)GTCTTC (sense) and

5′-TTTGCGGCCGCTCATTTACCC(A/G)GAGACAGGGAGAGGCT (anti-sense) were synthesized and used for PCR amplification. As described for the CL cDNA encoding sequence, the PCR product of expected size (˜1500 bp) was purified, sequenced, and digested with AvrII and NotI restriction enzymes.

For the final expression vectors, a triple ligation procedure was carried out using the EcoRV-NotI pre-digested pBJ-3 vector, EcoRV-AvrII variable cDNAs and AvrII-NotI constant regions. The final vectors for heavy chain and light chain expression were designated pBJ-Y1-HC and pBJ-Y1-LC, respectively.

An additional vector, pBJ-Y1-LP, was constructed based on the pBJ-Y1-LC to allow double selection based on the puromycin resistant gene (PAC). In this vector the neomycin-resistant gene of the pBJ-Y1-LC plasmid was replaced with a fragment of ˜1600 bp encoding for the PAC gene (from the pMCC-ZP vector).

The open reading frame (ORF) of both the Y1-HC and Y1-LC and their encoded amino acid sequences are presented as SEQ ID NOS. 205-208.

The leader sequence is underlined. The V_{H} and V_{L} regions are each encoded by amino acid sequences that are bolded, followed by either the IgG1 (for the heavy chain) or the λ3 (for the light chain) constant region sequences.

Expression of Y1 Heavy and Light Chain in CHO Cells.

Vectors pBJ-Y1-HC and pBJ-Y1-LC were used individually for the transfection and selection of stable cells expressing the heavy or light chains. Following selection on G418 and cell growth, the secreted protein in the supernatant was analyzed for IgG1 expression by the capture EIA assay and by Western blot analysis, as described below:

Capture EIA Assay:

The wells of 96 well-plates were pre-coated with mouse anti-human IgG1 Fc (Sigma). The supernatant from above was added to the wells, and the presence of heavy chain IgG1 was detected with biotinylated goat anti-γ chain specific antibody (Sigma), streptavidin-HRP and substrate. An ELISA plate reader monitored development of the color at A₄₀₅.

Western Blot Analysis:

The supernatant for the above cells was run on 12.5% SDS-PAGE. Expression of each chain was detected with (a) goat anti-human IgG-HRP (H+L; Sigma Cat #A8667) for heavy chain detection and (b) biotinylated goat anti-human λ3 chain (Southern Biotechnology Association, Cat #2070-08) for light chain detection.

Expression of both chains was confirmed by the above assays, and co-transfection was carried out to obtain full size Y1-IgG1.

Expression and Purification of IgG-Y1:

Cell Culture and Transfection:

CHO cells were cultivated in F-12 medium with 10% fetal calf serum and 40 μg/ml gentamicin at 37° C. in 5% CO₂ atmosphere. One day before transfection 0.8-1×10⁶ cells were seeded on 90 mm dishes. The cultures were co-transfected with 10 μg of light and heavy chains DNA by the FuGene (Roche) transfection reagent technique. After 2 days of growth in nonselective medium, the cells were cultured for 10-12 days in F-12 medium containing 550 μg/ml neomycin and 3 μg/ml puromycin. The cells were trypsinized and cloned by limiting dilution of 0.5 cell/well in Costar 96-well plates. Individual colonies were picked, grown in six-well dishes and transferred to flasks.

Determination of Heavy and Light Chain Secretion:

A sandwich ELISA assay was used to determine the concentration of the antibody secreted into the supernatant of transfected CHO cells. In order to determine the concentration of the antibody, the following reagents were used: monoclonal anti human IgG1(Fc) (Sigma) as the coated antibody, goat anti-human IgG (γ-chain specific) biotin conjugate as the detector (Sigma), and pure human IgG1, lambda (Sigma) as standard. Based on this ELISA assay the production rate varied between 3-4 μg/ml.

Production and Purification of MAb from the Cells:

Cells were grown in roller bottles to a final concentration of 1-2×10⁸ cells per bottle in F-12 medium with 10% fetal calf serum, supplemented with neomycin and puromycin. For the production, cells were cultured in the same medium, but with 2% of fetal calf serum for an additional two days.

The secreted antibody was purified on a protein G-Sepharose column (Pharmacia). Binding was in 20 mM sodium phosphate buffer, pH 7.0; elution was performed in 0.1M glycine buffer, pH 2.5-3.0. The quantity of the purified antibody was determined by UV absorbance; purity was analyzed by SDS-PAGE. Under non-denaturing conditions the full IgG antibody has its expected molecular weight of 160 kD. In denaturing gels both heavy and light chains have the expected molecular size of 55 and 28 kD, respectively.

Binding of Full Size IgG-Y1 Molecule:

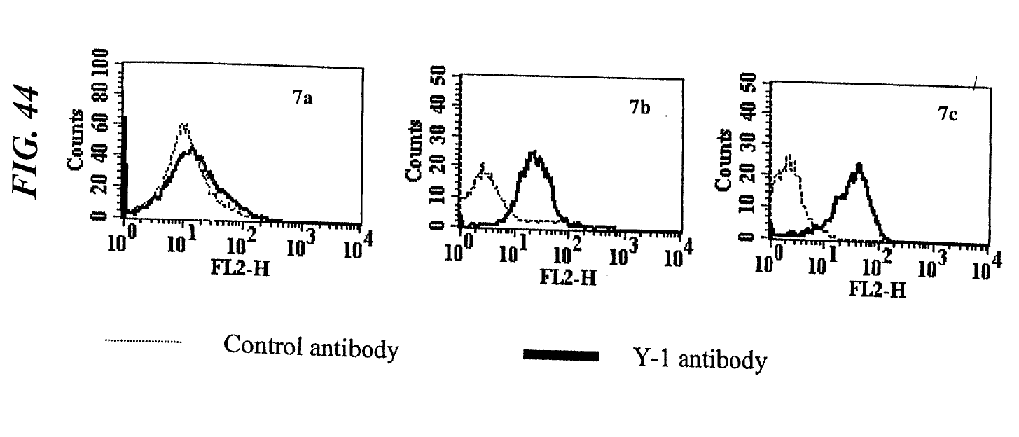

Binding experiments were performed to determine the level of binding of the IgG-Y1 molecule compared to the binding level of the scFv-Y1 molecule. A two-step staining procedure was employed, wherein 5 ng of IgG-Y1 were reacted with both RAJI cells (negative control, FIG. 44) and Jurkat cells (Y1 positive cells, FIG. 44). For detection, PE-labeled goat anti-human IgG was used. Similarly, 1 μg of scFv-Y1 was reacted with Jurkat cells (FIG. 44), and PE-labeled rabbit anti-scFv was used for detection. Results indicate that both IgG-Y1 and scFv-Y1 bind to Jurkat cells, with approximately 10³-fold more scFv-Y1 molecules needed to obtain a level of detection similar to that of the IgG-Y1.

### Example 6

### Preparation of Fab and F(ab′)₂ Fragments Derived from the Full IgG Y1 Antibody

Cell Culture and Transient Transfection:

CHO⁻ cells were cultivated in F-12 medium supplemented with 10% fetal calf serum and 40 μg/ml gentamicin at 37° C. in 5% CO₂ atmosphere. One day before transfection 1-1.5-1×10⁶ cells were seeded on 90 mm dishes. The cultures were co-transfected with 10 μg of DNA encoding the variable light and variable heavy chains of the Y1 antibody, each in a separate eukaryotic expression system. Transfection was carried out with the FuGene (Roche) transfection reagent technique.

After 2 days of growth in nonselective growth media, the cells were cultured for 10-12 days in F-12 medium containing 550 μg/ml neomycin and 3 μg/ml puromycin. The cells were trypsinized and cloned by limiting dilution of 0.5 cell/well in Costar 96-well plastic plates. Individual colonies were picked, grown in six-well dishes and transferred to flasks for further selection (to determine level of expression and antibody secretion to the growth media).

Cell Culture and Long-Term Transfection:

CHO⁻ cells were cultivated in F-12 medium supplemented with 10% fetal calf serum and 40 μg/ml gentamicin at 37° C. in 5% CO₂ atmosphere. One day before transfection 0.8-1×10⁶ cells were seeded on 90 mm dishes. The cultures were transfected with 10 μg of DNA encoding the variable light and variable heavy chains of the Y1 antibody cloned under the CMV (cytomegalovirus) promoter and the dhfr gene under the sv-40 promoter. Transfection was carried out using the FuGene (Roche) transfection reagent technique. After 2 days of growth in nonselective growth media, the cells were cultured in a media containing 100 nm-5 μM methotrexate (MTX) and dialyzed fetal calf serum in order to select for clones (after limiting dilution) that express increased levels of the full Y1 antibody.

Determination of Heavy and Light Chains Secretion:

A sandwich ELISA assay was established to determine the concentration of the antibody that is being secreted into the supernatant of transfected CHO cells. In order, to quantitate the concentration of the antibody, the following reagents were used: a monoclonal anti human IgG1(Fc) (Sigma) as the coated antibody, a goat anti human IgG(γ-chain specific) biotin conjugate as the detector (Sigma) and a purified human IgG1, lambda (Sigma) as standard.

Production and Purification of Mab from the Cells:

Cells were grown in roller bottles to a final concentration of 1-2×10⁸ cells per bottle in F-12 medium supplemented with 10% fetal calf serum, neomycin and puromycin (as indicated above). For antibody production, cells were cultured in the same medium, but with 2% of fetal calf serum for additional two days.

The secreted antibody was purified on a protein G sepharose column (Pharmacia) and ion exchange column-Q sepharose (Pharmacia). Binding was in 20 mM sodium phosphate buffer pH 7.0, while elution was in 0.1M glycine buffer pH 2.5-3.0. The quantity of the purified antibody was determined by UV absorbance and ELISA, while its purity was analyzed by SDS-PAGE and HPLC. Under non-denaturing conditions the full IgG antibody has its expected molecular weight of 160 kD. In denaturing gels both heavy and light chains have the expected molecular size of 55 and 28 kD respectively.

Fragmentation of Y1 IgG into Fab and F(ab′)₂:

The IgG molecule is composed of two identical light chains and two identical heavy chains. These chains are held together in folds (domains) by a combination of non-covalent interactions and covalent bonds (disulfide linkages). The light chain consists of one variable domain and one constant domain. The heavy chain consists of a variable domain (V_{H}) and three separate constant domains (CH 1, 2 and 3). The “hinge” region between constant heavy domain one (CH1) and constant heavy domain two (CH2) is readily accessible to proteolytic attack by enzymes. Cleavage at this point produces Fab or F(ab′)₂ fragments and the Fc portion. The Fab portion of the molecule retains the antigen binding capability of the molecule, but has low nonspecific binding. The Fab portion is best suited to those situations where the antigen binding capabilities without effector functions are desired.

In vivo Fab and F(ab′)₂ fragments are used as diagnostic and therapeutic agents. To make cancer chemotherapeutic agents tumor-specific instead of damaging to all cells, the agents can be linked to antibodies that bind to cell surface antigens of tumors. Using Fab or F(ab′)₂ fragments in place of intact IgG offers several advantages:

(1) The fragments can more easily cross capillaries and diffuse to tissue surfaces.

(2) Fragments not bound to conjugate will be cleared more rapidly than intact unbound IgG; and, therefore, more of the fragment-therapeutic agent will reach the target area.

An initial attempt to prepare monovalent and divalent antibody fragments has been done by using immobilized Ficin (Pierce). Ficin cleavage produces F(ab′)₂ fragments in the presence of 1 mM cysteine. Similarly, by increasing the concentration of cysteine activator in the digestion buffer to 10 mM, Fab fragments can be created from the original IgG.

After digestion, the fragments are purified on an immobilized Protein A column. The F(ab′)₂ and Fab fragments were concentrated using a microconcentrator with either a 10,000 or 30,000 Dalton molecular weight cutoff. Protein recovery was determined using absorbance at 280 nm. Fragment purity was determined using gel electrophoresis.

Detailed Procedure for the Preparation of the Fab Fragment

1 mg of purified Y1 antibody was applied to a 2 ml column of Immobilized Ficin in Digestion buffer in a concentration of 2 mg/ml of cysteine. HCL (for the preparation of F(ab′)₂ fragments) and 20 mg/ml (for the preparation of Fab fragments), at 37° C. for 5 hours (for Fab) and 20 hours (F(ab′)₂). Reaction was terminated by eluting the digest with 4 ml of Immunopure Binding buffer. Separation of Fab or F(ab′)₂ fragments from undigested IgG and Fc fragments was done by using Protein A column with Binding buffer. The Fab or F(ab′)₂ was contained in the flow through. By reading the absorbance at 280 nm, the peak fractions containing the fragments were pooled. Fragments were concentrated and dialyzed against PBS by using microconcentrator with 10,000 Dalton molecular weight cutoff. Protein recovery, purity and characterization were determined by using absorbance at 280 nm, gel electrophoresis and HPLC.

Cell Extract (Lysate) Preparation

2×10⁶ cells were harvested and centrifuged in microcentrifuge (1300 rpm, 4° C., 5 minutes). To wash, 0.5-1 ml PBS+pi was applied to the pellet and mixed gently. The mixture was centrifuged as before. Washing with 0.5-1 ml PBS+pi was repeated, and the mixture was centrifuged as above. The pelleted cells were resuspended in lysis buffer (200 μl/20×10⁶ cell pellet). The lysis buffer used was 50 mM Tris pH 7.4, 1 mm PMSF 1% NP-40, and 1 mM EDTA, although other suitable lysis buffers may be used. The suspension was incubated for about 60 minutes on ice, then centrifuged (3000 rpm, 4° C., 5 min). The supernatant was collected and divided into aliquots.

Preparation of a Crude Membrane Fraction and Extraction of Membrane Proteins

Twenty volumes of homogenization buffer was added to one volume of packed cells. The homogenization buffer used was 2% (w/v) Tween 20, 1 mM MgSO₄, 2 mM CaCl₂, 150 mM NaCl, and 25 mM Tris-HCl, pH 7.4. The following protease inhibitors were also added: 1 mM PMSF, 5 μg/ml Leupeptin, and 5 μg/ml Aprotonin. The cells were homogenized using three to five strokes in a Potter-Elvehjem homogenizer with a rotating Teflon pestle (Ultra-Torex). The sample was kept cold during homogenization, then stirred for 1 hour in an ice-bath. The sample was subjected to a few additional strokes in the homogenizer, then centrifuged at 3000 g for 30 min at 4° C. The supernatant was collected and centrifuged at 45000 g (19000 rpm rotor ss-34) for 1 hour at 4° C. The supernatant from the 45000 g centrifugation was discarded. A solution of 50 mM Tris 7.4, 1 mM EDTA, 1% NP-40 and protease inhibitors was added to the pellet, and the dissolved pellet was put it on ice for one hour.

Membrane Fraction Purification on HPLC column

An RPC column (Pharmacia Type PLRP-S 300 A) was used for membrane fraction purification. The buffers used were (A) 20 mM Tris 8.0 and (B) 60% propanol in DDW. The flow rate was 1 ml/minute, with the exception of the wash step during which the flow rate was 2 ml/min. The whole procedure was performed at ambient temperature.

The elution sample after immunoprecipitation (IP) of Jurkat or KG-1 membrane fraction was added with sample buffer (62 mM Tris pH 6.8, 10% Glycerol, 3% SDS, 720 mM mercaptoethanol) diluted with buffer A at a ratio of 1:4.

The sample was loaded on the column, and the flow-through liquid was collected. The column was washed with buffer A until the optic density of the flow declined to zero.

The proteins were eluted from the column according to the following program: 5 minutes with 80% A, then 40 minutes gradient of 80-0% of A and 20-100% B. A second gradient was then applied to bring the composition of the flow to 80% A, and this composition was used to wash the column for 5 more minutes. The elution liquid was collected in a fraction collector, in 1 ml fraction sizes.

The samples were evaporated to small volumes in a Speed-Vac evaporator, then analyzed using SDS-PAGE (SDS-polyacrylamide gel electrophoresis) and western blotting.

Purification of Normal Human Plasma on Q Sepharose Columns

5 ml of normal human fresh frozen plasma was diluted 1:10 with start buffer and filtered through a 0.45 μm syringe filter (Sartorius, cat #16555). Start buffer is 20 mM Tris-HCl, pH 8.0 and contains protease inhibitors (5 μg/ml leupeptin, 5 μg/ml aprotinin, and 1 mM PMSF).

A 5 ml HiTrap Q Sepharose column (Amersham Pharmacia, cat #17-1154-01) was attached to a P-1 peristaltic pump (Amersham Pharmacia). The column was washed according to the commercial protocol at a flow rate of approximately 4 ml/minute. The diluted filtered plasma was loaded on the column, and the flow-through liquid was collected. The column was washed with 20 volumes of 0.3 M NaCl solution in start buffer. Proteins were eluted at 0.6 M, 0.8 M, and 1.0 M NaCl solutions in start buffer. Elution volumes were 50, 20 and 20 ml, respectively. The whole procedure was performed at 4° C.

Eluted fractions were subjected to SDS-PAGE analysis in duplicated gels, followed by Western blot analysis using biotinylated Y1 and antibody 181 as a negative control. Fibrinogen y prime was found in the 0.6M NaCl elution fraction. The 1.0 M NaCl elution fraction contained complement compound 4 (CC4), lumican, prothrombin, and inter-alpha-inhibitor.

Purification of Normal Human Plasma on HPLC Column

Q Sepharose purified normal human plasma was mixed with Urea (to a final concentration of at least 8M), DTT (to a final concentration of 30 mM) and TFA (to a final concentration of 0.1%).

The purified plasma solution was loaded on a 3 ml RPC column (Amers0ham Pharmacia), and the flow-through liquid was collected. The column was washed with buffer A until the optic density of the flow declined to zero. The proteins were eluted from the column according to the following program: 5 minutes with 90% A, then 40 minutes gradient of 90-0% of A and 10-100% B. A second gradient was then used to bring the composition of the flow to 90% A, and this composition was used to continue washing the column for 5 more minutes. The buffers used were (A) 0.1% TFA in DDW and (B) 80% CAN and 0.1% TFA in DDW. The flow rate was 1 ml/minute, with the exception of the wash step, during which the flow rate was 2 ml/minute.

The elution liquid was collected in a fraction collector, in 1 ml fraction sizes. The whole procedure was performed at ambient temperature.

The samples were evaporated to small volumes in a Speed-Vac evaporator, then analyzed using SDS-PAGE and western blot.

Indirect Immunoblotting with Non-Labeled CD162 Antibody

Samples were run on 10% SDS-PAGE at 140-160 Volts for 3.5 hours at Sigma Z37, 503-9 appliance. The electrophoresed samples were transferred onto a nitrocellulose membrane overnight on 20 Volts in Tris Glycine buffer (20% MeOH, 192 mM glycine, 25 mM TRIS, pH 8.3) at room temperature. The nitrocellulose membrane was blocked using 5% skim milk in PBS (phosphate buffered saline) for one hour at room temperature. The nitrocellulose membrane was washed 3 times for 5 minutes each with 0.05% Tween 20 in PBS. The membrane was incubated with Super Signal mixture (Pierce) for 5 minutes as directed in the commercial protocol, then excess solution was dried. The membrane was use to expose it to X-ray film (Fuji), and the film was developed.

Western Blot Analysis of Y1 Receptor—Processing of the Filter After Blotting

The nitrocellulose membrane was blocked using 5% skim milk for one hour at room temperature. The membrane was then washed 3 times for 5 minutes each with 0.05% Tween 20 in PBS at room temperature. The membrane was incubated with 5 μg/ml Y1-biotin (or 181-biotin) in 2% skim milk in PBS for one hour at room temperature. The membrane was then washed 3 times for 5 minutes each with cold 0.05% Tween 20 in PBS in the cold room (about 4 to about 10° C.). The membrane was then incubated in the cold room with a 1:1000 dilution of SAV-HRP (streptavidin-HRP) (at a final concentration of 1 g/ml) in 2% skim milk, 0.05% Tween. The dilution was carried out at room temperature (about 25° C.), then the diluted SAV-HRP was cooled on ice for 10-15 minutes before use. The incubation was carried out for 1 hour with gentle shaking. After the incubation with SAV-HRP, the membrane was washed, as above. The membrane was then incubated with Super Signal mixture (Pierce) for 5 minutes as directed in the commercial protocol, then excess solution was dried. The membrane was use to expose it to X-ray film (Fuji), and the film was developed.

### Example 7

### Prokaryotic Expression of Recombinant Glycocalicin (GC)

The DNA fragment encoding for glycocalicin (GC, amino acid 1 to amino acid 493 of GPIbα) was cloned into an IPTG inducible prokaryotic vector cassette. E. coli (BL21 DE3) cells harboring the newly constructed plasmid were grown at 37° C. to O.D. 0.7-0.8, than at 37° C. for 3 hours for in the presence of IPTG for induction.

SDS-polyacrylamide gel loaded either with semi-purified human platelet derived glycocalicin (“GC”) or with E. coli cell lysates (“total”) derived from induced and non-induced cells were analyzed. Western blot analysis was performed with biotinylated Y1-scFv, polyclonal rabbit anti-human glycocalicin antibody, commercially available mouse anti-human CD42 monoclonal antibody (SZ2 Immunotech, PM640 Serotec, HIP1 Pharmigen, AN51 DAKO), and polyclonal antibody against the N-terminus of GPIbα (Sc-7071, Santa Cruz).

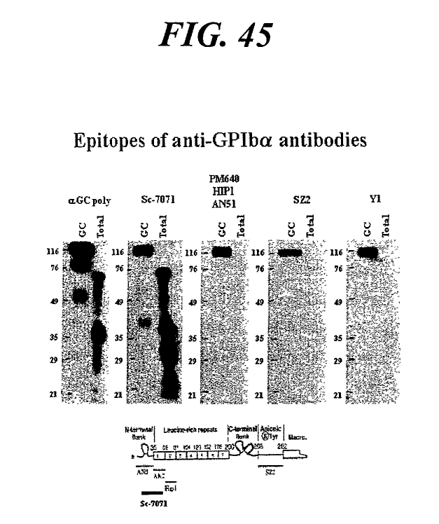

The two polyclonal antibodies recognized both the recombinant bacterial derived glycocalicin and the natural human platelet derived glycocalicin. The Y1-scFv and the commercially available antibodies recognized the natural human-derived glycocalicin, but did not recognize the bacterial derived recombinant platelet glycocalicin. FIG. 45.

The prokaryotic (e.g., E. coli) system lacks post-translational modification mechanisms, such as mechanisms for glycosylation and sulfation. Thus, the lack of recognition by Y1-scFv of the bacterially produced glycocalicin supports the conclusion that post-translational modification, such as glycosylation and sulfation, is essential for Y1-scFv binding to glycocalicin.

FACS Protocol for Blood/Bone Marrow Samples

Samples provided from hospitals. Patients sample 30 μl/tube. Add 5 μl /tube of CD33-APC (for AML) or CD19-APC (for B-CLL) or CD38-APC (for Multiple Myeloma). Add 5 μl/tube of CD45-PerCp and 5 μl of scFv-Y1 or control scFv-N31 or CD162-PE (KPL1). Incubate tubes 30 minutes at 4° C. with low shaking. Wash by adding 2 ml PBS and spin 5 minutes at 1200 rpm. Discard supernatant.

For a one step assay continue with the lysis step:

Add 500 μl BD Lysine solution diluted 1:10 with ddH₂O (300 μl to patient sample). Vortex at high speed and incubate 12 minutes at 4° C. Wash as above. Discard supernatant. And add 500 μl PBS. The samples are read in the FACS using blood sample acquisition setup according to international standards.

For two or more assays: working buffer is PBS+1% BSA+0.05% sodium azide. Incubations and wash as above.

Lysis of the red blood cells is the final step in the assay, followed by resuspension with 500 μl PBS.

### Example 8

### Construction of Triabodies

The vector pHEN-Y1, encoding the original Y1, was amplified using PCR for both the V_{L} and the V_{H} regions, individually. The sense oligonucleotide 5′-AACTCGAGTGAGCTGACACAGGACCCT, and the anti-sense oligonucleotide 5′-TTTGTCGACTCATTTCTTTTTTGCGGCCGCACC were used for the VL PCR reaction. The cDNA product of the expected size of ˜350 bp was purified, sequenced, and digested with XhoI and NotI restriction enzymes.

The same procedure was employed to amplify the V_{H} region (using the sense oligonucleotide 5′-ATGAAATACCTATTGCCTACGG and anti-sense oligonucleotide 5′-AACTCGAGACGGTGACCAGGGTACC). The V_{H} PCR product was digested with NcoI and XhoI restriction enzymes. A triple ligation procedure into the pHEN vector, pre-digested with NcoI-NotI, was employed. The final vector was designated pTria-Y1.

Following E. coli transformation, several clones were picked for further analysis, which included DNA sequencing, protein expression, and extraction from the periplasmic space of the bacteria. SDS-PAGE under reducing conditions and Western blot analysis were performed to confirm the size of the Y1 triabodies.

### Example 9

### Construction of Diabodies

The pTria-Y1 vector from above was linearized with XhoI restriction enzyme, and synthetic complimentary double stranded oligonucleotides (5′-TCGAGAGGTGGAGGCGGT and 5′TCGAACCGCCTCCACCTC) were pre-annealed and ligated into the XhoI site, between the Y1-heavy and Y1-light chains. This new vector was designated pDia-Y1. As described for the triabodies, the DNA sequence and protein expression was confirmed.

### Example 10

### Expression and Purification of Diabodies and Triabodies

Expression in E-coli was essentially as described for the scFv-Y1. However, the purification of Y1 diabodies and triabodies from the periplasm of the transformed E. coli cells was different. The scFv Y1 monomer form can be purified on an affinity column of Protein-A Sepharose beads. Multimeric forms of Y1 are, however, ineffectually purified by this procedure. Therefore, periplasmic proteins extracted from the bacteria were precipitated over-night with 60% ammonium sulfate, resuspended in H₂0, and loaded onto a Sephacryl-200 (Pharmacia) size exclusion column pre-equilibrated with 0.1.×PBS. Fractions were collected and analyzed by HPLC, and separate fractions containing either the dimer or timer forms were collected for FITC labeling and FACS analysis.

### Example 11

### Binding of Y1 Diabodies and Triabodies to Cells

FACS analysis was performed on Jurkat cells using a “three step staining procedure.” First, crude extracts or purified unlabeled scFv are stained, then mouse anti-myc antibodies, and finally, FITC- or PE-conjugated anti-mouse antibodies. FACS analysis requires 5-8×10⁵ cells, which have been Ficoll-purified and resuspended in PBS+1% BSA. Binding was carried out for 1 hour at 4° C. After each step, cells were washed and resuspended in PBS+1% BSA. After the final staining step, cells were fixed by re-suspending in PBS, 1% BSA, 2% formaldehyde, and then read by FACS (Becton-Dickinson).

The binding of Y1-scFv was compared to that of diabodies and triabodies. In this analysis (FIG. 44), the binding profile of all three forms was very similar, indicating that the above modifications in the molecule did not alter, conceal or destroy the apparent binding affinity of Y1 to its ligand.

### Example 12

### A Study of the Affinity of the S-S Y1-Dimer in Comparison to CONY1 and Y1-IgG, using a Radioreceptor Binding Assay with KG-1 Cells

The assay system involved the use of radioactive ligands that were prepared by iodination with ¹²⁵I using chloramine T on the Y1-IgG construct or the Bolton-Hunter (CONY1) reagent. The assay tubes contained 5×10⁶ KG-1 cells per 0.2 ml and a labeled tracer with varying amounts of unlabeled competitor, in PBS+0.1% BSA, pH 7.4. After one hour incubation with agitation at 4° C., the cells were thoroughly washed with cold buffer and taken for radioactivity counting.

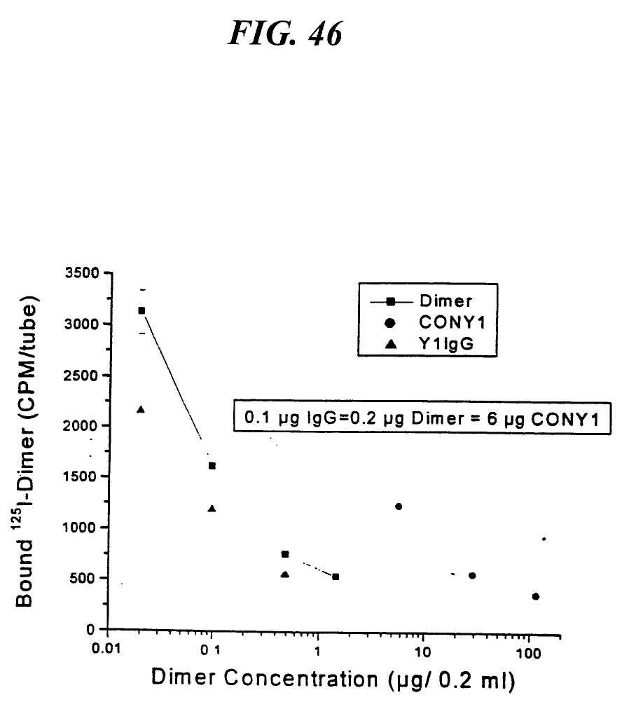

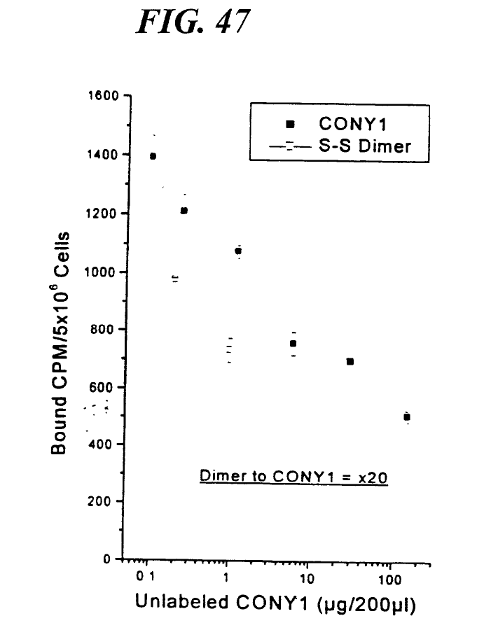

For the radio receptor binding assay (RRA), 2 ng/tube of ¹²⁵I-Y1-IgG was used, and competition was performed with each of the three molecules. The results are provided in FIG. 46. The results presented in this figure demonstrate that the affinity of the S-S Y1 dimer was twofold lower than most of the full Y1 antibody and 30 times higher than that of CONY 1. A rough estimate of the affinity of the Y1-IgG in this experiment is 2×10⁻⁸ M. The corresponding affinity of the dimer is, therefore, 4×10⁻⁸ M.

In a second RRA using labeled CONY1, a 100 ng/tube of ¹²⁵I-Y1-IgG was used, and competition was performed with each of the three molecules. The results are provided in FIG. 47. This figure shows that the affinity of the S-S dimer was 20 times higher than that of CONY1. A rough estimate of the affinity of CONY 1 in this experiment is 10⁻⁶M. The corresponding affinity of the dimer is, therefore, 5×10⁻⁸ M.

### Example 13

### Production of Y1-cys-kak (Cysteine Dimer)

One liter of λpL-y1-cys-kak bacterial culture was induced at 42° C. for 2-3 hrs. This culture was centrifuged at 5000 RPM for 30 minutes. The pellet was resuspended in 180 ml of TE (50 mM Tris-HCl pH 7.4, 20 mM EDTA). 8 ml of lysozyme (from a 5 mg/ml stock) was added and incubated for 1 hr. 20 ml of 5M NaCl and 25 ml of 25% Triton was added and incubated for another hour. This mixture was centrifuged at 13000 RPM for 60 minutes at 4° C. The supernatant was discarded. The pellet was resuspended in TE with the aid of a tissuemiser (or homogenizer). This process was repeated 3-4 times until the inclusion bodies (pellet) were gray/light brown in color. The inclusion bodies were solubilized in 6M Guanidine-HCl, 0.1 M Tris pH 7.4, 2 mM EDTA (1.5 grams of inclusion bodies in 10 ml solubilization buffer provided ˜10 mg/ml soluble protein). This was incubated for at least 4 hrs. The protein concentration was measured and brought to a concentration of 10 mg/ml. DTE was added to a final concentration of 65 mM and incubated overnight at room temperature. Re-folding was initiated by dilution of 10 ml of protein (drop by drop) to a solution containing 0.5 M Arginine, 0.1 M Tris pH 8, 2 mM EDTA, 0.9 mM GSSG. The re-folding solution was incubated at ˜10° C. for 48 hrs. The re-folding solution containing the protein was dialyzed in a buffer containing 25 mM Phosphate buffer pH 6, 100 mM Urea, and concentrated to 500 ml. The concentrated/dialyzed solution was bound to an SP-sepharose column, and the protein was eluted by a gradient of NaCl (up to 1M).

### Example 14

### ELISA to GC (Glycocalicin)

100 ml of purified glycocalicin was incubated in a 96 flat well maxisorp plates, overnight at 4 degrees celsius. The plate was washed with PBST (PBS+0.05% tween) 3 times, then 200 ml of PBST-milk (PBST+2% Non fat milk), for 1 hr at room temperature. The plate was washed with PBST, and the monomer or dimer (100 ml) was added in PBST-milk at different concentrations for 1 hr at room temp. Then the plate was washed and anti-V_{L} polyclonal (derived from immunized rabbits with V_{L} derived from Y1) (1:100 diluted in PBST-milk) was added for an hour. The plate was washed and anti-rabbit HRP was added for an additional hour. The plate was washed 5 times and 100 μl TMB substrate was added for approximately 15 minutes then 100 μl of 0.5 H₂SO₄ was added to stop the reaction. The optical density of the plate was measured at 450 nm in an ELISA reader.

### Example 15

### E. coli Expression of Recombinant Glycocalicin (GC)

The DNA fragment encoding the N-terminal soluble part of human platelet GP1b-glycocalicin (GC, amino acid 1 to amino acid 493) was cloned into an IPTG inducible prokaryotic vector cassette. E. coli (BL21 DE3) cells harboring the newly constructed plasmid were grown at 37° C. to O.D. 0.7-0.8, than at 37° C. for 3 hours for in the presence of IPTG for induction. SDS-polyacrylamide gel loaded either with semi-purified human platelet derived GC or with E. coli cell lysates (total protein content) derived from induced and non-induced cells were analyzed. Western blot analysis was performed with scFv Y1-biotinylated, polyclonal rabbit anti-human GC antibody, mouse anti-human CD42 monoclonal antibody (SZ2 Immunotech, PM640 Serotec, HIP1 Pharmigen, AN51 DAKO, commercially available) and polyclonal antibody against the N-terminus of GPIba (Sc-7071, Santa Cruz). The two polyclonal antibodies recognized both the recombinant bacterial derived GC as well as the natural human platelet derived GC. The scFv Y1 and the commercially available antibodies recognized only the natural human derived GC, but not the bacterial derived recombinant platelet GC.

Post-translational modification, such as glycosylation and sulfation is essential for scFv and commercially available antibodies binding to GC. The prokaryotic (E. coli) system lacks post-translation modification mechanisms, such as glycosylation and sulfation.

### Example 16

### Production of Tetramers of Y1

A construct was designed where the following sequence, LNDIFEAQKIEWHE, was added at the C-terminus of the Y1 by PCR and cloning into an IPTG inducible expression vector cassette. The clone was named Y1-biotag. This sequence is a substrate for the enzyme BirA, that in the presence of free biotin, the enzyme is capable of covalently connecting biotin to the lysine (K) residue (Phenotypic analysis of antigen-specific T lymphocytes. Science. Oct. 4, 1996; 274(5284):94-6, Altman J D et al). This construct was produced as inclusion bodies in BL21 bacterial cells. Refolding was performed as described previously. Inclusion bodies were solubilized in quanidine-DTE. Refolding was done by dilution in a buffer containing arginine-tris-EDTA. Dialysis and concentration was performed followed by HiTrapQ ionic exchange purification.

The purified Y1-biotag scFv was incubated with BirA enzyme (purchased from Avidity) and biotin as recommended by the provider. The biotinylated Y1-biotag was analyzed by HABA test (that estimates the amount of biotin per molecule) and demonstrated that there was around >0.8 biotin residues/molecule.

The Y1-biotag biotinylated was incubated with Streptavidin-PE (Phycoerythrin) to form complexes and used in FACS experiments using KG-1 cells (positive for Y1). The sensitivity of the binding was increased at least 100 fold due to the increase in avidity. Streptavidin can bind up to 4 biotinilated-Y1-biotag molecules.

The sequence of Y1-biotag is as follows, and is SEQ ID NO: 211:

| | |
|---|---|
| 1 | MEVQLVESGG GVVRPGGSLR LSCAASGFTF DDYGMSWVRQ |
| 41 | APGKGLEWVS GINWNGGSTG YADSVKGRFT ISRDNAKNSL |
| 81 | YLQMNSLRAE DTAVYYCARM RAPVIWGQGT LVTVSRGGGG |
| 121 | SGGGGSGGGG SSELTQDPAV SVALGQTVRI TCQGDSLRSY |
| 161 | YASWYQQKPG QAPVLVIYGK NNRPSGTPDR FSGSSSGNTA |
| 201 | SLTITGAQAE DEADYYCNSR DSSGNNVVFG GGTKLTVLGG |
| 241 | GGLNDIFEAQ KIEWHE |

### Example 17

### Characterization of Y17 Activity

The enzyme O-Sialoglycoprotein endoprotease from Pastoral haemolytica that selectively cleaves human platelets GPIb, was used in order to establish the specificity of binding of Y17 to GPIbα. The O-Sialoglycoprotein endoprotease, specifically cleaved only proteins containing sialyated, O-linked glycans, and does not cleave N-linked glycoproteins or unglycosylated proteins. This enzyme has been reported to cleave GPIb, which is heavily O-glycosylated, but not GPIIb-IIIa or other receptors on the platelets. Incubation of washed platelets with O-Sialoglycoprotein endoprotease which cleaved GPIb, abolishes binding of Y17 as well as the binding of monoclonal antibody (MCA466S-serotec) directed against GPIbα to the GPIb as was shown by immunoblots and by FACS analysis. These endoprotease did not change the binding of monoclonal antibody (anti CD61) directed against GPIIb/IIIa (FIG. 4).

### Example 18

### Identification of Y17 Epitope on Platelets GPIb

An interesting approach to identification of the Y1 epitope on platelets GPIb is to use endoproteases enzyme whose cleavage sites on platelets GPIb are fully characterized.

18.1: Effect of Mocarhagin on the Mapping of Y1 Epitope

Mocarhagin, a cobra venom metalloproteinase cleaves platelet GPIbα specifically at a single site between residues glu-282 and asp-283, generates two stable products, a 45-kDa N-terminal fragment (His-1-Glu-282) found in the supernatant and a membrane bound 100 kDa C-terminal fragment.

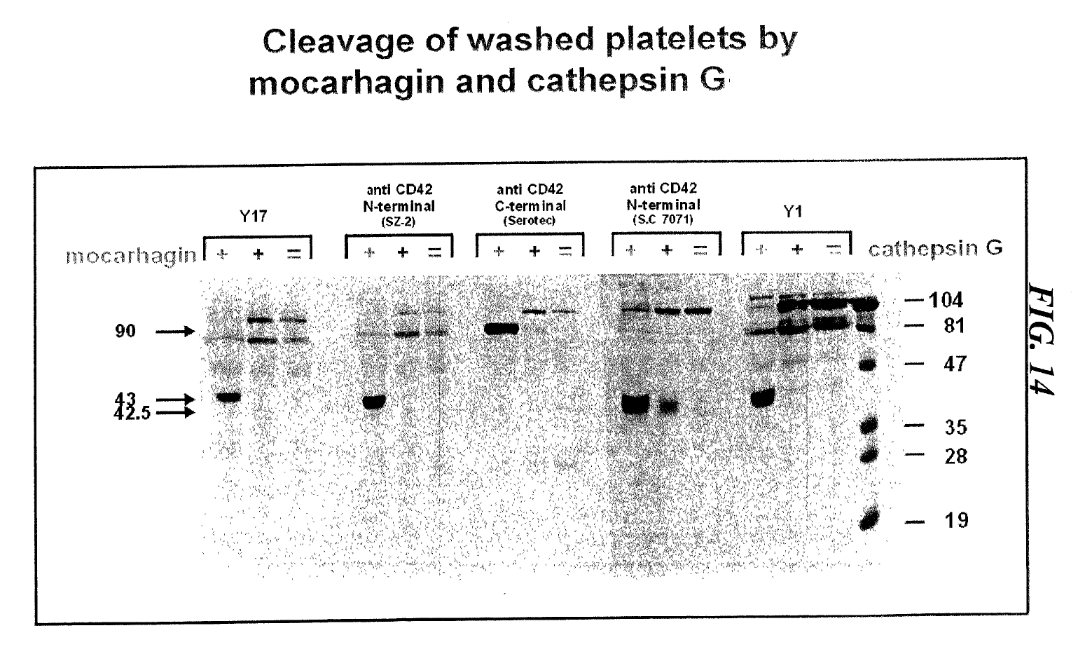

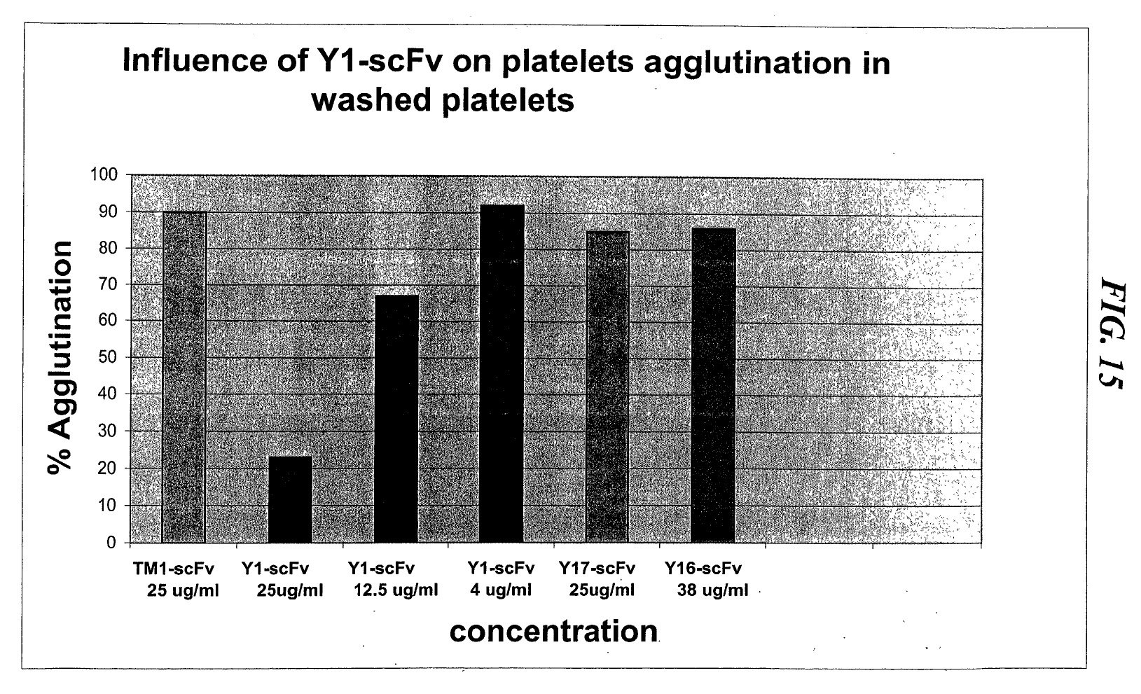

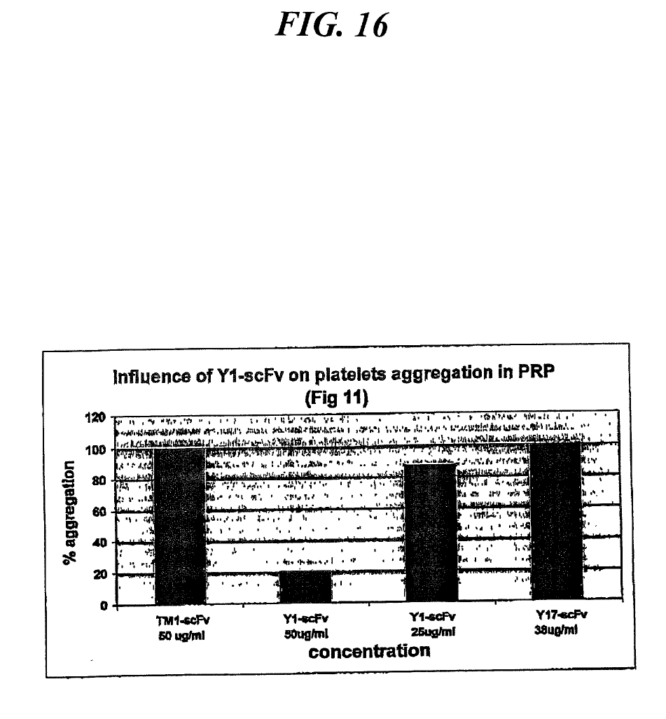

Washed platelets were treated by mocarhagin and, platelets lysate were separated on SDS-polyacrylamide gels and transferred to nitrocellulose. Analysis of mocarhagin-treated washed platelets by Western blot analysis with Y1-results in loss of the band corresponding to GPIb (135 kDa) and, binding of Y17 to the N-terminal 45 kDa tryptic fragment. Monoclonal antibodies, MCA466S directed against the C-terminal fragment of GPIbα reacted with the 100 kDa C-terminal fragment while, monoclonal antibody S.C.7071 which recognizes the N-terminal of GPIbα reacted with the same 45 kDa N-terminal fragment that was recognized by Y17 (FIG. 14).

Mocarhagin treatment of glycocalicin gave results similar to those observed with washed platelets, showing binding of Y1 and monoclonal antibodies, S.C. 7071 to 45 kDa N-terminal cleavage product fragment of GPIbα (FIG. 8). The results suggest that the epitope for Y17 is contained within the sequence His-1-Glu-282.

18.2: Effect of Cathepsin G on the Mapping of Y17 Epitope

Cathepsin G, a neutrophil serine protease, cleaved glycocalicin between residues leu-275 and Tyr-276 and a second cleavage site between residues Val-296 and Lys-297. Glycocalicin treated by cathepsin G generated two N-terminal fragments, a small fragment 42 kDa fragment (His1-Leu275) and a large 45 kDa N-terminal fragment (His1-Val-296), in addition to a ˜95 kDa C-terminal fragment. Glycocalicin and glycocalicin fragments generated by cathepsin G digestion were separated on SDS-polyacrylamide gels and transferred to nitrocellulose. Y17 bound to the larger fragment (His1-Val-296), but not to the smaller fragment (His1-Leu275). Moreover, monoclonal antibody S.C. 7071 which recognizes an epitope within His1-Leu275 blotted both fragments (FIG. 12). Analysis of N-terminal peptide proteolytic fragments of mocarhagin and cathepsin G suggests that the BPIbα amino acid sequence Tyr-276-Glu-282 is an important recognition motif for binding of Y17.

### Example 19

### Effect of Y17-scFv on vWF-Dependent Agglutination

The effect of Y17-scFv on vWF-dependent agglutination of platelets was tested at different concentrations of Y17. In contrast to Y1, Y17 at a final concentration of 10, 25 or 50 μg/ml did not inhibit vWF-dependent platelet agglutination in washed platelets induced by ristocetin. Analysis of N-terminal peptide proteolytic fragments of mocarhagin and cathepsin G suggests that the GPIbα amino acid sequence Tyr-276-Glu-282 is an important recognition motif for binding of Y17 and Y1. Since Y17 does not inhibit platelet aggregation, it seems that Y1 and Y17 do not bind to the same sequences, but to overlapping sequences.

We claim:

1. An antibody multimer comprising at least a first and a second antigen binding fragment, wherein the at least first or second antigen binding fragment or both is capable of binding or cross-reacting with an epitope comprising the formula 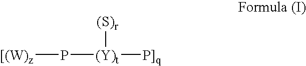
Wherein:
W is any amino acid other than Aspartate and Glutamate
Y is any naturally occurring moiety that is capable of being sulfated
P is (A)ₘ(A)ₙ(X)ᵤ or (X)ᵤ(A)ₙ(A)ₘ or (A)ₙ(X)ᵤ(A)ₘ or (A)ₙ(A)ₘ(X)ᵤ or (X)ᵤ(A)ₘ(A)ₙ or (A)ₘ(X)ᵤ(A)ₙ
S is sulfate or a sulfated molecule
X is any amino acid except Aspartate, Glutamate, or Tyrosine
A is any negatively charged amino acid or leucine, isoleucine, proline, phenylalanine, serine, or glycine
q is 1 to 6
z is 0, 1, or 2
r is 0 or 1
t is 1, 2 or 3
u is 0 to 2
n is 0 to 3
m is 0to 3
wherein if n=0 then m>0; wherein if m=0 then n>0; wherein if q is 1, r is 1, and if q is >1 at least one of Y is sulfated.

2. An antibody multimer of claim 1 wherein the first or second antigen binding fragment or both binds or cross reacts with the epitope in which:
W is Glycine,
Y is a peptido conjugate of Tyrosine or a glyco conjugate of sparagine, Serine or Threonine.
A is Glutamate, y Carboxy Glutamate or Aspartate
q is 1, 2, or 3.

3. An antibody multimer of claim 1 wherein the first or second antigen binding fragment or both binds or cross reacts with the epitope in which:
Y is a peptido conjugate of Tyrosine
q is 3
r is 1.

4. An antibody multimer comprising at least a first and second antigen binding fragment, wherein the first or second antigen binding fragment or both is capable of binding or cross-reacting with an epitope comprising the formula 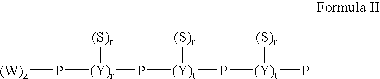
Wherein:
W is any amino acid other than Aspartate and Glutamate
Y is any naturally occurring moiety that is capable of being sulfated
P is (A)ₘ(A)ₙ(X)ᵤ or (X)ᵤ(A)ₙ(A)ₘ or (A)ₙ(X)ᵤ(A)ₘ or (A)ₙ(A)ₘ(X)ᵤ or (X)ᵤ(A)ₘ(A)ₙ or (A)ₘ(X)ᵤ(A)ₙ
S is a sulfate or a sulfated molecule
X is any amino acid except Aspartate, Glutamate or Tyrosine
A is any negatively charged amino acid or leucine, isoleucine, proline, phenylalanine, serine, or glycine
z is 0, 1, or 2
r is 0 or 1
t is 1, 2 or 3
u is 0to 2
n is 0to 3
m is 0 to 3
wherein if n=0 then m>0; wherein if m=0 then n>0; wherein at least one Y is sulfated.

5. An antibody multimer of claim 4 wherein the first or second antigen binding fragment or both binds or cross reacts with the epitope in which:
W is Glycine
Y is a peptide conjugate of Tyrosine or a glyco conjugate of Asparagine, Serine or Threonine
A is Glutamate, y Carboxy Glutamate or Aspartate, Leucine, Isoleucine, Proline, Phenylalanine, Serine, or Glycine.

6. An antibody multimer of claim 4 wherein the first or second antigen binding fragment or both binds or cross reacts with the epitope in which:
Y is a peptido conjugate of Tyrosine
q is 3; and
r is 1.

7. An antibody multimer comprising at least a first and second antigen binding fragment, wherein the at least first or second antigen binding fragment or both is capable of binding or cross-reacting with an epitope comprising the formula 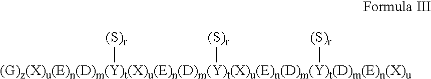
Wherein:
G is Glycine
E is Glutamate
D is Aspartate
Y is Tyrosine
S is sulfate or a sulfated molecule
X is any amino acid except the above
z is 0, 1, or 2
t is 1, 2or 3
r is 0 or 1
u is 0 to 2
n is 0 to 3
m is 0 to 3
wherein at least one Y is sulfated; wherein if n=0 then m>0; wherein if m=0 then n>0.

8. An antibody multimer of claim 7 wherein the first or second antigen binding fragment or both binds or cross reacts with the epitope in which r is 1.

9. An antibody multimer of claim 1, 4 or 7 wherein the multimer is a dimer, trimer or tetramer.

10. An antibody multimer of claim 9 wherein the multimer is a dimer.

11. A dimer of claim 10 wherein at least one of the first and second antigen binding fragments is selected from scFv fragments of Y1 and Y17.

12. A dimer of claim 10 wherein the first and second antigen binding fragments are linked by a disulfide bridge.

13. A dimer of claim 12 wherein the first and second antigen binding fragments are Y1-CysKAK.

14. A dimer of claim 10 wherein the first and second antigen binding fragments are linked by a polypeptide linker of 5 to 20 amino acids.

15. A dimer of claim 14 wherein the polypeptide linker comprises 5 amino acids.

16. A dimer of claim 15 wherein the polypeptide linker is Gly₄Ser.

17. An antibody multimer of claim 9 wherein the multimer is a trimer.

18. A trimer of claim 17 comprising three antigen binding fragments, wherein at least one of the antigen binding fragments is a Y1 scFv fragment or Y17 scFv fragment.

19. A trimer of claim 18 wherein the antigen binding fragments are linked by a polypeptide linker.

20. A trimer of claim 19 wherein the polypeptide linker comprises 1 to 5 amino acids.

21. An antibody multimer of claim 9 wherein the multimer is a tetramer.

22. A tetramer of claim 21 comprising four antigen binding fragments, wherein at least one of the antigen binding fragments is a Y1 scFv fragment or Y17 scFv fragment.

23. A tetramer of claim 22 wherein the antigen binding fragments are linked by a polypeptide linker.

24. A tetramer of claim 23 wherein the polypeptide linker comprises 1 to 5 amino acids.

25. A tetramer of claim 21 wherein the four antigen binding fragments form a complex through streptavidin-biotin association.

26. An antibody multimer of claim 9 comprising identical antigen binding fragments.

27. An antibody multimer of claim 9 wherein the at least first or second antigen binding fragment or both comprises a first hypervariable region comprising SEQ ID NO: 8.

28. An antibody multimer of claim 9 wherein the at least first or second antigen binding fragment or both comprises a first hypervariable region comprising SEQ ID NO:20.

29. An antibody multimer of claim 27 or 28 wherein the at least first or second antigen binding fragment or both has a second hypervariable region comprising SEQ ID NO: 115 and/or a third hypervariable region comprising SEQ ID NO: 114.

30. An antibody multimer of any one of claims 1, 4, 7, 27 and 28 wherein the multimer is capable of binding to at least two different molecules selected from the group consisting of PSGL-1, fibrinogen gamma prime (γ′), GP1bα, heparin, lumican, complement compound 4 (CC4), interalpha inhibitor, and prothrombin.

31. An antibody multimer of any one of claims 1, 4, 7, 27 and 28 wherein the multimer is capable of binding to at least two different molecules selected from the group consisting of PSGL-1, fibrinogen gamma prime (γ′), GP1bα, heparin, lumican, complement compound 4 (CC4), interalpha inhibitor, and prothrombin and is capable of binding to at least one cell type selected from the group consisting of B-CLL cells, AML cells, multiple myeloma cells, and metastatic cells.

32. An antibody dimer comprising a first and second antigen binding fragment, wherein said first or second antigen binding fragment or both comprises a hypervariable region comprising the amino acid sequence of SEQ ID NO: 8 [Y1 CDR3].

33. An antibody dimer comprising a first and second antigen binding fragment, wherein said first or second antigen binding fragment or both comprise a hypervariable region comprising the amino acid sequence of SEQ ID NO: 20 [Y17 CDR3].

34. An antibody dimer of claim 32 or 33, wherein said first or second antigen binding fragment or both further comprises a second hypervariable region comprising the amino acid sequence of SEQ ID NO:115 and/or a third hypervariable region comprising SEQ ID NO: 114.

35. An antibody multimer comprising a first and second antigen binding fragment, wherein said first or second antigen binding fragment or both is capable of cross-reacting with two or more epitopes, each epitope comprising one or more sulfated tyrosine residues and at least one cluster of two or more acidic amino acids.

36. An antibody multimer of claim 35 wherein said multimer is capable of cross-reacting with PSGL-1.

37. An antibody multimer of claim 35 that binds to QATEYEYLDYDFLPETE wherein at least one tyrosine residue is sulfated.

38. An antibody multimer of claim 35 wherein the multimer is capable of cross-reacting with GP1b-α.

39. An antibody multimer of claim 35 that binds to DEGDTDLYDYYPEEDTEGD wherein at least one tyrosine residue is sulfated.

40. An antibody multimer of claim 35 that binds to TDLYDYYPEEDTE wherein at least one tyrosine residue is sulfated.

41. An antibody multimer of claim 35 that binds to DEGDTDLYDYYP wherein at least one tyrosine residue is sulfated.

42. An antibody multimer of claim 35 that binds to to YDYYPEE wherein at least one tyrosine residue is sulfated.

43. An antibody multimer of claim 35 that binds to to TDLYDYYP wherein at least one tyrosine residue is sulfated.

44. An antibody multimer of claim 35 wherein the multimer is capable of cross-reacting with fibrinogen gamma prime.

45. An antibody multimer of claim 44 that binds to EPHAETEYDSLYPED wherein at least one tyrosine residue is sulfated.

46. An antibody multimer of claim 35 wherein the multimer is capable of cross-reacting with heparin.

47. An antibody multimer of claim 35 wherein the multimer is capable of cross-reacting with complement 4 (CC4).

48. An antibody multimer of claim 35 that is capable of cross-reacting with at least one cell selected from the group consisting of B-CLL cells, AML cells, multiple myeloma cells and metastatic cells.

49. A pharmaceutical composition comprising an antibody multimer according to any one of claims 1, 4, 7, 27 and 28.

50. A pharmaceutical composition of claim 49 comprising the antibody multimer in an effective amount to increase mortality of tumor cells or to increase the susceptibility of tumor cells to damage by an anti-cancer agent.

51. A pharmaceutical composition of claim 49 comprising the antibody multimer in an effective amount to inhibit growth and/or replication of leukemia cells.

52. A pharmaceutical composition of claim 49 comprising the antibody multimer in an effective amount to inhibit abnormal cell-cell, cell-matrix, platelet-matrix, platelet-platelet, and/or platelet-cell adhesion.

53. A pharmaceutical composition of claim 49 comprising the antibody multimer in an effective amount to increase the susceptibility of diseased cells to damage by anti-disease agents.

54. A pharmaceutical composition of claim 49 comprising the antibody multimer in an effective amount to increase the mortality of leukemia cells amount or to increase the susceptibility of leukemia cells to damage by anti-leukemia agents.

55. A pharmaceutical composition comprising an antibody multimer according to any one claims 1, 4, 7, 27 and 28 coupled to or complexed with an agent selected from the group consisting of anti-cancer, anti-metastasis, anti-leukemia, anti-disease, anti-adhesion, anti-thrombosis, anti-restenosis, anti-auto-immune, anti-aggregation, anti-bacterial, anti-viral, and anti-inflammatory agents.

56. A pharmaceutical composition of claim 55 wherein the agent is selected from the group consisting of toxins, radioisotopes and pharmaceutical agents.

57. A pharmaceutical composition of claim 55 wherein the agent is an anti-viral agent selected from the group consisting of acyclovir, ganciclovir and zidovudine.

58. A pharmaceutical composition of claim 55 wherein the agent is an anti-thrombosis/anti-restenosis agent selected from the group consisting of cilostazol, dalteparin sodium, reviparin sodium, and aspirin.

59. A pharmaceutical composition of claim 55 wherein the agent is an anti-inflammatory agent selected from the group consisting of zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid.

60. A pharmaceutical composition of claim 55 wherein the agent is an anti-autoimmune agent selected from the group consisting of leflunomide, denileukin diftitox, subreum, WinRho SDF, defibrotide, and cyclophosphamide.

61. A pharmaceutical composition of claim 55 wherein the agent is an anti-adhesion/anti-aggregation agent selected from the group consisiting of limaprost, clorcromene, and hyaluronic acid.

62. A pharmaceutical composition of claim 56 wherein the the radioisotope is selected from the group consisting of gamma-emitters, positron-emitters, x-ray emitters, beta-emitters, and alpha-emitters.

63. A pharmaceutical composition of claim 62 wherein the wherein the radioisotope is selected from the group consisting of ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}telluriunm ¹⁶⁵thulium, ¹⁶⁷thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium, ¹⁰⁷mercury, ²⁰³mercury, ⁶⁷gallium and ⁶⁸gallium.

64. A pharmaceutical composition of claim 56 wherein the pharmaceutical agent is selected from the group consisting of doxorubicin, methoxymorpholinyldoxorubicin (morpholinodoxorubicin), adriamycin, cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide and bleomycin, and derivatives and combinations thereof.

65. A pharmaceutical agent of claim 55 coupled to or complexed with a vehicle or carrier that is capable of being coupled or complexed to more than one agent.

66. A pharmaceutical composition of claim 65 wherein the vehicle or carrier is selected from the group consisting of dextan, lipophiolic polymers, HPMA and liposomes.

67. A pharmaceutical composition of claim 49 comprising the antibody multimer in an amount effective to inhibit cell rolling.

68. A pharmaceutical composition of claim 48 comprising the antibody multimer in an amount effective to inhibit inflammation.

69. A pharmaceutical composition of claim 48 comprising the antibody multimer in an amount effective to inhibit auto-immune disease.

70. A pharmaceutical composition of claim 48 comprising the antibody multimer in an amount effective to inhibit thrombosis.

71. A pharmaceutical composition of claim 48 comprising the antibody multimer in an amount effective to inhibit restenosis.

72. A pharmaceutical composition of claim 48 in an amount effective to inhibit metastasis.

73. A pharmaceutical composition of claim 48 comprising the antibody multimer in an amount effective to inhibit growth and/or replication of tumor cells, increase mortality of tumor cells, or increase the susceptibility of tumor cells to damage by anti-cancer agents.

74. A pharmaceutical composition of claim 49 comprising the antibody multimer in an amount effective to inhibit growth and/or replication of leukemia cells, increase the mortality rate of leukemia cells or increase the susceptibility of leukemia cells to damage by anti-leukemia agents.

75. A pharmaceutical composition of claim 49 comprising the antibody multimer in an amount effective to increase the susceptibility of diseased cells to damage by anti-disease agents.

76. A pharmaceutical composition of claim 49 comprising the antibody multimer in an amount effective to inhibit cell-cell, cell-matrix, platelet-matrix, platelet-platelet, and/or cell-platelet aggregation, adhesion or complex formation.

77. A pharmaceutical composition of claim 49 coupled to or complexed with an agent selected from the group consisting of anti-cancer, anti-metastasis, anti-leukemia, anti-disease, anti-adhesion, anti-thrombosis, anti-restenosis, anti-autoimmune, anti-aggregation, anti-bacterial, anti-viral, and anti-inflammatory agents.

78. A pharmaceutical composition of claim 78 wherein the agent is an anti-viral agent selected from the group consisting of acyclovir, ganciclovir and zidovudine.

79. A pharmaceutical composition of claim 49 wherein the antibody multimer is coupled to or complexed with a vehicle or carrier that is capable of being coupled or complexed to more than one agent.

80. A pharmaceutical composition of claim 49 wherein the vehicle or carrier is selected from the group consisting of dextran, lipophilic polymers, HPMA, and liposomes.

81. A method of inhibiting cell rolling, comprising administering to a patient in need thereof a pharmaceutical composition comprising an effective amount of an antibody multimer according to any one of claims 1, 4, 7, 27 and 28.

82. A method of inhibiting inflammation, comprising administering to a patient in need thereof a pharmaceutical composition comprising an effective amount of an antibody multimer according to any one of claims 1, 4, 7, 27 and 28.

83. A method of inhibiting auto-immune disease, comprising administering to a patient in need thereof a pharmaceutical composition comprising an effective amount of an antibody multimer according to any one of claims 1, 4, 7, 27 and 28.

84. A method of inhibiting metastasis, comprising administering to a patient in need thereof a pharmaceutical composition comprising an effective amount of an antibody multimer according to any one of claims 1, 4, 7, 27 and 28.

85. A method of inhibiting thrombosis, comprising administering to a patient in need thereof a pharmaceutical composition comprising an effective amount of an antibody multimer according to any one of claims 1, 4, 7, 27 and 28.

86. A method of inhibiting restenosis, comprising administering to a patient in need thereof a pharmaceutical composition comprising an effective amount of an antibody multimer according to any one of claims 1, 4, 7, 27 and 28.

87. A method of inhibiting growth and/or replication of tumor cells or increasing the susceptibility of tumor cells to damage by anti-cancer agents, comprising administering to a patient in need thereof a pharmaceutical composition comprising an effective amount of an antibody multimer according to any one of claims 1, 4, 7, 27 and 28.

88. A method of inhibiting growth and/or replication of leukemia cells or increasing the susceptibility of leukemia cells to damage by anti-leukemia agents, comprising administering to a patient in need thereof a pharmaceutical composition comprising an effective amount of an antibody multimer according to any one of claims 1, 4, 7, 27 and 28.

89. A method of increasing the susceptibility of diseased cells to damage by anti-disease agents, comprising administering to a patient in need thereof a pharmaceutical composition comprising an effective amount of an antibody multimer according to any one of claims 1, 4, 7, 27 and 28.

90. A method of inhibiting cell-cell, cell-matrix, platelet-matrix, platelet-platelet, and/or cell-platelet complex formation, aggregation, or adhesion comprising administering to a patient in need thereof a pharmaceutical composition comprising an effective amount of an antibody multimer according to any one of claims 1, 4, 7, 27 and 28.

91. A method of ameliorating the effects of disease, preventing disease, treating a disease or inhibiting the progress of a disease, comprising administering to a patient in need thereof a pharmaceutical composition comprising an effective amount of an antibody multimer according to any one of claims 1, 4, 7, 27 and 28.

92. A method of claim 91 wherein the antibody multimer is coupled to or complexed with an agent selected from the group consisting of anti-cancer, anti-metastasis, anti-leukemia, anti-disease, anti-adhesion, anti-thrombosis, anti-restenosis, anti-autoimmune, anti-aggregation, anti-bacterial, anti-viral, and anti-inflammatory agents.

93. A method of claim 92 wherein the agent is an anti-viral agent selected from the group consisting of acyclovir, ganciclovir and zidovudine; an anti-thrombosis/anti-restenosis agent selected from the group consisting of cilostazol, dalteparin sodium, reviparin sodium, and aspirin; an anti-inflammatory agent selected from the group consisting of zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; or an anti-autoimmune agent selected from the group consisting of leflunomide, denileukin diftitox, subreum, WinRho SDF, defibrotide, and cyclophosphamide; an anti-adhesion/anti-aggregation agent selected from the group consisiting of limaprost, clorcromene, and hyaluronic acid.

94. A method of claim 92, wherein the agent is selected from the group consisting of toxins, radioisotopes, and pharmaceutical agents.

95. A method of claim 94, wherein the pharmaceutical agent is selected from the group consisting of doxorubicin, methoxymorpholinyldoxorubicin (morpholinodoxorubicin), adriamycin, cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide and bleomycin, and derivatives and combinations thereof.

96. A method according to claim 92, wherein the antibody multimer is coupled to or complexed with a vehicle or carrier that is capable of being coupled or complexed to more than one agent.

97. A method according to claim 96, wherein the vehicle or carrier is selected from the group consisting of dextran, lipophilic polymers, HPMA, and liposomes.

98. A kit comprising at least one antibody multimar according to any one of claim 1, 4, 7, 27 and 28.
